(19) United States
(12) Patent Application Publication
(10) Pub. No.: US 2022/0273823 A1
(43) Pub. Date: Sep. 1, 2022

(54) THERANOSTIC CONJUGATES

(71) Applicant: Ariel Scientific Innovations Ltd., Ariel (IL)

(72) Inventors: Gary GELLERMAN, Rishon-LeZion (IL); Leonid D. PATSENKER, Ariel (IL); Andrii BAZYLEVICH, Ariel (IL); Aleksey ROZOVSKY, Rehovot (IL)

(73) Assignee: Ariel Scientific Innovations Ltd., Ariel (IL)

(21) Appl. No.: 17/627,673

(22) PCT Filed: Jul. 15, 2020
(86) PCT No.: PCT/IL2020/050795

§ 371 Date: Jan. 16, 2022

Related U.S. Application Data
(60) Provisional application No. 62/874,580, filed on Jul. 16, 2019.

(51) Int. Cl. A61K 49/00; A61K 47/55

(52) U.S. Cl. CPC A61K 49/0058; A61K 49/0041; A61K 49/0036; A61K 49/0032; A61K 49/0021; A61K 49/0056; A61K 47/552; A61K 49/003

(57) ABSTRACT

Provided herein is a drug delivery (DD) system for ratiometric luminescence determination of drug release degree in drug delivery monitoring, which includes a drug, a switchable reporter and non-switchable reporter providing two distinguishable signals for detection; or a single switchable reporter providing two distinguishable signals for detection, and a cleavable linker connecting a drug to a switchable reporter, as well as a method for ratiometric luminescence determination of drug release in a target (in vivo or in vitro), which is effected by administering the DD system provided herein that is capable of releasing a drug from the DD system, measuring two luminescent signals provided by the switchable reporter and the non-switchable reporter, or the single switchable reporter, determining the ratio between these two luminescence signals, and determining the drug release degree through the ratio between the two luminescence signals.

### RELATED APPLICATIONS

This application claims the benefit of priority of U.S. Provisional Patent Application No. 62/874,580 filed on 16 Jul. 2019, the contents of which are incorporated herein by reference in their entirety.

### FIELD AND BACKGROUND OF THE INVENTION

The present invention, in some embodiments thereof, relates to a class of theranostic conjugates, and more particularly, but not exclusively, to compounds capable of targeted drug delivery of a bioactive agent while providing quantitative drug delivery signals.

Targeted delivery of anti-cancer drugs, as compared to conventional chemotherapy, improves therapeutic efficacy and minimize side effects caused by nonspecific drug distribution. A basic targeted drug delivery (TDD) system comprises an anticancer drug covalently bound to a cancer-specific carrier such as peptide, antibody or nanoparticle, for target cell recognition and addressed drug delivery to tumor tissue. Combining this classical TDD system with a fluorescent reporter provides the real-time monitoring of drug transport, which can be used in theranostics (combination of therapy and diagnostics) and personalized medicine. Employing a turn-on fluorogenic reporter bound to the drug by means of a biodegradable linker such as an ester group, carbamate or carbonate, enables estimation of the efficacy and accuracy of drug release in target cells. The environment driven cleavage of the biodegradable linker, which is initiated, e.g. by esterase, causes a noticeable increase in the fluorescence intensity of the fluorogenic reporter, signaling the drug release event. Thereby, the fluorescence signal generated by the dye can be utilized for monitoring of drug transport and drug release.

Research efforts are dedicated to preparing TDD conjugates composed of switchable fluorescent reporter bound to a drug by means of a biodegradable cleavable linker, and to a targeting carrier by means of a non-cleavable linker. However, switchable fluorophores used for TDD systems such as coumarin, BODIPY, 1,8-naphthalimide, fluorescein, etc. suffer from short wavelength emission, which is not suitable for monitoring the drug release in vivo.

As a switchable NIR reporter, xanthene-cyanine dyes were recently designed for spectrofluorometric and imaging based detection of peroxynitrite, fibroblasts, aminopeptidase, nitroxyl, hydrogen polysulfide, carboxylesterase, and glutathionine. These reporters were proposed also for chemotherapy and keloid diagnosis; however, these dyes do not contain reactive functionalities for binding to a targeting carrier and until now none of them were used for TDD monitoring.

Relevant documents drawn to such reporters include, Zhang, J. et al., Anal. Chem. 2018, 90, 9301-9307; Miao, Q. et al., Angew. Chemie Int. Ed. 2018, 57, 1256-1260; He, X. et al., Chem. Sci. 2017, 8, 3479-3483; He, X. et al., Chem. Commun. 2017, 53, 9438-9441; Tan, Y. et al., Sci. Rep. 2015, 5, 1-9; Fang, Y. et al., Chem. Commun. 2017, 53, 8759-8762; Li, D. et al., J. Agric. Food Chem. 2017, 65, 4209-4215; Xie, J.-Y et al., Anal. Chem. 2016, 88, 9746-9752;

Kong, F. et al., Anal. Chem. 2016, 88, 6450-6456; Liu, X. et al., Chem. Sci. 2017, 8, 7689-7695; and Cheng, P. et al., Chem. Sci. 2018, 9, 6340-6347.

Currently available theranostic platforms suffer from two major caveats. First, their ability to quantitatively monitor TDD is limited, due to (a) their inability to measure the ratio between the released drug molecules (chemotherapeutic drug or PDT sensitizer) and the total number of theranostic conjugate molecules accumulating in tissue sites (see, background art in FIG. 1); and (b) the poor dynamic range of the change in the fluorescence intensity or sensitizing efficacy upon drug release. Second, currently used reporters are not sufficiently bright within the red-NIR spectral region, which decreases the signal-to-noise ratio.

Quantitative measurements, i.e. determination of the drug release ratio or drug release degree (the ratio between the number of drug molecules released in the target cells and the number of TDD conjugates accumulated in tissue sites), is problematic. This is due to the fact that the fluorescence intensity of the dye is dependent not only on the total concentration of the TDD conjugate molecules delivered to target tissue, but it is also affected by the light absorption and scattering in the body and therefore is a function of the light path depth. Luminescence lifetime of a dye is not dependent of the dye concentration and therefore cannot be utilized as a parameter for determination of drug release degree. The ratiometric measurements utilizing two fluorescence signals measured at the two different wavelengths or two fluorescence lifetimes are known to improve quantitation in biological matter. The ratiometric measurements provide effective internal referencing and self-calibration that greatly improve sensitivity, reliability and quantification in biological samples. Ratiometry is unaffected by the sample nature and the instrumentation. [e.g., Yang, Y. et al., ACS Sensors, 2018, 3, 2278-2285; Lee M. H., et al. Chem. Soc. Rev., 2015, 44, 4185-4191; Wang X. et al., Chem. Sci., 2013, 4, 2551-2556; Yu, F. et al., Chen, Biomaterials, 2015, 63, 93-101].

Guo, Z. et al. [Chem. Sci., 2012, 3, 2760-2765] report a highly selective ratiometric near-infrared fluorescent cyanine sensor for cysteine with remarkable shift and its application in bioimaging.

### SUMMARY OF THE INVENTION

The present invention provides, inter alia, a solution to the problems stemming from the inability to measure the ratio between the released drug molecules (chemotherapeutic drug or PDT sensitizer) and the total number of theranostic conjugate molecules accumulating in tissue sites, and problems associated with the poor dynamic range of the change in the fluorescence intensity or sensitizing efficacy upon drug release, by providing a quantitative ratiometric fluorescence monitoring of TDD to improve the precision and efficacy of chemical and photodynamic anti-cancer therapy; providing prototypes of the dual-dye theranostic platforms, and the data obtained on the synthesis and evaluation of these conjugates, can be used to develop efficient tools for treating a wide variety of cancer, microbial, and viral deceases; the ability to utilize a wide variety of drugs, reporters, sensitizers, targeting peptides, and antibodies; providing highly bright switchable reporters with an increased fluorescence intensity dynamic range that can be used both in conventional theranostic platforms and in the novel, dual-dye platform; and also providing activatable sensitizers that can improve the safety (i.e., reduce side-effects) and precision of PDT.

Aspects so the present invention are drawn to a family of compounds that share the common property of allowing a quantitative and self-calibrated monitoring of a targeted drug in vitro and in vivo, using fluorescent light.

Thus, according to an aspect of some embodiments of the present invention there is provided conjugate that includes:

a bioactive agent moiety,

at least one fluorophore moiety, and

a cleavable linker connecting the bioactive agent moiety and the at least one fluorophore moiety, wherein:

the at least one fluorophore moiety is characterized by at least one reference luminescence signal and at least one switchable luminescence signal, and a change in the switchable luminescence signal upon cleavage of the cleavable linker is different than a change in the reference luminescence signal,

the conjugate is structured and designed so as to allow monitoring and calibrated luminescence determination of a value related to a release of the bioactive agent from the conjugate.

According to some embodiments of the invention, the conjugate includes at least two fluorophore moieties, wherein at least one of the at least two fluorophore moieties is characterized by exhibiting the reference luminescence signal (a reference fluorophore moiety), and at least one other of the at least two fluorophore moieties is characterized by exhibiting the switchable luminescence signal (a switchable fluorophore moiety).

According to some embodiments of the invention, the conjugate includes a single fluorophore moiety that is characterized by exhibiting the reference luminescence signal and the switchable luminescence signal.

According to some embodiments of the invention, each of the luminescence signals is independently detectable within a range from 600 nm to 900 nm.

According to some embodiments of the invention, each of the reference luminescent signal and the switchable luminescence signal that includes at least one distinguishable luminescence intensity of at least one wavelengths, and/or at least one distinguishable luminescence lifetime, and/or at least one distinguishable polarization/anisotropy, and any combination, ratio, product and/or correlation thereof.

According to some embodiments of the invention, the change in the switchable luminescence signal is at least 10% greater than the change in the reference luminescence signal.

According to some embodiments of the invention, the conjugate presented herein is structured and designed so as to allow theranostic bioavailability at physiological conditions.

According to some embodiments of the invention, the conjugate presented herein further includes a targeting moiety.

According to some embodiments of the invention, the fluorophore moiety that exhibits at least two luminescence signals, both reference and switchable, is selected from the group consisting of:

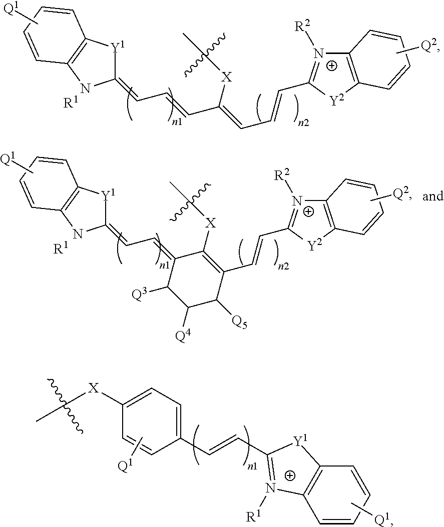

wherein:

X═O, S, Se, NR^{N}, 2-phenyoxy, 4-phenyoxy, aryloxy;

R^{N}=hydrogen, alkyl, aryl alkylaryl, or contain a reactive group or a solubilizing group selected from sulfate, sulphonate, quaternary amine, phosphate, phosphonate and PEG;

Y¹, Y² are independently selected from C(R^{a}, R^{b}), O, S, NR^{N};

R^{a}, R^{b} are independently selected from hydrogen, alkyl, aryl alkylaryl, a contain a reactive group or solubilizing group selected from sulfate, sulphonate, quaternary amine, phosphate, phosphonate and PEG;

R^{a} and R^{b} can form a ring;

R¹, R² are independently selected from hydrogen, alkyl, aryl alkylaryl, or contain a reactive group or a solubilizing group selected from sulfate, sulphonate, quaternary amine, phosphate, phosphonate and PEG;

Q¹, Q² are at least one of groups consisting of R¹, halogen, cyano, sulfo, phosphate, carboxy, formyl, alkyl, aryl, alkylaryl, alkoxy, aryloxy or a substituted or unsubstituted cyclic moiety; two adjacent Q¹ and two adjacent Q² can form a substituted or unsubstituted cyclic moiety;

each of

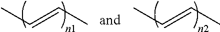

is independently a linear or cyclic, substituted or unsubstituted polyene, and each of n1 and n2 is independently an integer ranging 1-4; and

the wiggled line represents attachment to the cleavable linker.

According to some embodiments of the invention, the switchable fluorophore moiety is selected from the group consisting of:

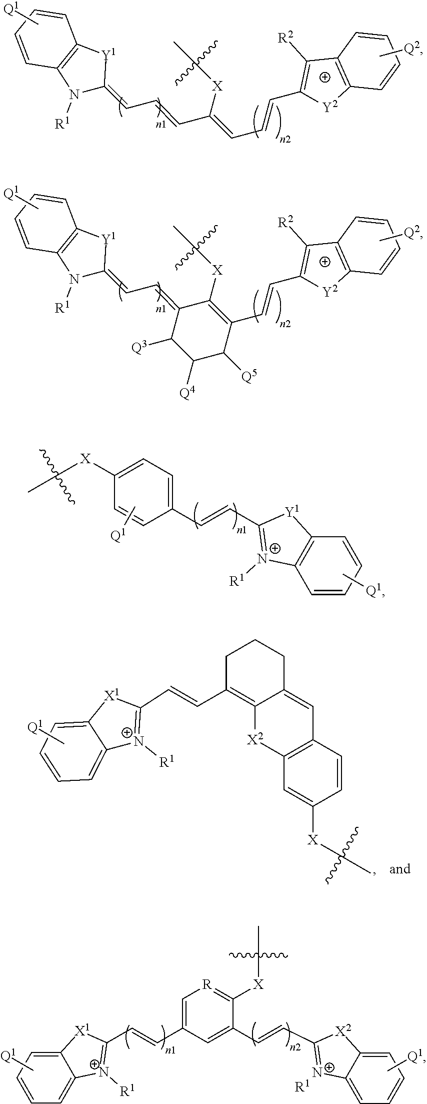

wherein:

X═O, S, Se, NR^{N}, 2-phenyoxy, 4-phenyoxy, aryloxy;

R^{N}=hydrogen, alkyl, aryl alkylaryl, or contain a reactive group or a solubilizing group selected from sulfate, sulphonate, quaternary amine, phosphate, phosphonate and PEG;

Y¹, Y² are independently selected from C(R^{a}, R^{b}), O, S, NR^{N};

R^{a}, R^{b} are independently selected from hydrogen, alkyl, aryl alkylaryl, or contain a reactive group or a solubilizing group selected from sulfate, sulphonate, quaternary amine, phosphate, phosphonate, PEG;

R^{a} and R^{b} can form a ring;

R¹, R² are each independently selected from hydrogen, alkyl, aryl alkylaryl, or contain a reactive group or a solubilizing group selected from sulfate, sulphonate, quaternary amine, phosphate, phosphonate, PEG;

Q¹, Q² are at least one of groups consisting of R¹, halogen, cyano, sulfo, phosphate, carboxy, formyl, alkyl, aryl, alkylaryl, alkoxy, aryloxy or a substituted or unsubstituted cyclic moiety; two adjacent Q¹ and two adjacent Q² can form a substituted or unsubstituted cyclic moiety;

each of

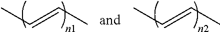

is independently a linear or cyclic, substituted or unsubstituted polyene, and each of n1 and n2 is independently an integer ranging 1-4; the wiggled line represents attachment to the cleavable linker.

According to some embodiments of the invention, the reference fluorophore moiety that includes a fluorescent dye selected from the group consisting of a cyanine-based fluorescent dye, a styryl-based fluorescent dye, a squaraine-based fluorescent dye, a squaraine-rotaxane-based fluorescent dye, a phthalocyanine-based fluorescent dye, a porphyrine-based fluorescent dye, a xanthene-based dye, a phenothiazine-based dye, a luminescent metal-ligand complex, a fluorescent protein, a luminescent nanoparticle, a luminescent quantum dot, a luminescent nanocrystal, a luminescent polymeric particle, a tandem fluorophore, or a fluorescent dye selected from Cy, Dy, Alexa Fluor, IRDye, LiCor, BODIPY, SETA dye series.

According to some embodiments of the invention, the targeting moiety is selected from the group consisting of a peptide, a protein, an antibody and a nanoparticle.

According to some embodiments of the invention, the targeting moiety is selected from the group consisting of octreotide (OCT), lanreotide, pasireotide, vapreotide, cilengitide analog c(RGDfK), and luteinizing Hormone-Releasing Hormone (LHRH), bombesin, and arginine-glycine-aspartic acid (RGD).

According to some embodiments of the invention, the conjugate presented herein further includes a spacer moiety linking the targeting moiety and the at least one fluorophore moiety.

According to some embodiments of the invention, the cleavable linker includes an ester, an amide, a carbamate, a carbonate, a disulfide, a sulfonamide, an ether, a thioether, a valine-citrulline, a hydrazine and an oxyacrylate.

According to some embodiments of the invention, the bioactive agent is selected from the group consisting of a drug, a photodynamic therapy sensitizer, radiotherapy agent, a metal complex, an anti-cancer agent, an anti-proliferative agents, chemosensitizing agents, an anti-inflammatory agent, an antimicrobial agent, an anti-oxidant, a hormone, an anti-hypertensive agent, an anti-diabetic agent, an immunosuppressant, an enzyme inhibitor, a neurotoxin and an opioid.

According to some embodiments of the invention, the bioactive agent is a drug selected from the group consisting of chlorambucil, azatoxin, an antimitotic, dolastatin 10, monomethyl auristatin F, monomethyl auristatin E, maytansine (DM1), a Topo I irinotecan inhibitor, 7-ethyl-10-hydroxy-camptothecin (SN-38), a DNA minor groove binding alkylating agent, duocarmycin, adozelesin, bizelesin and carzelesin.

According to some embodiments of the invention, the sensitizer is photo-activated upon cleavage of the cleavable linker.

According to some embodiments of the invention, the sensitizer includes a dye selected from the group consisting of a cyanine-based dye, a styryl-based dye, a squaraine-based dye, a phthalocyanine-based dye, a porphyrine-based dye, xanthene-based dye, a phenothiazine-based dye, a iodinated dye, a brominated dye, a chlorine-based dye, a bacteriochlorin-based dye, a fullerene-based dye, a metal-ligand complex, a halogenated dye, a nanoparticle, a photofrin-based dye, a photoporphyrin-based dye, a benzoporphyrin-based dye, a tookad-based dye, an antrin-based dye, a purlytin-based dye, a foscan-based dye, a iodinated, brominated or a mixed iodinated-bromitated (e.g., containing both Br and I) cyanine-based or squaraine based dye, and any combination thereof.

According to another aspect of some embodiments of the present invention there is provided a method of calibrated luminescence determination of a value related to a release of a bioactive agent in a tissue, the method is effected by:

scanning the tissue with a probe designed to detect and record the reference luminescence signal and the switchable luminescence signal;

contacting the tissue with the conjugate provided herein;

monitoring a change in the reference luminescence signal and the switchable luminescence signal for a predetermined period of time;

calculating the value related to a release of the bioactive agent according to the following equation:

R_{eff}˜I_{Swi signal}/I_{Ref signal}, or

R_{eff}=k(I_{Swi signal}/I_{Ref signal})

wherein:

I_{Swi signal} is a value representing switchable luminescence signal, which may comprise intensity values of the switchable luminescence signal or contribution factor to the fluorescence lifetime of the switchable luminescence signal (τ_{Swi signal}), or any mathematical combination, correlation or product thereof,

I_{Ref signal} is a value representing reference luminescence signal, which may comprise intensity of the reference luminescence signal or contribution factor to the fluorescence lifetime of the reference luminescence signal (τ_{Ref signal}), or any mathematical combination, correlation or product thereof,

τ_{Mean signal}=(I_{Swi signal}×τ_{Swi signal})+(I_{Ref signal}×τ_{Swi signal}), where τ_{Mean signal} is a mean luminescence lifetime,

and

k is an experimentally determined calibration coefficient, which takes into account different brightness of the switchable and reference fluorophores and different light absorption at two different wavelengths among other factors.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents simplified illustrations of some background art, showing theranostic conjugates and drug release in target cells, wherein “reporter” is a switchable fluorescent dye (background art);

FIG. 2A-D present simplified illustrations of some embodiments of the present invention, wherein FIG. 2A presents simplified illustrations of five exemplary and non-limiting embodiments of the present invention, wherein each of the embodiments exhibits the elements of a reference fluorophore, a bioactive agent, switchable fluorophore and a cleavable linker linking the two, whereas in some embodiments, some of the elements take more than one role, and act as both a fluorophore and a bioactive agent, FIG. 2B shows the principle of functioning of a quantitative ratiometric fluorescence monitoring of targeted drug delivery (TDD) conjugate, according to some embodiments of the present invention, comprising a bioactive agent in the form of the anti-cancer drug CLB or an activatable PDT sensitizer, a targeting moiety in the form of the peptide OctA, and a single fluorophore in the form of the switchable dual-fluorescent dye IRD that enables ratiometric measurements, FIG. 2C presents an exemplary dual-dye (two fluorophores) theranostic conjugate for TDD of a chemotherapeutic drug or an activatable PDT sensitizer, according to some embodiments of the present invention, and FIG. 2D presents an exemplary dual-dye theranostic conjugate of an activatable sensitizer;

FIG. 3 presents a simplified illustration of the quantification of R_{eff} using a dual-dye theranostic conjugates and approach, according to some embodiments of the present invention, wherein the “Switchable” and “Swi-on” refer to the switchable fluorophore moiety, and the “Reference” and “Ref” refer to the reporter fluorophore moiety;

FIG. 4 presents a plot showing the spectral properties of “turn-on” switchable dye XCy in PB (line A), XCy in CM (line B), XCy-CLB in PB (line C), XCy-CLB in CM (line D), OCTA-G-XCy-CLB in PB (line E), and OCTA-G-XCy-CLB in CM (line F), and (line A) and (line B)—XCy exists in the “on” form; (line C), (line D) and (line E)—XCy exists in the “off” form;

FIGS. 5A-H present time dependent absorption (FIGS. 5A, C, E and G) and fluorescence (FIGS. 5B, D, F and H) spectra of XCy-CLB (FIGS. 5A, B, C and D) and OCTA-G-XCy-CLB (FIGS. 5E, F, G and H) in PB (FIGS. 5A, B, E and F) and CM (FIGS. 5C, D, G and H), whereas λₑₓ=650 nm;

FIGS. 6A-B present spectrophotometrically (Abs) and spectrofluorometrically (Fl) estimated cleavage profiles for conjugate XCy-CLB and OCTA-G-XCy-CLB in PB pH 7.4 (FIG. 6A) and cell medium (FIG. 6B);

FIGS. 7A-C present drug (CLB) release profiles measured by relative fluorescence intensities (RFI) of selected Panc-1 and CHO cells (FIG. 7A), showing that the CLB cleavage half-life is τ_{1/2}˜25 min for Panc-1 and τ_{1/2} ˜2.5 h for CHO, and the cell inhibition of Panc-1 (FIG. 7B) and CHO (FIG. 7C) pre-treated with various concentrations of OCTA-G-XCy-CLB, free CLB and Free OCTA, whereas after the treatment, the cells were incubated for 24 h and 48 h at 37° C., cell inhibition was accessed using standard XTT assay, and the inhibition for each concentration point is represented by the mean±standard error for each independent experiment conducted in triplicate;

FIG. 8 presents a normalized absorption and emission spectra of RD, IRD-CLB, and 5-CLB measured at c=0.6 μM in PB (solid line) and CM (dashed line), whereas the excitation wavelength was 532 nm for RD and 720 nm for IRD-CLB and 5;

FIGS. 9A-D present a plot showing the time-dependent fluorescence spectra at T=25° C. of IRD-CLB (FIGS. 9A, B) and 5-CLB (FIGS. 9C, D) in PB (FIGS. 9A, C) and CM (FIGS. 9B, D);

FIGS. 10A-B present comparative plots showing the CLB cleavage profiles (FIG. 9A) and ratiometric curves (F_{Red}/F_{NIR}) (FIG. 9B) for IRD-CLB and 5-CLB (c=0.6 μM) measured in PB (solid line) and CM (dashed line) at 25° C. after incubation at 37° C.;

FIG. 11 presents a comparative plot, showing inhibition of the PANC-1 growth by 5-CLB, CLB and OctA, wherein at the end of 20 minutes incubation period and subsequent washing, cell growth was assessed using the XTT assay at 24 hours (the inhibition for each concentration point is represented by the mean±standard error for each independent experiment conducted in triplicate);

FIG. 12 presents a comparative plot showing a decrease of the normalized fluorescence intensity of PANC-1 after incubation with 5-CLB (10 μM) and Oct at different [Oct]/[5-CLB] ratios at 60 min after incubation, wherein the fluorescence intensity for each concentration point was measured in the NIR channel and represented by the mean±standard error for three independent experiments;

FIG. 13 presents a comparative plot showing the CLB cleavage profiles obtained by fluorescence imaging of PANC-1 cell line stained with 5-CLB, wherein the plot marked by “1” shows the decrease of the brightness B_{NIR}, the plot marked as “2” shows an increase of the brightness B_{Red}, the plot marked as “3” shows ratiometric curve (B_{Red}/B_{NIR}), and, the plot marked as “4” shows ratiometric curve in logarithmic scale [lg(B_{Red}/B_{NIR})];

FIGS. 14A-B present absorption (dashed line) and fluorescence (solid line) spectra of representative reference reporter and switchable dye (FIG. 14A), and the anticipated experimental fluorescence spectra affected by FRET (FIG. 14B); and

FIG. 15. presents the fluorescence emission profiles for drug release from dual-dye conjugate Aza-FLU-Cy5 in cell culture medium, wherein the dashed line represents the fluorescein (FLU) emission intensity, I_{FLU} (excitation 485 nm), the dotted line represents Cy5 emission intensity, I_{Cy5} (excitation 610 nm) and the solid line represents R_{eff.} ˜I_{FLU}/I_{Cy5}.

### DESCRIPTION OF SOME SPECIFIC EMBODIMENTS OF THE INVENTION

The present invention, in some embodiments thereof, relates to a class of theranostic conjugates, and more particularly, but not exclusively, to compounds capable of targeted drug delivery of a bioactive agent while providing quantitative drug delivery signals.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As presented hereinabove, theranostics, a combination of therapy and diagnostics, facilitates personalized medicine and allows disease-targeting treatments, including for cancer patients. For these patients, theranostics comprises diagnostic procedures that recognize the abnormal, cancerous cells, followed by targeted drug delivery (TDD), monitoring of the drug release, and treatment. A basic theranostic platform comprises a drug, a targeting group for recognizing the abnormal cells, and a diagnostic reporter (imaging agent); these three elements are bound to each other by linkers and, together, form the theranostic conjugate. The drug can be a cytotoxic chemical compound (chemotherapy) or it can refer to oxidation by light-generated cytotoxic species (i.e., singlet oxygen or free radicals, which can kill cancer cells and other pathogens, facilitated by organic dyes (sensitizes) under light exposure (photochemotherapy or photodynamic therapy, PDT). The targeting group is usually an antibody (Ab), a peptide, or a nanoparticle, which can bind specifically to overexpressed receptors that are abundant in cancer cells. The diagnostic reporter enables in vitro or in vivo monitoring of the theranostic conjugate by various diagnostic imaging methods.

One of the most sensitive, non-toxic, non-harmful, and inexpensive noninvasive methods of in vivo diagnostics is fluorescence imaging, which can detect analytes in the nano- and picomolar range. Fluorescence imaging requires a fluorescent reporter: a dye molecule that absorbs and emits light as a signal for detection, as used in many biomedical assays and applications. The dye molecule consists of a fluorophore moiety, which is responsible for light absorption, fluorescence, and the generation of cytotoxic species, and substituents that provide covalent binding and hydrophobic-hydrophilic properties. Several key features of fluorescent dyes (reporters and sensitizers) are required for theranostic applications, and, particularly, in the context of the present invention:

Spectral range: The dye must absorb and emit light in the near-infrared (NIR) spectral region (˜650-850 nm), where biological matter has minimal auto-absorption and auto-fluorescence. Excitation and luminescence within this spectral range improve the signal-to-noise ratio and minimize tissue damage. Therefore, in the context of some embodiments of the present invention, the inventors have utilized NIR dyes.

Brightness: Upon excitation, the dye used as the reporter must be sufficiently bright to reliably detect small amounts of analytes through skin and tissue. The brightness (B) of the reporter can be quantified as its extinction coefficient, ε (i.e., the efficacy to absorb light) multiplied by the luminescence quantum yield, Φ_{F} (i.e., the efficacy to emit light): B=ε×Φ_{F}. The brightness of the reporter is a crucial parameter in luminescence-based theranostics and, together with the spectral range, it is responsible for the signal-to-noise ratio and for the sensitivity of the method to low analyte concentration. For biomedical assays and diagnostics, brightness is suggested to be at least 50,000 M⁻¹ cm⁻¹, which is achievable in the NIR range with cyanine dyes. Therefore, in the context of some embodiments of the present invention, the inventors have utilized cyanine.

Photostability: The dye must be photostable, so as to avoid photobleaching and photodecomposition during diagnosis and treatment.

Biostability: The dye must not be affected by oxidation, reduction, or other metabolic reactions.

Chemostability: The dye must be stable in the conjugation reactions.

Phototoxicity: Upon excitation, some dyes may generate reactive cytotoxic species (e.g., singlet oxygen and organic or inorganic free radicals), which can damage cells and tissues. Therefore, the reporter must be non-phototoxic. Notably, however, some highly phototoxic dyes can be used as sensitizers in PDT for both therapy and diagnostic applications, as proposed herein.

Cytotoxicity: The reporter must not be cytotoxic and the sensitizer must not be cytotoxic in the dark.

Solubility: As the aggregation of dye molecules decreases their brightness and efficacy to generate singlet oxygen and free radicals, both the reporter and sensitizer must be sufficiently soluble in biological media and in the target cells. The solubility in aqueous media can be increased by introducing hydrophilic, solubilizing groups (such as sulfonic, phosphonic, ammonium, or polyether (PEG) groups) to the dye. In addition, both the lipid and water solubility of the dyes and dye-conjugates should be adjusted to increase their permeability through cell membranes, decrease background interference by lowering noncovalent hydrophobic binding to proteins in the blood, and reduce toxicity to normal cells.

Reactivity: To assemble a theranostic conjugate, the dye must contain reactive groups (carboxylic, amino, hydroxylic, etc.) that enable covalent binding to the targeting group and/or to the drug.

“On-Off” Switching (triggering): In contrast to many other diagnostic applications, TDD monitoring requires a reporter that can change its spectral properties (brightness and/or absorption/luminescence maxima and/or luminescence lifetime) upon drug release. Some switchable reporters have been recently developed, whose function is generally based on the strong change in their π-electron conjugated system upon cleavage of the trigger group (e.g., the drug).

For example, cyanine dyes were designed that contain an O substituted phenolic moiety and transform to a quinone-like structure when reacted with H₂O₂, which is overproduced during various inflammatory diseases, thus considerably increasing the fluorescence signal. The cleavable linker in these cyanines can be varied to adjust the cleavage rate. Examples of other switchable dyes, which were tested for different sensing applications, can be found, in the literature [e.g., Feng S., Fang Y., Feng W., Xia Q., Feng G. A colorimetric and ratiometric fluorescent probe with enhanced near-infrared fluorescence for selective detection of cysteine and its application in living cells. Dyes and Pigments, 2017, 146, 103-111; Fang Y., Chen W., Shi W., Li H., Xian M., Ma H. A near-infrared fluorescence off-on probe for sensitive imaging of hydrogen polysulfides in living cells and mice in vivo. Chem. Commun., 2017, 53, 8759-8762; and Ong M. J. H., Debieu S., Moreau M., Romieu A., Richard J.-A. Synthesis of N,N-dialkylamino-nor-dihydroxanthene-hemicyanine fused near-infrared fluorophores and their first water-soluble and/or bioconjugatable analogues. Chem. Asian J., 2017, 12, 936-946]. However, to date, none of these dyes has been used for TDD monitoring in a theranostic context. Moreover, this principle has never been applied to develop an activatable sensitizer whose ability to generate cytotoxic species noticeably increases in the “on” form.

The dynamic range: The magnitude of the change in luminescence intensity or sensitizing efficacy (phototoxicity) upon drug release plays an important role, as this parameter is responsible for the sensitivity, accuracy, reliability, and robustness of luminescence detection and diagnostics. A narrow dynamic range, i.e., an insufficient change in the luminescence signal upon drug release, prevents quantitative and even qualitative monitoring of drug release. The activatable sensitizers must thus significantly increase their photosensitizing efficacy.

In a luminescence-based theranostic platform, the drug is bound to the switchable luminescent dye (fluorophore) by a cleavable linker. Various external factors, such as enzymes, pH, or light, can be used to initiate the cleavage of the linker and the release of the drug. For example, a disulfide-based linker can be cleaved by endogenous thiols, including glutathione and thioredoxin, which are overexpressed in cancer cells. Ester- and carbamate-based linkers can be cleaved by esterases.

For chemotherapeutic applications, several fluorescence-based theranostic platforms have been developed to date. For instance, theranostic conjugates for TDD were studied, in which a disulfide cleavable linker was employed with several reporter dyes, including naphthalimide, coumarin, rhodol, BODIPY, and a heptamethine cyanine containing a π-conjugated hydroxylic or amino group. The same approach, based on a disulfide linker and a BODYPI reporter, was also used. Upon target-specific internalization, these conjugates undergo a thiol-triggered disulfide bond cleavage that increases the fluorescence signal of the reporter. However, in the context of theranostics, the reporters used in these studies suffer from several drawbacks that hinder their applicability. First, they are fluorescent in both the “off” and “on” forms. Although the “on” form is brighter, the dynamic range of the fluorescence change is narrow (for instance, the fluorescence intensity of BODYPI increases only by a factor of two), which limits the accurate monitoring of drug release. Second, the “on” form of BODYPI is relatively dim, which decreases the signal-to-noise ratio and, thus, the sensitivity of the measurements. Third, while BODIPY and cyanine provide excitation and fluorescence in the NIR range, naphthalimide, coumarin, and rhodol emit within the blue-green range (˜470-550 nm), and are thus not suitable for in vivo monitoring through skin and tissue. Fourth, these reporters are hydrophobic and, therefore, can aggregate in aqueous media; due to their insufficient solubility, theranostic conjugates based on these reporters were administrated intravenously, as a phosphate buffer solution containing as much as 50% DMSO, which can cause significant tissue damage.

Recently, some of the present inventors developed and investigated chemotherapeutic drug conjugates based on switchable fluorescein as the reporter [Bazylevich A., Patsenker L. D., Gellerman G. Exploiting fluorescein based drug conjugates for fluorescent monitoring in drug delivery. Dyes and Pigments, 2017, 139, 460-472]. This reporter possesses a wide dynamic range, as its fluorescence in the “off” form is almost undetectable and significantly increases in its “on” form. However, one important drawback of fluorescein is that it absorbs and emits in the blue-green region (475/516 nm), which is unsuitable for theranostic applications. Overcoming this drawback is one of the main objectives of the present invention.

For PDT applications, several theranostic platforms have been proposed, which comprise activatable (switchable) sensitizers [Lovell J. F., Liu T. W. B., Chen J., Zheng G. Activatable photosensitizers for imaging and therapy. Chem. Rev., 2010, 110, 2839-2857; and Lovell J. F., Zheng G. Activatable smart probes for molecular optical imaging and therapy. J. Innov. Opt. Health Sci., 2008, 1(1), 45-61]. These activatable sensitizers are advantageous over the conventional sensitizers because they become active (i.e., phototoxic) only upon being released in the target cells, thus reducing side-effects and improving the precision of the PDT treatment. An example of such an approach is the photochromic switchable PDT platform that is based on inorganic zirconium nanoparticles. However, an important caveat of this approach lies in the toxicity of zirconium nanoparticles and of many other types of nanoparticles. In addition, this platform is operated with blue light, which limits its applicability in deeper tissues.

Another important issue that current chemotherapeutic and PDT platforms cannot address is quantitative monitoring of drug release. The problem originates from the fact that, statistically, not all conjugate molecules accumulating in the tissue can penetrate the target cells, and not all the penetrated conjugates release the active drug. The effective ratio (R_{eff.}) describes the ratio between the number of active drug molecules (n_{drug}) and the number of conjugate molecules that accumulate in tissue sites (n_{conj}): R_{eff.}=n_{drug}/n_{conj}.

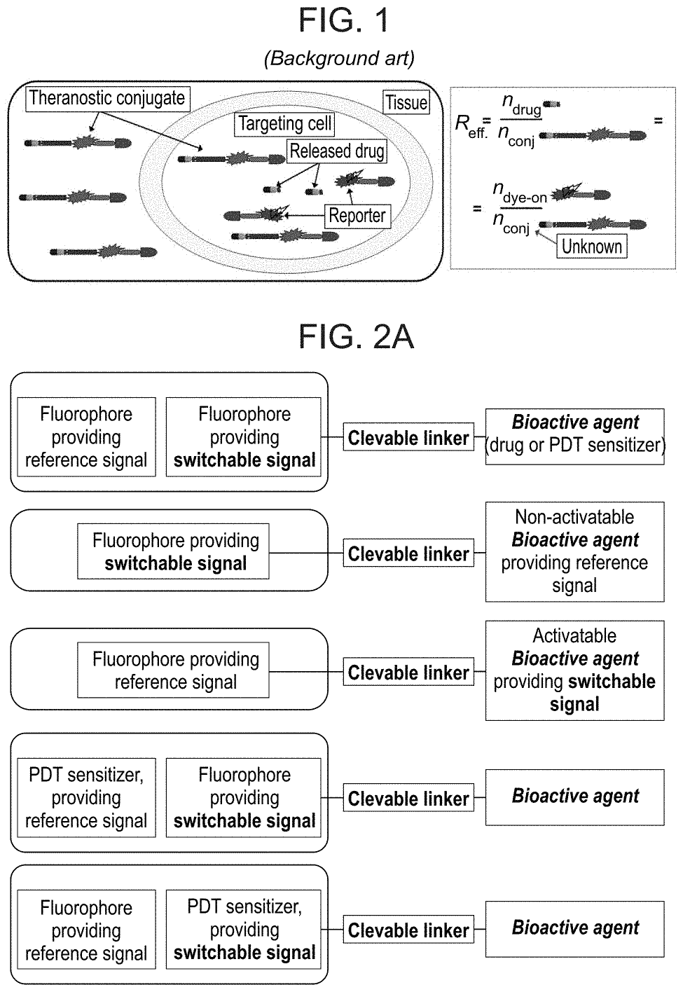

FIG. 1 presents simplified illustrations of some background art, showing theranostic conjugates and drug release in target cells, wherein “reporter” is a switchable fluorescent dye (background art).

As can be seen in FIG. 1, the number of released drug molecules (n_{drug}) is assumed to be equal to the number of reporter molecules in the “on” form (n_{dye-on}), n_{drug} is proportional to the fluorescence intensity of the switchable reporter (I_{SR}): I_{SR} ˜n_{drug}. However, n_{drug}, which is equal to n_{dye-on}, is proportional to the total number of conjugates accumulating in the tissue (n_{conj}) and, therefore, I_{SR} ˜n_{conj}×R_{eff.}. In this calculation, n_{conj} cannot be measured using current platforms because these conjugates are not fluorescent and, therefore, it is impossible to calculate the effective ratio R_{eff.}. This is a common and serious limitation of currently available theranostic platforms, and solving it is important for both clinical applications (i.e., to estimate the efficacy of TDD) and pharmaceutical research (e.g., to develop novel and highly effective TDD platforms).

While conceiving the present invention, the inventors have contemplated the conjugation of anticancer drug with cancer-specific carrier and luminescent dye, to form a theranostic system that would enable real time monitoring of targeted drug delivery (TDD). However, the luminescence signal from the dye is affected by the light absorption and scattering in body and therefore the quantitative determination of the drug release degree in target tissues is a challenging task. Therefore, the present inventors have considered ratiometric measurements utilizing two luminescent signals of different wavelengths or two luminescence lifetimes or their combination, which would improve quantitation in biological matter. The inventors aimed to prove the principle of the quantitative ratiometric luminescence monitoring of targeted drug delivery (TDD) using a dual-signal theranostic platform. The dual-signal system can be realized by two different approaches: 1) the first-type platform will comprise a “on-off” or “off-on” switchable dye (reporter or sensitizer) and a non-switchable reference reporter. The luminescence signal from the non-switchable reporter may also change upon bioactive moiety release but this change is less pronounced compared to that for the switchable dye. For this invention, it is not necessary that the signal from the non-switchable reporter must be insensitive to bioactive moiety release; it might be sensitive because of interaction with other conjugate counterparts, e.g. because of FRET, but this effect does not interfere the ratiometric measurements. Possible interference can be improved by calibration. 2) the second-type platform will comprise a dual-luminescent switchable reporter or sensitizer and a reference (non-switchable) reporter, providing two distinguishable luminescence signals before and after bioactive moiety release. While reducing the present invention to practice the present inventors have:

Synthesize and investigate switchable reporters and conjugates, “Drug—Switchable reporter”;

Designed, synthesized, and evaluated novel, dual-signal theranostic conjugates, “Drug—Switchable reporter—Reference reporter” and “Drug—Dual-luminescent Switchable reporter”, for quantitative monitoring of DD. Targeting peptide can be optionally added to the DD conjugate: Drug—Switchable reporter—Reference reporter—Targeting peptide” and “Drug—Dual-luminescent Switchable reporter—Targeting peptide”, for quantitative monitoring of TDD; Synthesized and investigated new activatable sensitizers and dual-dye PDT conjugates,

“Activatable sensitizer—Reference reporter—Targeting peptide”, with an increased dynamic range of changing the efficacy to generate cytotoxic species (singlet oxygen and/or free radicals); and

Evaluated and verified the dual-signal conjugates for the quantitative ratiometric luminescence monitoring of drug accumulation and TDD.

Thus, embodiments of the present invention combine a switchable luminescent dye (reporter or sensitizer) with a non-switchable reference reporter in the theranostic conjugate, or a single switchable reporter providing two distinguishable signals for detection in the theranostic conjugate, which will enable a quantitative, ratiometric monitoring of TDD.

While further reducing the invention to practice, a switchable, long-wavelength heptamethine cyanine dye IRD has been developed and provided herewith, which has been shown useful for ratiometric fluorescent TDD monitoring. The exemplary dye, according to some embodiments of the present invention, has been coupled to the targeting peptide octreotide amide (OctA) and, via a triggering biodegradable ester bond, has been bound to anticancer drug chlorambucil (CLB) to form a novel theranostic conjugate. The drug-bound dye absorbed and emitted light in the near-infrared (NIR) region but upon the environment-mediated drug release, its fluorescence turned red. Comparison of these two signals enabled ratiometric measurements of drug release. Advantage of the presently provided theranostic system for the ratiometric fluorescence TDD monitoring is demonstrated in the Examples section the follows below, utilizing human pancreatic cancer cell line PANC-1.

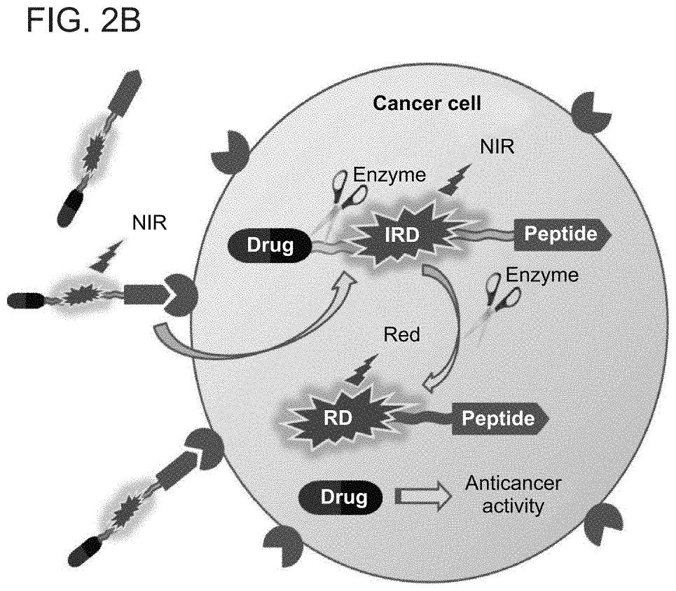

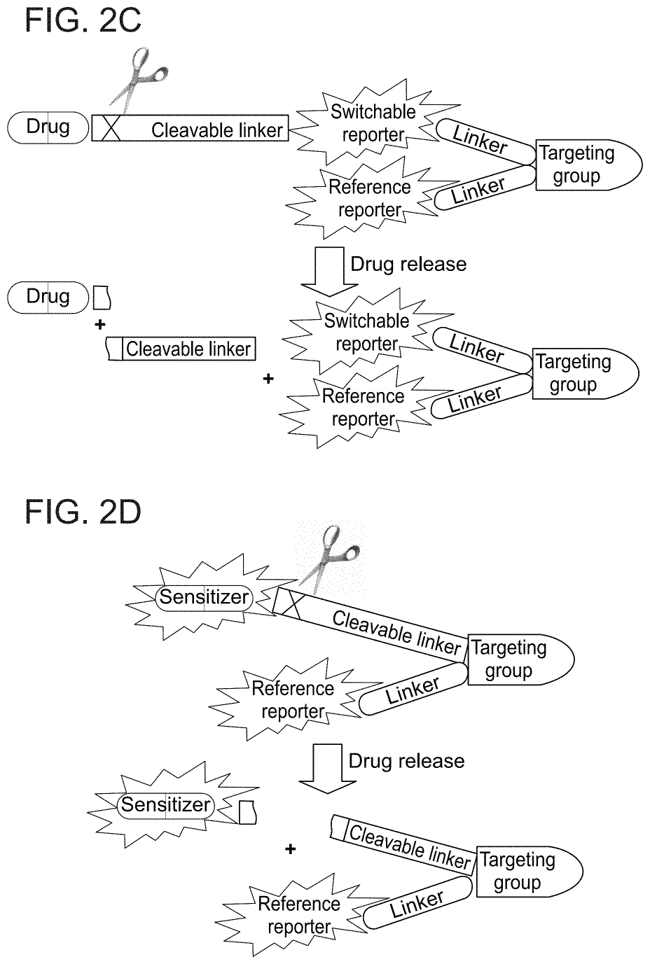

FIG. 2A-D present simplified illustrations of some embodiments of the present invention, wherein FIG. 2B shows the principle of functioning of a quantitative ratiometric fluorescence monitoring of targeted drug delivery (TDD) conjugate, according to some embodiments of the present invention, comprising a bioactive agent in the form of the anti-cancer drug CLB, a targeting moiety in the form of the peptide OctA, and a single fluorophone in the form of the switchable fluorescent dye IRD that enables ratiometric measurements, FIG. 2C presents an exemplary dual-dye (two fluorophores) theranostic conjugate for TDD of a chemotherapeutic drug, according to some embodiments of the present invention, and FIG. 2D presents an exemplary dual-dye theranostic conjugate of an activatable sensitizer.

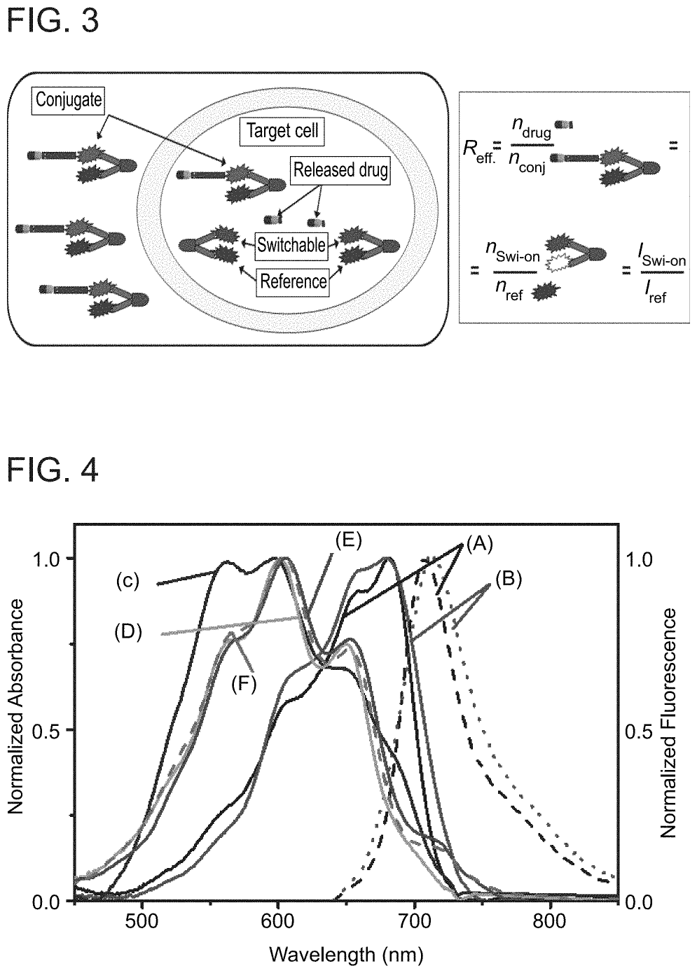

Without being bound by any particular theory, the present inventors have hypothesizes that a quantitative ratiometric fluorescence monitoring of TDD can be achieved in a dual-dye theranostic conjugate by combining two constructs: (a) a highly bright, switchable NIR dye that possesses an increased fluorescence intensity dynamic range and/or a sensitizing efficacy, and (b) a non-switchable reference reporter (FIGS. 2A-C and FIG. 3). The switchable dye is either a reporter that is sensitive to the release of a chemotherapeutic drug (FIG. 2C) or a sensitizer that is activatable upon its release from the theranostic conjugate (FIG. 2D). The switchable reporter significantly increases its fluorescence intensity when the linker is cleaved and the drug is released.

The activatable sensitizer, unlike sensitizers in general, is suggested to noticeably increase its sensitizing efficacy and fluorescence intensity when it is released from the conjugate. If the fluorescence of a sensitizer is insufficient for detection, it can be used as a “drug” with an additional switchable reporter (see FIG. 2C).

Ratiometric Theranostic Conjugate:

Ratiometric fluorescence/luminescence is the method where intensities at two or more wavelengths of an excitation or emission spectrum or two luminescence lifetimes are measured to detect changes to local environment. Typically, a probe is used that is specifically sensitive to an environmental parameter such as ion concentration, pH, viscosity, or polarity. The application of ratiometric dyes for finding probe-sensitive properties such as ion concentration can be used by measuring spectra or kinetics. Taking the ratio of the two signals directly correlates with the intracellular concentration of an analyte, the presence of which is correlated to the light signal. Measuring the ratio of the signals over time will give a time-based plot of how a solution is changing in analyte concentration. Cellular uptake of analytes is measured in this way. One of the objectives of the present invention is the provision of a ratiometric theranostic conjugate, which can be used for targeted drug delivery while allowing the caretaker to quantitatively monitor the effectiveness of the delivery mechanism.

Thus, according to an aspect of embodiments of the present invention, there is provided a conjugate, which includes:

a bioactive agent moiety,

at least one fluorophore moiety, and

a cleavable linker connecting the bioactive agent moiety and at least one of the fluorophore moieties, wherein:

the at least one fluorophore moiety is characterized by at least one reference luminescence signal and at least one switchable luminescence signal, whereas a change in the switchable luminescence signal upon cleavage of the cleavable linker is different than a change in the reference luminescence signal,

the conjugate is structured and designed so as to allow monitoring and calibrated luminescence determination of a value related to a release of the bioactive agent from the conjugate. In some embodiments, the conjugate provided herein enables quantitative determination of drug delivery in the bodily site of treatment, wherein the quantitative determination of site-directed drug release is calculated based on the dynamic recordation of the reference and switchable luminescence signals.

The term “fluorophore” or “fluorochrome”, as used herein, refers to a fluorescent chemical compound or a moiety thereof, which can emit light (luminescence, fluorescence of phosphorescence) upon light excitation (photoluminescence), chemical reaction (chemiluminescence), ultrasound (sonoluminescence) or radioactive irradiation (radioluminescence or scintillation). In this invention, any types of the above emissions can be utilized. Fluorophores typically contain several combined aromatic groups, or plane or cyclic molecules with several π bonds. In the context of embodiments of the present invention, a fluorophore moiety that exhibits a reference luminescence signal, is referred to herein as a reference fluorophore moiety, and a fluorophore moiety that exhibits a switchable luminescence signal is referred to herein as a switchable fluorophore moiety.

As used herein, the term “moiety” describes portion of a molecule, and typically a major portion thereof, or a group of atoms pertaining to a specific function.

The term “luminescence signal”, as used herein, refers to the entire set, or at least some of the measurable physical manifestations of luminescence, which characterize and can be detected in a given molecule. Manifestations of luminescence include spectral position of excitation and/or emission lines/bands, bandwidth, intensity of emission lines/bands, spectrum shape, polarization/anisotropy, lifetime and rise time of emission, and any combination, ratios of intensities of different emission bands, or product thereof, and the term “luminescence signal” therefore encompasses any one or more of the foregoing. Hence, according to some embodiments of the present invention, each of the reference luminescent signal and the switchable luminescence signal include at least one distinguishable luminescence intensity of at least one wavelengths, and/or at least one distinguishable luminescence lifetime, and/or at least one distinguishable spectrum shape, and/or at least one distinguishable bandwidth, and/or at least one distinguishable polarization/anisotropy, and any combination, ratio, product and/or correlation thereof. In some embodiments, any one of the luminescent signals include luminescence intensity, luminescence lifetime or their combination. In some embodiments, a luminescent signal is obtained from luminescence spectra, luminescence excitation spectra, luminescence synchronous spectra, luminescence lifetime, or their combination. In some embodiments, the luminescent signal includes fluorescence parameters, phosphorescence parameters, chemoluminescence parameters, and/or sonoluminescence parameters. In some embodiments, the luminescent signal is represented by one or more quantitative values stemming from the foregoing, which can be normalized or otherwise mathematically processed for comparison with another luminescent signal.

FIG. 2A presents simplified illustrations of five exemplary and non-limiting embodiments of the present invention, wherein each of the embodiments exhibits the elements of a reference fluorophore, a bioactive agent, switchable fluorophore and a cleavable linker linking the two, whereas in some embodiments, some of the elements take more than one role, and act as both a fluorophore and a bioactive agent.

In some embodiments, the conjugate includes a single fluorophore moiety, which is characterized by exhibiting two distinguishable luminescence signals—one is regarded as the reference luminescence signal and the other is regarded as a switchable luminescence signal (see, for example, FIG. 2B). This type of conjugate comprises a releasable bioactive agent, a targeting moiety (optionally), a switchable, dual-luminescent fluorophore. In this TDD conjugate, a single, switchable, dual-luminescent fluorophore is attached to the TDD conjugate. Use of this type of conjugate is based on utilizing ratiometric measurements of two signals at different wavelengths or different luminescence lifetime, which originate from the same, dual-luminescent fluorophore.

The bioactive agent release can be calculated while accounting for that one of the signals decreases while the second one increases; the first one is proportional to the concentration of the bound bioactive agent, while the second one indicates the concentration of free (released) bioactive agent. Using this type of conjugates, the efficacy of drug delivery (R_{eff.}) can be quantified as the ratio between the fluorescent signal corresponding to conjugate with the cleaved linker (I_{Swi-on}) and the fluorescent signal corresponding to conjugate with the non-cleaved linker (I_{ref.}): R_{eff.}=k×(I_{Swi-on}/I_{ref.}) (see, FIG. 3), where k is a calibration coefficient that can be calculated or determined experimentally.

In some embodiments, the conjugate includes at least two fluorophore moieties, one of which is characterized by a distinguishable reference luminescence signal, and another is characterized by a distinguishable switchable luminescence (see, for example, FIGS. 2B-C and FIG. 3). This type of conjugate comprises releasable bioactive agent, a targeting moiety (optionally), a switchable fluorophore and a non-switchable fluorophore. Use of this type of conjugate is based on utilizing the presence of both switchable and non-switchable (reference) fluorophores attached simultaneously to the TDD conjugate. Using this type of conjugates, the efficacy of drug delivery (R_{eff.}) can be quantified as the ratio between the number of drug molecules released in the target cell interior (n_{drug}) and the total number of theranostic conjugates (n_{conj}) delivered in the treated tissue sites (inside and outside the target cells): R_{eff.}=n_{drug}/n_{conj}. This ratio can be calculated by ratiometric measurements using a reference non-switchable reporter: R_{eff.}=n_{Swi-on}/n_{ref.}=k×(I_{Swi-on}/I_{ref.}) (see, FIG. 3), where n_{Swi-on} and n_{ref.} are the number of switchable dye molecules in the “on” form and the number of reference reporter molecules, respectively, and I_{Swi-on} and I_{ref.} are their fluorescence intensities; k is a calibration coefficient that can be calculated or determined experimentally.

An activatable sensitizer is contemplated as one of the uses of the conjugate provided herein. Such activatable sensitizer is expected to noticeably increase its sensitizing efficacy and fluorescence intensity (for monitoring applications) when it is released from the conjugate at the targeted site. If the luminescence signal of a sensitizer is insufficient for detection, it can be used as a “drug” with an additional switchable reporter (FIG. 2C). If the luminescence signal of a sensitizer is sufficient for detection, the conjugate can be utilized as presented in FIG. 2D. A conjugate that includes a PDT sensitizer can be used as an activatable sensitizer; it comprises an activatable and non-switchable reference reporter for ratiometric luminescence monitoring of sensitizer release, wherein upon its release the sensitizer (bioactive agent) becomes active, begins to generate reactive cytotoxic species, and provides luminescent signal for detection.

In the context of some embodiments of the present invention, a switchable luminescence signal is influenced by cleavage of the cleavable linker, and a reference luminescence signal is essential uninfluenced by this cleavage. In order to afford a ratiometric conjugate, according to some embodiment if the present invention, the conjugate should exhibit the two distinct types of luminescence signals, and the distinction between the two types is based on the degree of influence of said cleavage on the luminescence signal, or the extent of the change in the luminescence signal as observed upon said cleavage. According to some embodiments, the change in the switchable luminescence signal is at least 10% greater than the change in said reference luminescence signal, as can be reckoned from the quantitative values representing each of the compared luminescence signals.

In order for the conjugate provided herein to be useful in the context of diagnostic treatment of a living organism, such as a mammal, the luminescence signals exhibited thereby are selected to be distinguishable within the wavelength range of 600 nm to 900 nm. Alternatively, the wavelength range for effective use of the conjugate depends on the nature (structure) of the fluorophore(s), the size of the treated area/organ/tissue, the distance thereof from the accessible surface of the body, the probe/detector/machinery used to detect the signals, and the likes.

In some embodiments, the conjugate provided herein is designed and constructed so as to allow theranostic bioavailability at physiological conditions. In other words, the conjugate is synthesized so as to exhibit sufficient solubility in physiological media, which is afforded by introducing or removing certain groups and moieties in the conjugate, that affect its bioavailability, as these parameters and synthetic procedures are known to and are well within the capacity of a skilled artisan.

Fluorophores:

As discussed hereinabove, the conjugate provided herein includes one or more fluorophore (reporter) moieties, which are used in order for the conjugate to emit two types of luminescence signals, a switchable luminescence signal and a reference luminescence signal, which are notably distinguishable in the extent they are affected by the cleavage of the cleavable linker. The selection of fluorophore moieties should take into account the location of the fluorophore moiety in the conjugate (directly or indirectly attached to the cleavable linker), and minimize spectral overlap; in addition, fluorophore (reporter) moieties emitting in the NIR region are preferable as fluorophores for both the reference and switchable luminescence signal.

In some embodiments, the at least one fluorophore moiety that exhibits both a reference luminescence signal and a switchable luminescence signal (dual-signal fluorophores), is fluorophore moiety selected from the group consisting of:

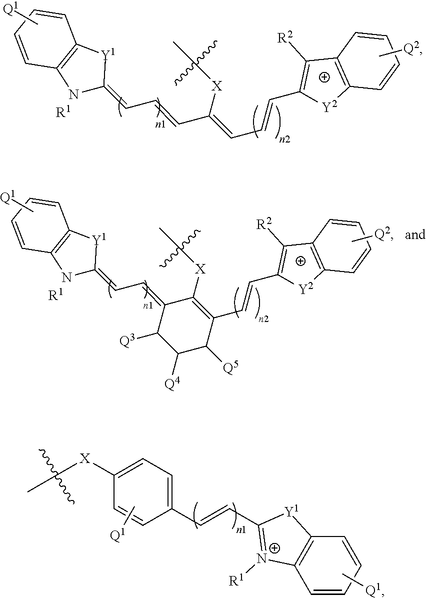

wherein:

X═O, S, Se, NR^{N}, 2-phenyoxy, 4-phenyoxy, aryloxy;

R^{N}=hydrogen, alkyl, aryl alkylaryl, or contain a reactive group or a solubilizing group selected from sulfate, sulphonate, quaternary amine, phosphate, phosphonate and PEG;

Y¹, Y² are independently selected from C(R^{a}, R^{b}), O, S, NR^{N};

R^{a}, R^{b} are independently selected from hydrogen, alkyl, aryl alkylaryl, a contain a reactive group or solubilizing group selected from sulfate, sulphonate, quaternary amine, phosphate, phosphonate and PEG;

R^{a} and R^{b} can form a ring;

R¹, R² are independently selected from hydrogen, alkyl, aryl alkylaryl, or contain a reactive group or a solubilizing group selected from sulfate, sulphonate, quaternary amine, phosphate, phosphonate and PEG;

Q¹, Q² are at least one of groups consisting of R¹, halogen, cyano, sulfo, phosphate, carboxy, formyl, alkyl, aryl, alkylaryl, alkoxy, aryloxy or a substituted or unsubstituted cyclic moiety; two adjacent Q¹ and two adjacent Q² can form a substituted or unsubstituted cyclic moiety;

each of

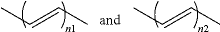

is independently a linear or cyclic, substituted or unsubstituted polyene, and each of n1 and n2 is independently an integer ranging 1-4; and

the wiggled line represents attachment to said cleavable linker.

Non-limiting examples of such dual-signal fluorophores include a heptamethine cyanine, and 2-((E)-2-((E)-3-((Z)-2-(3-(5-carboxypentyl)-1,1-dimethyl-1H-inden-2(3H)-ylidene)ethylidene)-2-hydroxycyclohex-1-en-1-yl)vinyl)-1,3,3-trimethyl-3H-indol-1-ium.

According to some embodiments, the conjugate includes a switchable fluorophore moiety that emits the switchable luminescent signal, is fluorophore moiety selected from the group consisting of:

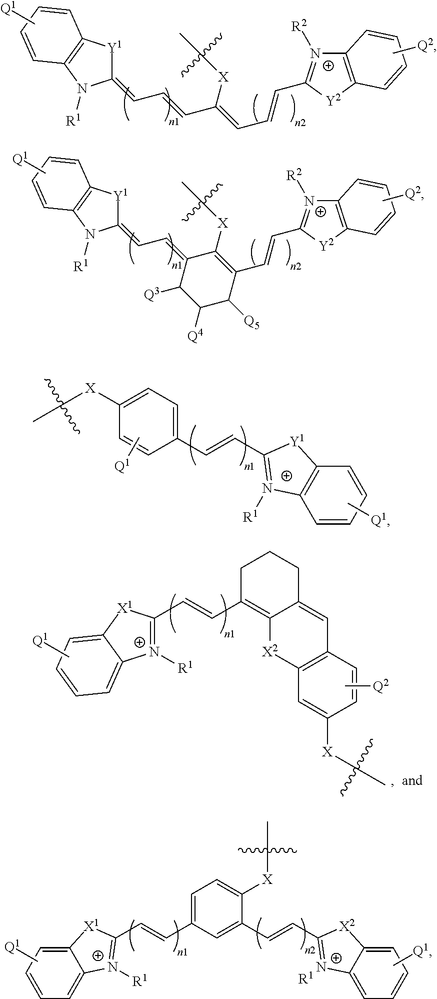

wherein:

X═O, S, Se, NR^{N}, 2-phenyoxy, 4-phenyoxy, aryloxy;

R^{N}=hydrogen, alkyl, aryl alkylaryl, or contain a reactive group or a solubilizing group selected from sulfate, sulphonate, quaternary amine, phosphate, phosphonate and PEG;

Y¹, Y² are independently selected from C(R^{a}, R^{b}), O, S, NR^{N};

R^{a}, R^{b} are independently selected from hydrogen, alkyl, aryl alkylaryl, or contain a reactive group or a solubilizing group selected from sulfate, sulphonate, quaternary amine, phosphate, phosphonate, PEG;

R^{a} and R^{b} can form a ring;

R¹, R² are each independently selected from hydrogen, alkyl, aryl alkylaryl, or contain a reactive group or a solubilizing group selected from sulfate, sulphonate, quaternary amine, phosphate, phosphonate, PEG;

Q¹, Q² are at least one of groups consisting of R¹, halogen, cyano, sulfo, phosphate, carboxy, formyl, alkyl, aryl, alkylaryl, alkoxy, aryloxy or a substituted or unsubstituted cyclic moiety; two adjacent Q¹ and two adjacent Q² can form a substituted or unsubstituted cyclic moiety;

each of

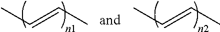

is independently a linear or cyclic, substituted or unsubstituted polyene, and each of n1 and n2 is independently an integer ranging 1-4; the wiggled line represents attachment to said cleavable linker

A fluorophore moiety that emits a switchable luminescent signal, is selected from the group consisting of a phenolic cyanine or styryl dye, a 2,3-dihydro-1H-xanthen-6-ol cyanine or styryl dye, a 7-hydroxynaphthalen-2(1H)-one cyanine or styryl dye, and a 6-hydroxyquinolin-3(4H)-one cyanine or styryl dye.

According to some embodiments, the conjugate includes a reference fluorophore moiety, which is a moiety of a fluorescent dye selected from the group consisting of a cyanine-based fluorescent dye, a styryl-based fluorescent dye, a squaraine-based fluorescent dye, a squaraine-rotaxane-based fluorescent dye, a phthalocyanine-based fluorescent dye, a porphyrine-based fluorescent dye, a xanthene-based dye, a phenothiazine-based dye, a luminescent metal-ligand complex, a fluorescent protein, a luminescent nanoparticle, a luminescent quantum dot, a luminescent nanocrystal, a luminescent polymeric particle, a tandem fluorophore, or a fluorescent dye selected from Cy, Dy, Alexa Fluor, IRDye, LiCor, BODIPY, SETA dye series.

Structural Elements in the Conjugate:

As used herein, the words “link”, “linked”, “linkage” “linker”, “bound”, “coupled” or “attached”, are used interchangeably herein and refer to the presence of at least one covalent bond between species and moieties, unless specifically noted otherwise.

As used herein, the term “linking moiety” describes a chemical moiety (a group of atoms or a covalent bond) that links two chemical moieties via one or more covalent bonds. A linking moiety may include atoms that form a part of one or both of the chemical moieties it links, and/or include atoms that do not form a part of one or both of the chemical moieties it links. For example, a peptide bond (amide) linking moiety that links two amino acids includes at least a nitrogen atom and a hydrogen atom from one amino acid and at least a carboxyl of the other amino acid. In general, the linking moiety can be formed during a chemical reaction, such that by reacting two or more reactive groups, the linking moiety is formed as a new chemical entity which can comprise a bond (between two atoms), or one or more bonded atoms. Alternatively, the linking moiety can be an independent chemical moiety comprising two or more reactive groups to which the reactive groups of other compounds can be attached, either directly or indirectly, as is detailed hereinunder.

In the context of some embodiments of the present invention, the term “linking moiety” is synonymous with the term “cleavable linker”, meaning that the linking moiety is selected or designed to break under certain conditions, or at certain locations in the treated subject.

The positions at which the bioactive agent is linked to the conjugate presented herein are generally selected such that once cleaved off the conjugate, any remaining moiety stemming from the linking moiety (or a spacer moiety) on the bioactive agent, if at all, does not substantially preclude its biological activity (mechanism of biological activity). Suitable positions depend on the type of bioactive agent and cleavable linker. According to some embodiments of the present invention, the linking moieties are form such that the biological activity of the bioactive agent, once released from the conjugate, is not abolished and remains substantially the same as the biological activity of a similar pristine bioactive agent. It is noted that the bioactive agent, as long as it is bound to the conjugate, can be regarded as a prodrug, which upon its release from the conjugate, is in its bioactive form.

In some embodiments, the term “linking moiety” encompasses an amino acid residue, or a peptide of two or more amino acids residues. In such embodiments, the conjugate may be regarded as one that comprises one or more amino acid residues that do not bear a bioactive agent. In some embodiments, the term “linking moiety” is defined so as not to encompass an amino acid residue or a peptide. In such embodiments, the conjugate may be regarded as one that does not include amino acid residues that do not bear at least one bioactive agent.

The phrase “reactive group”, as used herein, refers to a chemical group that is capable of undergoing a chemical reaction that typically leads to the formation a covalent bond. Chemical reactions that lead to a bond formation include, for example, cycloaddition reactions (such as the Diels-Alder's reaction, the 1,3-dipolar cycloaddition Huisgen reaction, and the similar “click reaction”), condensations, nucleophilic and electrophilic addition reactions, nucleophilic and electrophilic substitutions, addition and elimination reactions, alkylation reactions, rearrangement reactions and any other known organic reactions that involve a reactive group.

Representative examples of reactive groups include, without limitation, acyl halide, aldehyde, alkoxy, alkyne, amide, amine, aryloxy, azide, aziridine, azo, carbamate, carbonyl, carboxyl, carboxylate, cyano, diene, dienophile, epoxy, guanidine, guanyl, halide, hydrazide, hydrazine, hydroxy, hydroxylamine, imino, isocyanate, isothiocyanate, maleimide, N-hydroxycuccinimide, carboxylic acid halide, alkyl halide, nitro, phosphate, phosphonate, sulfinyl, sulfonamide, sulfonate, thioalkoxy, thioaryloxy, thiocarbamate, thiocarbonyl, thiohydroxy, thiourea and urea, as these terms are defined hereinafter.

According some embodiments of the present invention, various elements of the conjugate presented herein are attached to one or more linking moieties via spacer moieties. As used herein, the phrase “spacer moiety” describes a chemical moiety that typically extends between two chemical moieties and is attached to each of the chemical moieties via covalent bonds. The spacer moiety may be linear or cyclic, be branched or unbranched, rigid or flexible, hydrophobic or hydrophilic.

The nature of the spacer moieties can be regarded as having an effect on two aspects, the synthetic aspect, namely the influence of the spacer moieties on the process of preparing the conjugates presented herein, and the influence of the spacer moieties on the biology activity of the conjugates in terms of drug-release profile(s), biological activity, bioavailability and other ADME-Tox considerations.

According to some embodiments of the present invention, the spacer moieties are selected such that they allow and/or promote the conjugation reaction between various elements of the conjugates presented herein, and reduce the probability for the formation of side-products due to undesired reactions. Such traits can be selected for in terms of spacer's length, flexibility, structure and specific chemical reactivity or lack thereof. Spacer moieties with fewer reactive groups will present a simpler synthetic challenge, requiring less protection/deprotection steps and affording higher chemical yields. For example, saturated and linear alkyls of 1-10, or 1-5 carbon atoms, having one reactive group at the end atom for conjugation with a corresponding reactive group, would afford substantially higher yield and fewer side products. Similarly, a spacer moiety based on one or two chained benzyl rings would also lead to an efficient conjugation reaction.

According to some embodiments of the present invention, the spacer moieties are selected such that they provide favorable cleavage conditions, as these are discussed hereinbelow. For example, a spacer may alter the accessibility of an enzyme to the linking moiety, thereby allowing the enzyme to cleave the linkage between the bioactive agent and the conjugate.

According to some embodiments of the present invention, the spacer moieties include, without limitation, —CH₂—, —CH₂—O—, —(CH₂)₂—, —(CH₂)₂—O—, —(CH₂)₃—, —(CH₂)₃—O—, —(CH₂)₄—, —(CH₂)₅—, —(CH₂)₆—, —(CH(CH₃))—CH₂—, —CH═CH—CH═CH—, —C≡C—C≡C—, —CH₂CH(OH)CH₂—, —CH₂—O—CH₂—, —CH₂—O—CH₂—O—, —(CH₂)₂—O—(CH₂)₂—, —(CH₂)₂—O—(CH₂)₂—O—, —CH₂-mC₆H₄—CH₂—, —CH₂-mC₆H₄—CH₂—O—, —CH₂-pC₆H₄—CH₂—, —CH₂-pC₆H₄—CH₂—O—, —CH₂—NHCO—, —C₆H₄—NHCO—, —CH₂—O—CH₂— and —CH═CH—CH₂—NH—(CH₂)₂—.

In some embodiments, the spacer is a moiety having more than two reactive functionalities (reactive groups) that can be utilized to tether other elements of the conjugate, thereby forming the core of the conjugate. For example, the amino-acid lysine has three reactive groups in the form of the α-amine, the α-carboxyl, and the amine at the end of the side-chain; hence, a lysine residue can be used to tether the fluorophore moieties and the targeting moiety into a single molecular entity, constituting the conjugate provided herein. For a demonstration of the above, see, Example 6 in the Examples section hereinbelow.

In some embodiments, a spacer moiety can be regarded as forming a part of a linking moiety.

Examples of linking moieties, according to some embodiments of the present invention, include without limitation, amide, carbamate, carbonate, lactone, lactam, carboxylate, ester, cycloalkene, cyclohexene, heteroalicyclic, heteroaryl, triazine, triazole, disulfide, imine, imide, oxime, aldimine, ketimine, hydrazone, semicarbazone, acetal, ketal, aminal, aminoacetal, thioacetal, thioketal, phosphate ester, and the like. Other linking moieties are defined hereinbelow, and further other linking moieties are contemplated within the scope of the term as used herein.

According to some embodiments, the cleavable linker, or labile linking moiety, is selected from the group consisting of:

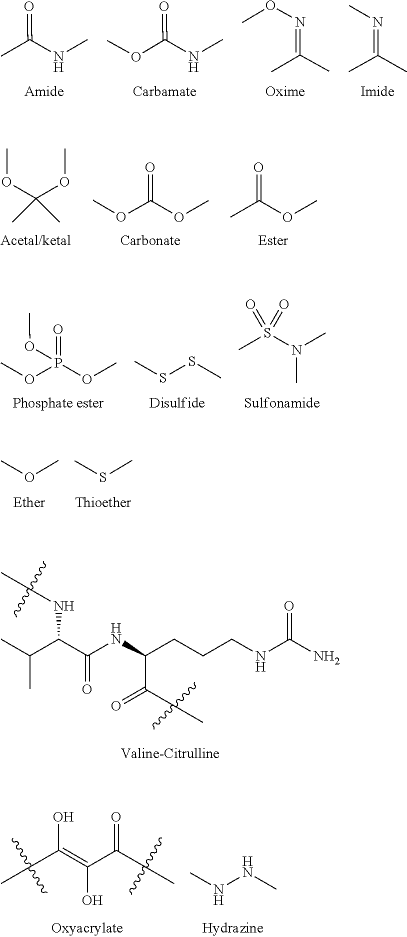

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry. Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

As used herein, the terms “amine” or “amino”, describe both a —NR′R″ end group and a —NR′— linking moiety, wherein R′ and R″ are each independently hydrogen, alkyl, cycloalkyl, aryl, as these terms are defined hereinbelow.

Herein throughout, the phrase “end group” describes a chemical group that is attached to one compound (a substituent; a reactive group; a functional group etc.), while the term “linking moiety” describes a group that is attached to two compounds and links therebetween.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydrogen, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine, as these terms are defined herein.

The term “alkyl” describes a saturated aliphatic hydrocarbon including straight chain (unbranched) and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking moiety, as this phrase is defined hereinabove, which connects two or more moieties via at least two carbons in its chain. When an alkyl is a linking moiety, it is also referred to herein as “alkylene”, e.g., methylene, ethylene, propylene, etc. The term “alkenyl” describes an unsaturated alkyl, as defined herein, having at least two carbon atoms and at least one carbon-carbon double bond. The alkenyl may be substituted or unsubstituted by one or more substituents, as described for alkyl hereinabove.

The terms “alkynyl” or “alkyne”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings that share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking moiety, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking moiety, as this phrase is defined hereinabove, connecting two or more moieties at two or more positions thereof. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking moiety, as this term is defined hereinabove, connecting two or more moieties at two or more positions thereof. Preferably, the aryl is phenyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking moiety, as this phrase is defined hereinabove, connecting two or more moieties at two or more positions thereof. Representative examples are pyridine, pyrrole, oxazole, indole, purine and the like.

The term “alkaryl” describes an alkyl, as defined herein, which is substituted by one or more aryl or heteroaryl groups. An example of alkaryl is benzyl.

The term “amine-oxide” describes a —N(OR′)(R″) or a —N(OR′)— group, where R′ and R″ are as defined herein. This term refers to a —N(OR′)(R″) group in cases where the amine-oxide is an end group, as this phrase is defined hereinabove, and to a —N(OR′)— group in cases where the amine-oxime is an end group, as this phrase is defined hereinabove.

As used herein, the term “acyl” refers to a group having the general formula —C(═O)R′, —C(═O)OR′, —C(═O)—O—C(═O)R′, —C(═O)SR′, —C(═O)N(R′)₂, —C(═S)R′, —C(═S)N(R′)₂, and —C(═S)S(R′), —C(═NR′)R″, —C(═NR′) OR″, —C(═NR′)SR″, and —C(═NR′)N(R″)₂, wherein R′ and R″ are each independently hydrogen, halo, substituted or unsubstituted hydroxyl, substituted or unsubstituted thiol, substituted or unsubstituted amine, substituted or unsubstituted acyl, cyclic or acyclic, substituted or unsubstituted, branched or unbranched aliphatic, cyclic or acyclic, substituted or unsubstituted, branched or unbranched heteroaliphatic, cyclic or acyclic, substituted or unsubstituted, branched or unbranched alkyl, cyclic or acyclic, substituted or unsubstituted, branched or unbranched alkenyl, substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thioxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “aliphatic” or “aliphatic group” denotes an optionally substituted hydrocarbon moiety that may be straight-chain (i.e., unbranched), branched, or cyclic (“carbocyclic”) and may be completely saturated or may contain one or more units of unsaturation, but which is not aromatic. Unless otherwise specified, aliphatic groups contain 1-12 carbon atoms. In some embodiments, aliphatic groups contain 1-6 carbon atoms. In some embodiments, aliphatic groups contain 1-4 carbon atoms, and in yet other embodiments, aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

As used herein, the terms “heteroaliphatic” or “heteroaliphatic group”, denote an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. Unless otherwise specified, heteroaliphatic groups contain 1-6 carbon atoms wherein 1-3 carbon atoms are optionally and independently replaced with heteroatoms selected from oxygen, nitrogen and sulfur. In some embodiments, heteroaliphatic groups contain 1-4 carbon atoms, wherein 1-2 carbon atoms are optionally and independently replaced with heteroatoms selected from oxygen, nitrogen and sulfur. In yet other embodiments, heteroaliphatic groups contain 1-3 carbon atoms, wherein 1 carbon atom is optionally and independently replaced with a heteroatom selected from oxygen, nitrogen and sulfur. Suitable heteroaliphatic groups include, but are not limited to, linear or branched, heteroalkyl, heteroalkenyl, and heteroalkynyl groups.

The term “halo” describes fluorine, chlorine, bromine or iodine substituent.

The term “halide” describes an anion of a halogen atom, namely F⁻, Cl⁻ Br⁻ and I⁻.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking moiety, as these phrases are defined hereinabove, where R′ is as defined hereinabove.

The term “thiosulfate” describes a —O—S(═S)(═O)—OR′ end group or a —O—S(═S)(═O)—O— linking moiety, as these phrases are defined hereinabove, where R′ is as defined hereinabove.

The term “sulfite” describes an —O—S(═O)—O—R′ end group or a —O—S(═O)—O—group linking moiety, as these phrases are defined hereinabove, where R′ is as defined hereinabove.

The term “thiosulfite” describes a —O—S(═S)—O—R′ end group or an —O—S(═S)—O—group linking moiety, as these phrases are defined hereinabove, where R′ is as defined hereinabove.

The term “sulfinate” or “sulfinyl” describes a —S(═O)—OR′ end group or an —S(═O)—O—group linking moiety, as these phrases are defined hereinabove, where R′ is as defined hereinabove.

The terms “solfoxide” or “sulfinyl” describe a —S(═O)R′ end group or an —S(═O)—linking moiety, as these phrases are defined hereinabove, where R′ is as defined hereinabove.

The term “sulfonate” or “sulfonyl” describes a —S(═O)₂—R′ end group or an —S(═O)₂— linking moiety, as these phrases are defined hereinabove, where R′ is as defined herein.

The term “S-sulfonamide” describes a —S(═O)₂—NR′R″ end group or a —S(═O)₂—NR′— linking moiety, as these phrases are defined hereinabove, with R′ and R″ as defined herein.

The term “N-sulfonamide” describes an R′S(═O)₂—NR″— end group or a —S(═O)₂—NR′— linking moiety, as these phrases are defined hereinabove, where R′ and R″ are as defined herein.

The term “disulfide” refers to a —S—SR′ end group or a —S—S— linking moiety, as these phrases are defined hereinabove, where R′ is as defined herein.

The term “phosphate” describes an —O—P(═O)₂(OR′) end or reactive group or a —O—P(═O)₂(O)— linking moiety, as these phrases are defined hereinabove, with R′ as defined herein.

The term “phosphonate” describes a —P(═O)(OR′)(OR″) end or reactive group or a —P(═O)(OR′)(O)— linking moiety, as these phrases are defined hereinabove, with R′ and R″ as defined herein.

The term “thiophosphonate” describes a —P(═S)(OR′)(OR″) end group or a —P(═S)(OR′)(O)— linking moiety, as these phrases are defined hereinabove, with R′ and R″ as defined herein.

The term “carbonyl” or “carbonate” as used herein, describes a —C(═O)—R′ end group or a —C(═O)— linking moiety, as these phrases are defined hereinabove, with R′ as defined herein.

The term “thiocarbonyl” as used herein, describes a —C(═S)—R′ end group or a —C(═S)— linking moiety, as these phrases are defined hereinabove, with R′ as defined herein.

The term “oxo” as used herein, described a ═O end group.

The term “thioxo” as used herein, described a ═S end group.

The term “oxime” describes a ═N—OH end group or a ═N—O— linking moiety, as these phrases are defined hereinabove.

The term “hydroxyl” describes a —OH group.

As used herein, the term “aldehyde” refers to an —C(═O)—H group.

The term “acyl halide” describes a —(C=O)R″″ group wherein R″″ is halo, as defined hereinabove.

The term “alkoxy” as used herein describes an —O-alkyl, an —O-cycloalkyl, as defined hereinabove. The ether group —O—is also a possible linking moiety.

The term “aryloxy” describes both an —O-aryl and an —O-heteroaryl group, as defined herein.

The term “disulfide” as used herein describes an —S—S— linking moiety, which in some cases forms between two thiohydroxyl groups.

The terms “thio”, “sulfhydryl” or “thiohydroxyl” as used herein describe an —SH group.

The term “thioalkoxy” or “thioether” describes both a —S-alkyl group, and a —S-cycloalkyl group, as defined herein. The thioether group —S— is also a possible linking moiety.

The term “thioaryloxy” describes both a —S-aryl and a —S-heteroaryl group, as defined herein. The thioarylether group —S—aryl- is also a possible linking moiety.

The term “cyano” or “nitrile” describes a —C≡N group.

The term “isocyanate” describes an —N═C═O group.

The term “nitro” describes an —NO₂ group.

The term “carboxylate” or “ester”, as used herein encompasses C-carboxylate and O-carboxylate.

The term “C-carboxylate” describes a —C(═O)—OR′ end group or a —C(═O)—O—linking moiety, as these phrases are defined hereinabove, where R′ is as defined herein.

The term “O-carboxylate” describes a —OC(═O)R′ end group or a —OC(═O)— linking moiety, as these phrases are defined hereinabove, where R′ is as defined herein.

The term “thiocarboxylate” as used herein encompasses “C-thiocarboxylate and O-thiocarboxylate.

The term “C-thiocarboxylate” describes a —C(═S)—OR′ end group or a —C(═S)—O—linking moiety, as these phrases are defined hereinabove, where R′ is as defined herein.

The term “O-thiocarboxylate” describes a —OC(═S)R′ end group or a —OC(═S)— linking moiety, as these phrases are defined hereinabove, where R′ is as defined herein.

The term “carbamate” as used herein encompasses N-carbamate and O-carbamate.

The term “N-carbamate” describes an R″OC(═O)—NR′—end group or a —OC(═O)—NR′—linking moiety, as these phrases are defined hereinabove, with R′ and R″ as defined herein.

The term “O-carbamate” describes an —OC(═O)—NR′R″ end group or an —OC(═O)—NR′—linking moiety, as these phrases are defined hereinabove, with R′ and R″ as defined herein.

The term “thiocarbamate” as used herein encompasses N-thiocarbamate and O-thiocarbamate.

The term “O-thiocarbamate” describes a —OC(═S)—NR′R″ end group or a —OC(═S)—NR′—linking moiety, as these phrases are defined hereinabove, with R′ and R″ as defined herein.

The term “N-thiocarbamate” describes an R″OC(═S)NR′— end group or a —OC(═S)NR′— linking moiety, as these phrases are defined hereinabove, with R′ and R″ as defined herein. The term “dithiocarbamate” as used herein encompasses N-dithiocarbamate and S-dithiocarbamate.

The term “S-dithiocarbamate” describes a —SC(═S)—NR′R″ end group or a —SC(═S)NR′— linking moiety, as these phrases are defined hereinabove, with R′ and R″ as defined herein.

The term “N-dithiocarbamate” describes an R″SC(═S)NR′— end group or a —SC(═S)NR′— linking moiety, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking moiety, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R″″ end group or a —NR′—C(═S)—NR″—linking moiety, with R′, R″ and R′″ as defined herein.

The term “amide” as used herein encompasses C-amide and N-amide.

The term “C-amide” describes a —C(═O)—NR′R″ end group or a —C(═O)—NR′—linking moiety, as these phrases are defined hereinabove, where R′ and R″ are as defined herein.

The term “N-amide” describes a R′C(═O)—NR″—end group or a R′C(═O)—N—linking moiety, as these phrases are defined hereinabove, where R′ and R″ are as defined herein.

The term “imine”, which is also referred to in the art interchangeably as “Schiff-base”, describes a —N═CR′— linking moiety, with R′ as defined herein or hydrogen. As is well known in the art, Schiff bases are typically formed by reacting an aldehyde or a ketone and an amine-containing moiety such as amine, hydrazine, hydrazide and the like, as these terms are defined herein. The term “aldimine” refers to a —CH═N— imine which is derived from an aldehyde. The term “ketimine” refers to a —CR′=N—imine which is derived from a ketone.

The term “hydrazone” refers to a —R′C=N—NR″— linking moiety, wherein R′ and R″ are as defined herein.

The term “semicarbazone” refers to a linking moiety which forms in a condensation reaction between an aldehyde or ketone and semicarbazide. A semicarbazone linking moiety stemming from a ketone is a —R′C═NNR″C(═O)NR′″—, and a linking moiety stemming from an aldehyde is a —CR′=NNR″C(═O)NR′″—, wherein R′ and R″ are as defined herein and R′″ or as defined for R′.

As used herein, the term “lactone” refers to a cyclic ester, namely the intra-condensation product of an alcohol group —OH and a carboxylic acid group —COOH in the same molecule.

As used herein, the term “lactam” refers to a cyclic amide, as this term is defined herein. A lactam with two carbon atoms beside the carbonyl and four ring atoms in total is referred to as a β-lactam, a lactam with three carbon atoms beside the carbonyl and five ring atoms in total is referred to as a γ-lactam, a lactam with four carbon atoms beside the carbonyl and six ring atoms in total is referred to as a δ-lactam, and so on.

The term “guanyl” describes a R′R″NC(═N)— end group or a —R′NC(═N)— linking moiety, as these phrases are defined hereinabove, where R′ and R″ are as defined herein.

The term “guanidine” describes a —R′NC(═N)—NR″R″″ end group or a —R′NC(═N)—NR″— linking moiety, as these phrases are defined hereinabove, where R′, R″ and R′″ are as defined herein.

The term “hydrazine” describes a —NR′—NR″R″″ end group or a —NR′—NR″— linking moiety, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking moiety, as these phrases are defined hereinabove, where R′, R″ and R′″ are as defined herein.

The term “hydroxylamine”, as used herein, refers to either a —NHOH group or a —ONH₂. As used herein, the terms “azo” or “diazo” describe a —N═N—R′ end group or a —N═N— linking moiety, as these phrases are defined hereinabove, where R′ is as defined herein.

As used herein, the term “azido” described a —N═N⁺═N⁻ (—N₃) end group.

The term “triazine” refers to a heterocyclic ring, analogous to the six-membered benzene ring but with three carbons replaced by nitrogen atoms. The three isomers of triazine are distinguished from each other by the positions of their nitrogen atoms, and are referred to as 1,2,3-triazine, 1,2,4-triazine, and 1,3,5-triazine. Other aromatic nitrogen heterocycles include pyridines with 1 ring nitrogen atom, diazines with 2 nitrogen atoms in the ring and tetrazines with 4 ring nitrogen atoms.

The term “triazole” refers to either one of a pair of isomeric chemical compounds with molecular formula C₂H₃N₃, having a five-membered ring of two carbon atoms and three nitrogen atoms, namely 1,2,3-triazoles and 1,2,4-triazoles.

The term “aziridine”, as used herein, refers to a reactive group which is a three membered heterocycle with one amine group and two methylene groups, having a molecular formula of —C₂H₃NH.

As used herein, the term “thiohydrazide” describes a —C(═S)—NR′—NR″R′″ end group or a —C(═S)—NR′—NR″— linking moiety, as these phrases are defined hereinabove, where R′, R″ and R′″ are as defined herein.

As used herein, the term “methyleneamine” describes an —NR′—CH₂—CH═CR″R′″ end group or a —NR′—CH₂—CH═CR″— linking moiety, as these phrases are defined hereinabove, where

R′, R″ and R′″ are as defined herein.

The term “diene”, as used herein, refers to a —CR′=CR″—CR′″=CR″″— group, wherein R′ as defined hereinabove, and R″, R′″ and R″″ are as defined for R′.

The term “dienophile”, as used herein, refers to a reactive group that reacts with a diene, typically in a Diels-Alder reaction mechanism, hence a dienophile is typically a double bond or an alkenyl.

The term “epoxy”, as used herein, refers to a reactive group which is a three membered heterocycle with one oxygen and two methylene groups, having a molecular formula of —C₂H₃O.

The phrase “covalent bond”, as used herein, refers to one or more pairs of electrons that are shared between atoms in a form of chemical bonding.

According to some embodiments of the present invention, some linking moieties result from a reaction between two reactive groups. Alternatively, a desired linking moiety is first generated and a bioactive agent and/or a spacer moiety are attached thereto.

Cleavable Linker Lability:

According to some embodiments of the present invention, a linking moiety may be stable at physiological conditions, namely the linking moiety does not disintegrate for the duration of exposure to the physiological environment in the bodily site. Such linking moiety is referred to herein a “biostable”. Biostable linking moieties offer the advantage of an extended period of time at which the conjugate can exert its biological activity (releasing bioactive agents at the targeted bodily site), up to the time it is secreted or otherwise removed from the bodily site. An exemplary biostable linking moiety is a triazole-based linking moiety. It is noted that biostability is also a relative term, meaning that a biostable linking moiety takes longer to break or requires certain cleavage conditions which hare less frequently encountered by the conjugate when present in physiological conditions.

According to some embodiments of the present invention, the linking moiety is a cleavable linker, or a biocleavable-linking moiety. In the context of some embodiments of the present invention, the linking moiety is a cleavable linker, or biocleavable linking moiety, which is selected so as to break and release the bioactive agent attached thereto at certain conditions, referred to herein as “drug-releasing conditions” or “cleavage conditions”. As used herein, the terms “biocleavable” and “biodegradable” are used interchangeably to refer to moieties that degrade (i.e., break and/or lose at least some of their covalent structure) under physiological or endosomal conditions. Biodegradable moieties are not necessarily hydrolytically degradable and may require enzymatic action to degrade.

As used herein, the terms “cleavable linker”, “biocleavable moiety” or “biodegradable moiety” describe a chemical moiety, which undergoes cleavage in a biological system such as, for example, the digestive system of an organism or a metabolic system in a living cell.

According to some embodiments of the present invention, the linking moiety is a photocleavable linker that cleaves upon light irradiation.

In some embodiments, the cleavable linker is selected according to its susceptibility to certain enzymes that are likely to be present at the targeted bodily site or at any other bodily site where cleavage is intended, thereby defining the cleavage conditions.

Representative examples of biocleavable moieties include, without limitation, amides, carboxylates, carbamates, phosphates, hydrazides, thiohydrazides, disulfides, epoxides, peroxo and methyleneamines. Such moieties are typically subjected to enzymatic cleavages in a biological system, by enzymes such as, for example, hydrolases, amidases, kinases, peptidases, phospholipases, lipases, proteases, esterases, epoxide hydrolases, nitrilases, glycosidases and the like.

For example, hydrolases (EC number beginning with 3) catalyze hydrolysis of a chemical bond according to the general reaction scheme A-B+H₂O→A-OH+B-H. Ester bonds are cleaved by sub-group of hydrolases known as esterases (EC number beginning with 3.1), which include nucleases, phosphodiesterases, lipases and phosphatases. Hydrolases having an EC number beginning with 3.4 are peptidases, which act on peptide bonds.

Additional information pertaining to enzymes, enzymatic reactions, and enzyme-linking moiety correlations can be found in various publically accessible sources, such as Bairoch A., “The ENZYME database in 2000”, Nucleic Acids Res, 2000, 28, pp. 304-305.

In some embodiments, the cleavable linker is selected to be more labile. By “more labile”, it is meant that some of the linking moieties have a higher tendency to break at given cleavage conditions compared to other linking moieties. In some embodiments wherein more than one cleavable linker is used in the conjugate, the linking moieties are selected according to a certain lability hierarchy that allows the design of a particular drug-releasing profile, and/or a particular multi-drug-releasing profile, wherein the order and the rate of drug release is controllable according to the lability hierarchy. In the context of some embodiment of the invention, the more labile linking moieties, higher in the lability hierarchy will break first and at a higher rate than those lower in the lability hierarchy. The ability to select linking moieties according to their lability hierarchy provides conjugates with differential multi-drug releasing profiles, according to some embodiments of the present invention.

The selection of the linking moieties according to lability hierarchy is determined according to the cleavage conditions, which the conjugate is expected to experience once it is administered into a living cell/tissue/organ (collectively referred to herein as a “bodily site”). Cleavage conditions include the chemical and physical conditions that are present in the bodily site, such as temperature, pH, the presence of reactive species and the presence of enzymes, all of which can cause a given linking moiety to break and release the bioactive agent attached thereto.

For example, some linking moieties are more labile (susceptible to) in higher temperatures, while others are susceptible to higher or lower pH values compared to other linking moieties. In such cases, a conjugate which is design to target a bodily site that is characterized by a localized pH value compared to its surroundings, an acid-labile or an H⁺-labile linking moiety is advantageously selected to release the bioactive agent it bears.

Bioactive Agent:

As discussed hereinabove, the conjugate is designed to carry a releasable payload, which can comprise a single bioactive agent, several copies of the same bioactive agent, linked by similar or different linking moieties, to control the release profile of the payload, or comprise of a series of different bioactive agents linked by similar or different linking moieties. In cases where the bioactive agents are the same, the conjugates of the present invention provide for substantial enhancement of the functionality of the bioactive agents, both in terms of localized release, concerted release or prolonged sequential release thereof. In cases where the bioactive agents are different one from one-another, the conjugates of the present invention provides for simultaneous, concerted or sequential release of the bioactive agents and can therefore be specifically advantageous in cases where the different bioactive agents confer a cumulative and/or a synergistic effect.

In the context of the present embodiments, the terms “bioactive agent”, and “pharmaceutically active agent” are used interchangeably. In some embodiments the bioactive agent is a drug.

As used herein, the terms “bioactive agent” and “drug” refer to small molecules or biomolecules that alter, inhibit, activate, or otherwise affect a biological mechanism or event. Bioactive agent that can be tethered to the conjugate, according to embodiments of the present invention, include, but are not limited to, anti-cancer substances for all types and stages of cancer and cancer treatments (chemotherapeutic, proliferative, acute, genetic, spontaneous etc.), anti-proliferative agents, photosensitizing agents, chemosensitizing agents, anti-inflammatory agents (including steroidal and non-steroidal anti-inflammatory agents and anti-pyretic agents), antimicrobial agents (including antibiotics, antiviral, antifungal, anti-parasite, anti-protozoan etc.), anti-oxidants, hormones, anti-hypertensive agents, anti-AIDS substances, anti-diabetic substances, immunosuppressants, enzyme inhibitors, neurotoxins, opioids, hypnotics, anti-histamines, lubricants, tranquilizers, anti-convulsants, muscle relaxants and anti-Parkinson substances, antipruritic agents, anti-spasmodics and muscle contractants including channel blockers, miotics and anti-cholinergics, anti-glaucoma compounds, modulators of cell-extracellular matrix interactions including cell growth inhibitors and anti-adhesion molecules, vitamins, vasodilating agents, inhibitors of DNA, RNA or protein synthesis, analgesics, anti-angiogenic factors, anti-secretory factors, anticoagulants and/or anti-thrombotic agents, anesthetics, ophthalmics, prostaglandins, anti-depressants, anti-psychotic substances, anti-emetics, radioactive agents and imaging agents. A more comprehensive listing of exemplary drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

As used herein, the term “small molecule” refers to molecules, whether naturally-occurring or artificially created (e.g., via chemical synthesis), that have a relatively low molecular weight. Typically, small molecules are monomeric and have a molecular weight of less than about 1500 Da. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, are all considered acceptable for use in accordance with the present invention.

Anti-cancer drugs that can be linked and controllably released from the conjugate according to some embodiments of the invention include, but are not limited to Chlorambucil; 3-(9-Acridinylamino)-5-(hydroxymethyl)aniline; Azatoxin; Acivicin; Aclarubicin; Acodazole

Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium;

Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Non-limiting examples of chemotherapeutic agents that can be efficiently delivered by the conjugates of the present invention, include amino containing chemotherapeutic agents such as camptothecin, daunorubicin, doxorubicin, N-(5,5-diacetoxypentyl)doxorubicin, anthracycline, mitomycin C, mitomycin A, 9-amino aminopertin, antinomycin, N⁸-acetyl spermidine, 1-(2-chloroethyl)-1,2-dimethanesulfonyl hydrazine, bleomycin, tallysomucin, and derivatives thereof; hydroxy containing chemotherapeutic agents such as etoposide, irinotecan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine and vinblastine, and derivatives thereof, sulfhydril containing chemotherapeutic agents and carboxyl containing chemotherapeutic agents. Additional chemotherapeutic agents include, without limitation, an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, a gonadotropin-releasing hormone analog, bleomycin, doxorubicin, paclitaxel, 4-OH cyclophosphamide and cisplatinum.

Anti-inflammatory drugs that can be linked and controllably released from the conjugate according to some embodiments of the invention include, but are not limited to Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

Suitable antimicrobial agents, including antibacterial, antifungal, antiprotozoal and antiviral agents, for use in context of the present invention include, without limitation, beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, farnesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole and mixtures thereof.

Non-limiting examples of anti-oxidants that are usable in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

Non-limiting examples of antihistamines usable in context of the present invention include chlorpheniramine, brompheniramine, dexchlorpheniramine, tripolidine, clemastine, diphenhydramine, promethazine, piperazines, piperidines, astemizole, loratadine and terfenadine.

Representative examples of hormones include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone and pharmaceutically acceptable esters and salts thereof, and combinations of any of the foregoing.

Non-limiting examples of analgesic agents that can be efficiently delivered by the conjugates of the present invention, include acetaminophen, alfentanil hydrochloride, aminobenzoate potassium, aminobenzoate sodium, anidoxime, anileridine, anileridine hydrochloride, anilopam hydrochloride, anirolac, antipyrine, aspirin, benoxaprofen, benzydamine hydrochloride, bicifadine hydrochloride, brifentanil hydrochloride, bromadoline maleate, bromfenac sodium, buprenorphine hydrochloride, butacetin, butixirate, butorphanol, butorphanol tartrate, carbamazepine, carbaspirin calcium, carbiphene hydrochloride, carfentanil citrate, ciprefadol succinate, ciramadol, ciramadol hydrochloride, clonixeril, clonixin, codeine, codeine phosphate, codeine sulfate, conorphone hydrochloride, cyclazocine, dexoxadrol hydrochloride, dexpemedolac, dezocine, diflunisal, dihydrocodeine bitartrate, dimefadane, dipyrone, doxpicomine hydrochloride, drinidene, enadoline hydrochloride, epirizole, ergotamine tartrate, ethoxazene hydrochloride, etofenamate, eugenol, fenoprofen, fenoprofen calcium, fentanyl citrate, floctafenine, flufenisal, flunixin, flunixin meglumine, flupirtine maleate, fluproquazone, fluradoline hydrochloride, flurbiprofen, hydromorphone hydrochloride, ibufenac, indoprofen, ketazocine, ketorfanol, ketorolac tromethamine, letimide hydrochloride, levomethadyl acetate, levomethadyl acetate hydrochloride, levonantradol hydrochloride, levorphanol tartrate, lofemizole hydrochloride, lofentanil oxalate, lorcinadol, lornoxicam, magnesium salicylate, mefenamic acid, menabitan hydrochloride, meperidine hydrochloride, meptazinol hydrochloride, methadone hydrochloride, methadyl acetate, methopholine, methotrimeprazine, metkephamid acetate, mimbane hydrochloride, mirfentanil hydrochloride, molinazone, morphine sulfate, moxazocine, nabitan hydrochloride, nalbuphine hydrochloride, nalmexone hydrochloride, namoxyrate, nantradol hydrochloride, naproxen, naproxen sodium, naproxol, nefopam hydrochloride, nexeridine hydrochloride, noracymethadol hydrochloride, ocfentanil hydrochloride, octazamide, olvanil, oxetorone fumarate, oxycodone, oxycodone hydrochloride, oxycodone terephthalate, oxymorphone hydrochloride, pemedolac, pentamorphone, pentazocine, pentazocine hydrochloride, pentazocine lactate, phenazopyridine hydrochloride, phenyramidol hydrochloride, picenadol hydrochloride, pinadoline, pirfenidone, piroxicam olamine, pravadoline maleate, prodilidine hydrochloride, profadol hydrochloride, propiram fumarate, propoxyphene hydrochloride, propoxyphene napsylate, proxazole, proxazole citrate, proxorphan tartrate, pyrroliphene hydrochloride, remifentanil hydrochloride, salcolex, salethamide maleate, salicylamide, salicylate meglumine, salsalate, sodium salicylate, spiradoline mesylate, sufentanil, sufentanil citrate, talmetacin, talniflumate, talosalate, tazadolene succinate, tebufelone, tetrydamine, tifurac sodium, tilidine hydrochloride, tiopinac, tonazocine mesylate, tramadol hydrochloride, trefentanil hydrochloride, trolamine, veradoline hydrochloride, verilopam hydrochloride, volazocine, xorphanol mesylate, xylazine hydrochloride, zenazocine mesylate, zomepirac sodium and zucapsaicin.

Non-limiting examples of photosensitizers include photofrin, photoporphyrin, benzoporphyrin, tookad, antrin, purlytin, foscan, and halogenated dyes disclosed, e.g. in U.S. Pat. Nos. 9,572,881, 9,040,721, 8,962,797, 8,748,446 and EP2850061.

Targeting Moiety:

As used herein, the term “targeting moiety” describes a molecular entity that exhibits an affinity to a desired bodily site (e.g., particular organ, cells and/or tissues). In some embodiments, a targeting moiety is specific to certain targets. The target is typically a biomolecule that occurs at a higher concentration or exclusively at the targeted bodily site. In some embodiments, the targeting moiety is a biomolecule or a derivative thereof that has a specific and relatively high affinity to the target.

Targeting moieties are often employed as the bimolecular carrier in order to direct a drug to specific structures in the body or sites of physiological functions. According to some embodiments, a targeting moiety is a compound with structure or site specific reactivity.

Exemplary targeting agents include, without limitation, peptides, proteins, porphyrins, hormones, antigens, haptens, antibodies and fragments thereof, DNA fragments, RNA fragments and analogs and derivatives thereof, and any receptor ligands that bind to receptors that are expressed specifically or more abundantly at the targeted bodily sites.

As used herein, the term “biomolecule” refers to molecules (e.g., polypeptides, amino acids, polynucleotides, nucleotides, polysaccharides, sugars, lipids, nucleoproteins, glycoproteins, lipoproteins, steroids, metabolites, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

In some embodiments, a targeting moiety comprises a cell-internalizing moiety, such that the molecular structure can more readily penetrate a targeted cell. Exemplary cell-internalizing moieties include, without limitation, positively charges (at physiological environment) moieties such as guanidines and amines, and moieties containing same (e.g., arginine and lysine).

In some embodiments, the targeting moiety exhibits a specific affinity to cancerous cells and neoplastic tissues. Such targeting moieties may be used to target the molecular structure presented herein, thereby delivering anticancerous bioactive agents, according to some embodiments of the present invention, to cancerous cells and tissues. The result is an enhanced effect and an improved exposure of the cancerous cells and neoplastic tissues to the anticancerous bioactive agent, preferably accompanied by reduced exposure of non-cancerous cells to the anticancerous bioactive agents.

A class of compounds that is suitable as targeting moieties, according to some embodiments of the present invention, are short peptides and peptide analogs, generally referred to herein as peptidomimetic compounds, that display more favorable pharmacological properties than their prototype native peptides. The native peptide itself, the pharmacological properties of which have been optimized, generally serves as a lead for the development of these peptidomimetics. In general, a small number of amino acids (usually four to eight) are responsible for the biological activity (recognition and binding; targeting) of a peptide ligand (targeting moiety) by a receptor (target). Once this biologically active site is determined, a lead structure for development of peptidomimetic can be optimized, for example by molecular modeling programs. U.S. Pat. Nos. 5,811,392, 6,407,059 and 7,084,244, which are incorporated herein by reference in their entirety, describe the preparation and use of a class of cyclic peptidomimetic targeting moieties, which can be used in the context of some embodiments of the present invention.

Peptide nucleic acid (PNA) constitute an exemplary class of targeting moiety that may be used in the context of some embodiments of the present invention. U.S. Pat. No. 6,395,474, which is incorporated herein by reference in its entirety, describes PNA as an analogue of DNA in which the phosphodiester backbone of DNA is replaced with a pseudo-peptide such as N-(2-amino-ethyl)-glycine. Methylenecarbonyl linkers attach DNA, RNA, or synthetic nucleobases to the polyamide backbone. PNA, obeying Watson-Crick hydrogen bonding rules, mimics the behavior of DNA and RNA by binding to complementary nucleic acid sequences such as those found in DNA, RNA, and other PNAs. An exemplary molecular structure utilizing PNA, according to some embodiments of the present invention, may bind, for example, to a specific mutated nucleic acid sequence found in the DNA of a cancerous tumor.

One example of a class of targeting moieties, which can be used advantageously in the context of embodiments of the present invention, is the family of tumor-targeting moieties that bind selectively to αᵥβ₃ and αᵥβ₅ integrins, referred to herein as the RGD (Arg-Gly-Asp) family [Arap, W. et al., Science, 1998, 279(5349):377-80]. Short peptides and peptidomimetic analogs, which are based on the RGD motif and exhibit is biological binding activity, can be used as targeting moieties in a molecular structure, according to some embodiments of the present invention, to inhibit the growth and possibly eradicate tumors in the treatment of cancer.

Additional targeting moieties, which can be used effectively in the context of the molecular structures presented herein for treating cancer, are described in the literature [e.g., “Novel Oncology Therapeutics: Targeted Drug Delivery for Cancer”, Journal of Drug Delivery, Vol. 2013, 2013].

Non-limiting examples of targeting moieties which are useful in the context of some embodiments of the present invention include octreotide (OCT), lanreotide, pasireotide, vapreotide, cilengitide analog c(RGDfK), and luteinizing Hormone-Releasing Hormone (LHRH), bombesin, and arginine-glycine-aspartic acid (RGD).

Uses and Applications:

The conjugates presented herein can be used to quantitatively monitor the release of a bioactive agent in the targeted bodily site or tissue. In the case of a dual-fluorophore conjugate, the equation can be used:

R_{eff.}=n_{Swi-on}/n_{ref.} ˜I_{Swi signal}/I_{Ref signal} (see, FIG. 3), where n_{Swi-on} and n_{ref.} are the number of switchable fluorophores in the “on” form and the number of reference fluorophores, respectively, and I_{Swi signal} and I_{Ref signal} are their luminescence signals.

The equation for both a single and a double fluorophore conjugate can also be written as:

R_{eff.}˜I_{Swi signal}/I_{Ref signal}, where I_{Swi signal} and I_{Ref signal} are the two luminescence signal values (two luminescence intensities; or contributions in two luminescence lifetimes).

Or:

R_{eff}=I_{Swi signal}/I_{Ref signal}), where k is a calibration coefficient.

Calibration may be required because the transparence (absorption) light in body is dependent on the wavelength and the luminescence lifetime might be affected by environment. Calibration coefficient k can be found experimentally or calculated theoretically.

The conjugate presented herein can be used to provide personal medical treatment and diagnostics of a subject in need of a bioactive agent for the treatment of a medical condition treatable with the bioactive agent. While the mode of administration of the conjugate, and determination of efficacy of the bioactive agent remain similar to those used in the case of other known drug delivery conjugates, the presently provided conjugates offer the quantitative determination of the rate of release of the bioactive agent in the targeted cells of the subject. The quantitative determination of the actual delivered bioactive agent allows for managing the treatment in a more controlled and personalized manner, rather than basing the regimen of treatment on crude estimation of the amount of bioactive agent.

Quantitative determination of the amount of delivered bioactive agent, together with the determination of efficacy of the bioactive agent in the subject, allows the caretaker to fine-tune the regimen in order to optimize the balance between beneficial and adverse side effects of bioactive agent.

Methods and conditions of storage, preparation (including the concentrations and solvent systems), administration and monitoring of the ratiometric theranostic conjugates are the same as for the conventional, single-signal theranostic conjugates. The concentration of conjugates in tissue that allows luminescence monitoring is in general 0.1-50 μM. Specificity of the use of the ratiometric theranostic conjugates as compared to the conventional theranostic conjugates is the need to measure two luminescence signals and then to take the ratio between the switchable and the reference signals. Both the switchable and the reference signals can independently increase or decrease or the reference signal can remain constant.

Medical Conditions:

The conjugate presented herein can be used to treat any medical condition that is treatable by administration of a bioactive agent (drug). According to some embodiments of the present invention, it is advantageous to use the conjugate to treat medical conditions, which are treatable by administration of a combination of drugs. In some embodiments, the medical condition includes an autoimmune disease, a genetic disease, a degenerative disease, a psychiatric or mental disease or condition. In some embodiments, the medical condition includes a peptic ulcer disease, Alzheimer's disease, rheumatoid arthritis, post-traumatic stress disorder, Crohn's disease, tuberculosis, leprosy, malaria and HIV/AIDS.

According to some embodiments, the degenerative disease includes Alzheimer's disease, Amyotrophic Lateral Sclerosis (ALS), a.k.a., Lou Gehrig's Disease, Osteoarthritis, Atherosclerosis, Cancer, Charcot Marie Tooth Disease (CMT), Chronic Obstructive Pulmonary Disease (COPD), Chronic traumatic encephalopathy, Diabetes, Ehlers-Danlos Syndrome, Essential tremor, Friedreich's ataxia, Leg Disease, Huntington's Disease, Inflammatory Bowel Disease (IBD), Keratoconus, Keratoglobus, Macular degeneration, Marfan's Syndrome, Multiple sclerosis, Multiple system atrophy, Muscular dystrophy, Niemann Pick disease, Osteoporosis, Parkinson's Disease, Progressive supranuclear palsy, Prostatitis, Retinitis Pigmentosa, Rheumatoid Arthritis, and Tay-Sachs Disease.

According to some embodiments, the autoimmune disease includes Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo and Wegener's granulomatosis (now termed Granulomatosis with Polyangiitis (GPA).

In some embodiments of the present invention, the medical condition is associated with an infection caused by a pathogenic microorganism, including a viral infection, a bacterial infection, a yeast infection, a fungal infection, a protozoan infection, a parasite-related infection and the like.

Medical conditions associated with a pathogenic microorganism include, without limitation, actinomycosis, anthrax, aspergillosis, bacteremia, bacterial, bacterial skin diseases, bartonella infections, botulism, brucellosis, burkholderia infections, campylobacter infections, candidiasis, cat-scratch disease, chlamydia infections, cholera, clostridium infections, coccidioidomycosis, cryptococcosis, dermatomycoses, dermatomycoses, diphtheria, ehrlichiosis, epidemic louse borne typhus, Escherichia coli infections, fusobacterium infections, gangrene, general infections, general mycoses, gram-negative bacterial infections, Gram-positive bacterial infections, histoplasmosis, impetigo, klebsiella infections, legionellosis, leprosy, leptospirosis, listeria infections, lyme disease, maduromycosis, melioidosis, mycobacterium infections, mycoplasma infections, necrotizing fasciitis, nocardia infections, onychomycosis, ornithosis, pneumococcal infections, pneumonia, pseudomonas infections, Q fever, rat-bite fever, relapsing fever, rheumatic fever, rickettsia infections, Rocky-mountain spotted fever, salmonella infections, scarlet fever, scrub typhus, sepsis, sexually transmitted bacterial diseases, staphylococcal infections, streptococcal infections, surgical site infection, tetanus, tick-borne diseases, tuberculosis, tularemia, typhoid fever, urinary tract infection, vibrio infections, yaws, yersinia infections, Yersinia pestis plague, zoonoses and zygomycosis.

Non-limiting examples of pathogenic fungi include genus Absidia: Absidia corymbifera; genus Ajellomyces: Ajellomyces capsulatus, Ajellomyces dermatitidis; genus Arthroderma: Arthroderma benhamiae, Arthroderma fulvum, Arthroderma gypseum, Arthroderma incurvatum, Arthroderma otae, Arthroderma vanbreuseghemii; genus Aspergillus: Aspergillus flavus, Aspergillus fumigatus, Aspergillus niger; genus Blastomyces: Blastomyces dermatitidis; genus Candida: Candida albicans, Candida glabrata, Candida guilliermondii, Candida krusei, Candida parapsilosis, Candida tropicalis, Candida pelliculosa; genus Cladophialophora: Cladophialophora carrionii; genus Coccidioides: Coccidioides immitis; genus Cryptococcus: Cryptococcus neoformans; genus Cunninghamella: Cunninghamella sp.; genus Epidermophyton: Epidermophyton floccosum; genus Exophiala: Exophiala dermatitidis; genus Filobasidiella: Filobasidiella neoformans; genus Fonsecaea: Fonsecaea pedrosoi; genus Fusarium: Fusarium solani; genus Geotrichum: Geotrichum candidum; genus Histoplasma: Histoplasma capsulatum; genus Hortaea: Hortaea werneckii; genus Issatschenkia: Issatschenkia orientalis; genus Madurella: Madurella grisae; genus Malassezia: Malassezia furfur, Malassezia globosa, Malassezia obtusa, Malassezia pachydermatis, Malassezia restricta, Malassezia slooffiae, Malassezia sympodialis; genus Microsporum: Microsporum canis, Microsporum fulvum, Microsporum gypseum; genus Mucor: Mucor circinelloides; genus Nectria: Nectria haematococca; genus Paecilomyces: Paecilomyces variotii; genus Paracoccidioides: Paracoccidioides brasiliensis; genus Penicillium: Penicillium marneffei; genus Pichia, Pichia anomala, Pichia guilliermondii; genus Pneumocystis: Pneumocystis carinii; genus Pseudallescheria: Pseudallescheria boydii; genus Rhizopus: Rhizopus oryzae; genus Rhodotorula: Rhodotorula rubra; genus Scedosporium: Scedosporium apiospermum; genus Schizophyllum: Schizophyllum commune; genus Sporothrix: Sporothrix schenckii; genus Trichophyton: Trichophyton mentagrophytes, Trichophyton rubrum, Trichophyton verrucosum, Trichophyton violaceum; and genus Trichosporon: Trichosporon asahii, Trichosporon cutaneum, Trichosporon inkin, Trichosporon mucoides.

Non-limiting examples of other pathogenic microorganism include Acanthamoeba and other free-living amoebae, Aeromonas hydrophila, Anisakis and related worms, Ascaris lumbricoides, Bacillus cereus, Campylobacter jejuni, Clostridium botulinum, Clostridium perfringens, Cryptosporidium parvum, Cyclospora cayetanensis, Diphyllobothrium, Entamoeba histolytica, Eustrongylides, Giardia lamblia, Listeria monocytogenes, Nanophyetus, Plesiomonas shigelloides, Salmonella, Shigella, Staphylococcus aureus, Streptococcus, Trichuris trichiura, Vibrio cholerae, Vibrio parahaemolyticus, Vibrio vulnificus and other vibrios, Yersinia enterocolitica and Yersinia pseudotuberculosis.

Cancer Treatment and Chemotherapy:

In some embodiments of the present invention, the medical condition is associated with malignant cells and tumors, collectively referred to herein as cancer.

To date, chemotherapy remains the most common and most frequently used in cancer treatment, alone or in combination with other therapies. Currently available anticancer chemotherapies act by affecting specific molecular targets in proliferating cancer cells, leading to inhibition of essential intracellular processes such as DNA transcription, synthesis and replication.

Unfortunately anticancerous drugs are highly toxic, as they are designed to kill mammalian cells, and are therefore harmful also to normal proliferating cells resulting in debilitating and even lethal side effects. Some of these adverse effects are gastrointestinal toxicity, nausea, vomiting, and diarrhea when the epithelial lining of the intestine is affected. Other side effects include alopecia, when the hair follicles are attacked, bone marrow suppression and neutropenia due to toxicity of hematopoietic precursors. Therefore the effectiveness of currently used anticancerous drugs is dose-limited due to their toxicity to normal rapidly growing cells. The use of a conjugate according to embodiments of the present invention, can optimize the balance between the desired anticancer activity of certain anticancer drugs and their adverse side effects, by quantitative determination of the actual amount of drug released in the targeted cells.

One of the contemporary approaches in the fight against cancer is engineering of molecular targeted drugs that permeate cancer cells and specifically modulate activity of molecules that belong to signal-transduction pathways. These targets include products of frequently mutated oncogenes, such as k-Ras and other proteins that belong to tyrosine kinase signal transduction pathways. For example, Imatinib (Gleevec®), is the first such drug, approved for treatment of chronic myelogenous leukemia (CML). Imatinib blocks the activity of non-receptor tyrosine kinase BCR-Abl oncogene, present in 95% of patients with CML. Imatinib was found to be effective in the treatment of CML and certain tumors of the digestive tract. Nevertheless, as others, this new compound is not completely specific to its target; therefore side effects emerge, including severe congestive cardiac failure, pulmonary tuberculosis, liver toxicity, sweet syndrome (acute febrile neutrophilic dermatosis), leukocytosis, dermal edemas, nausea, rash and musculoskeletal pain.

Angiogenesis inhibitors are currently investigated for their use in cancer treatment and to date, one anti-angiogenetic drug, Bevacizumab (Avastin®), was approved for the treatment of solid tumors in combination with standard chemotherapy. However, as in all chemotherapeutic drugs, Bevacizumab causes a number of adverse side effects such as hypertension, blood clots, neutropenia, neuropathy, proteinuria and bowel perforation.

In some embodiments, the targeting moiety of the conjugates presented herein, is responsible for the higher concentration of the conjugate at the targeted bodily site compared to non-targeted bodily sites, thereby reducing the adverse side effects associated with the toxicity of the anti-cancer drugs attached thereto. In addition, the linking moieties attached the anti-cancer drugs to the conjugate are selected such that they cleave in conditions that are present at the targeted site more so than in non-targeted sites, thereby releasing the payload of drugs at the targeted site at a higher rate compared to non-targeted sites.

In the context of some embodiments of the present invention, the term “cancer” refers, but not limited to acute lymphoblastic, acute lymphoblastic leukemia, acute lymphocytic leukemia, acute myelogenous leukemia, acute myeloid leukemia, adrenocortical carcinoma, AIDS-related lymphoma, anal cancer, appendix cancer, basal-cell carcinoma, bladder cancer, brain cancer, brainstem glioma, breast cancer, bronchial adenomas/carcinoids, Burkitt's lymphoma, carcinoid tumor, cerebellar or cerebral astrocytoma, cervical cancer, cholangiocarcinoma, chondrosarcoma, chronic lymphocytic or chronic lymphocytic leukemia, chronic myelogenous or chronic myeloid leukemia, chronic myeloproliferative disorders, colon cancer, cutaneous T-cell lymphoma, desmoplastic small round cell tumor, endometrial uterine cancer, ependymoma, esophageal cancer, Ewing's sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal tumor (GIST), gestational trophoblastic tumor, glioma of the brain stem, hairy cell leukemia, head and neck cancer, heart cancer, hepatocellular (liver) cancer, Hodgkin lymphoma, hypopharyngeal cancer, hypothalamic and visual pathway glioma, intraocular melanoma, Islet cell carcinoma, Kaposi sarcoma, laryngeal cancer, leukaemia, lip and oral cavity cancer, liposarcoma, lymphoma, male breast cancer, malignant mesothelioma, medulloblastoma, melanoma, Merkel cell skin carcinoma, mesothelioma, metastatic squamous neck cancer, mouth cancer, multiple endocrine neoplasia syndrome, multiple myeloma, multiple myeloma/plasma cell neoplasm, mycosis fungoides, myelodysplastic/myeloproliferative diseases, nasal cavity and paranasal sinus cancer, nasopharyngeal carcinoma, neuroblastoma, non-Hodgkin lymphoma, non-melanoma skin cancer, non-small cell lung cancer, oligodendroglioma, oral cancer, oropharyngeal cancer, osteosarcoma and malignant fibrous histiocytoma, ovarian cancer, ovarian germ cell tumor, ovarian epithelial cancer (surface epithelial-stromal tumor), ovarian low malignant potential tumor, pancreatic cancer, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia, pleuropulmonary blastoma, primary carcinoma, primary central nervous system lymphoma, primary liver cancer, prostate cancer, rectal cancer, renal cell carcinoma, renal pelvis and ureter carcinoma, retinoblastoma, rhabdomyosarcoma, salivary gland cancer, Sézary syndrome, small cell lung cancer, small intestine cancer, soft tissue sarcoma, squamous cell carcinoma, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine sarcoma, vaginal cancer, visual pathway and hypothalamic glioma, vulvar cancer, Waldenström macroglobulinemia and Wilms tumor.

It is expected that during the life of a patent maturing from this application many relevant ratiometric luminescent theranostic conjugates will be developed and the scope of the term ratiometric luminescent theranostic conjugates is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Drug Delivery System for Real Time Monitoring of Drug Release

As discussed hereinabove, targeted drug delivery (TDD) is an efficient strategy for cancer treatment; however, the real time monitoring of drug delivery is still challenging because of a pronounced lack of turn-on, near-infrared (NIR) fluorescent dyes (reporters) able for detecting drug release events in vitro and in vivo. The example presented below provides a TDD system OCTA-G-XCy-CLB, according to some embodiments of the present invention, comprising NIR switchable reporter based on xanthene-cyanine dye (XCy) attached to an anticancer drug chlorambucil (CLB) via a biodegradable ester linker, and coupled also to a targeting peptide octreotide amide (OCTA) specific to somatostatin receptors (SSTR) on tumor cells. This OCTA-G-XCy-CLB conjugate exhibits no detectable fluorescence, while upon the environment-mediated cleavage of the linker, a bright fluorescence appears at about 710 nm, signaling the release of the CLB drug. The real time TDD monitoring has been demonstrated in the example of human pancreatic cancer (Panc-1) and Chinese hamster ovary (CHO) cell lines.

Scheme 1 presents the presently provided TDD system OCTA-G-XCy-CLB.

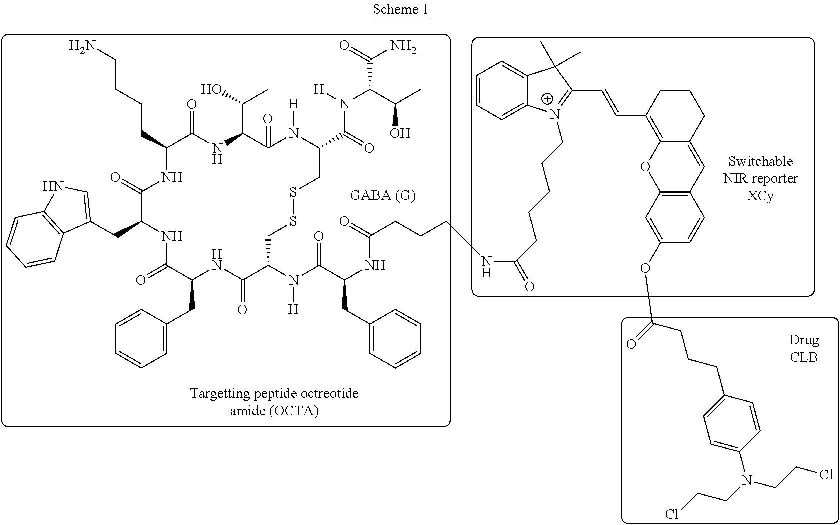 [SMILES: C[C@@H](O)[C@H](NC(=O)[C@@H]1CSSC[C@H](NC(=O)[C@H](Cc2ccccc2)NC(=O)CCCNC(=O)CCCCC[N+]2=C(/C=C/C3=C4Oc5cc(OC(=O)CCCc6ccc(N(CCCl)CCCl)cc6)ccc5C=C4CCC3)C(C)(C)c3ccccc32)C(=O)N[C@@H](Cc2ccccc2)C(=O)N[C@@H](Cc2c[nH]c3ccccc23)C(=O)N[C@@H](CCCCN)C(=O)N[C@@H]([C@@H](C)O)C(=O)N1)C(N)=O]

The synthesis and investigation of a TDD system, OCTA-G-XCy-CLB (see Scheme 1). CLB is a chemotherapy medication used to treat chronic lymphocytic leukemia. OCTA is an analog of cyclic, S—S bridged octa-peptide (sandostatin) which targets overexpressed somatostatin receptors (SSTR) on tumor cells. Amino acid GABA (G) was used as a linker moiety for connecting (OCTA) and XCy.

The dye XCy containing reactive carboxylic group for binding to targeting carrier was synthesized as presented. The synthetic strategy consisted of the straightforward nucleophilic substitution of chlorine atom in pre-synthesized dye Cy with resorcinol to eliminate the indolenine moiety by a retro-Knovenagel reaction followed by cyclization and dehydration to the aimed dye XCy (Scheme 2).

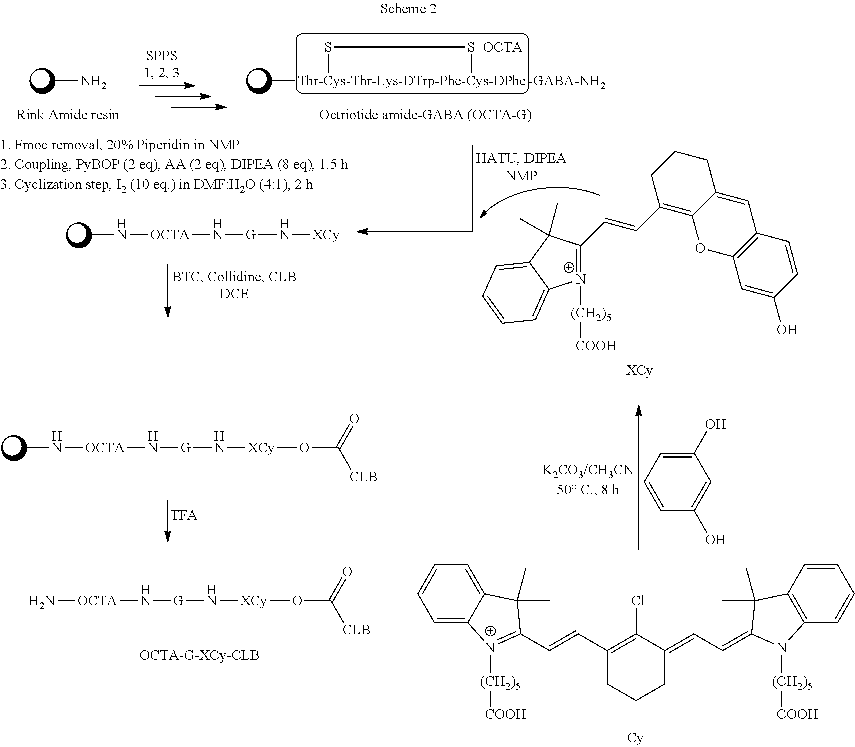

The TDD system OCTA-G-XCy-CLB was synthesized through solid phase peptide synthesis. The peptide sequences were built upon a solid support (rink amide resin) using Fmoc chemistry protocol. After the synthesis of the linear peptide, the N-terminal Fmoc group was removed and the peptide was cyclized by the treatment with excess 12 in DMF. The obtained targeting peptide OCTA immobilized on a rink amide resin was attached to XCy. Thereafter, the CLB carboxyl function was activated by treatment with BTC and collidine in DCE and coupled to the XCy hydroxyl group. Then, the obtained TDD system was removed from the resin by TFA.

To estimate the effect of OCTA on the spectral properties and drug release rate, the dye-drug conjugate XCy-CLB was also synthesized and investigated. The synthesis consisted in pre-activation of CLB with thionyl chloride in DCM followed by condensation of the obtained CLB carboxylic acid chloride with XCy hydroxyl group (see, Scheme 3).

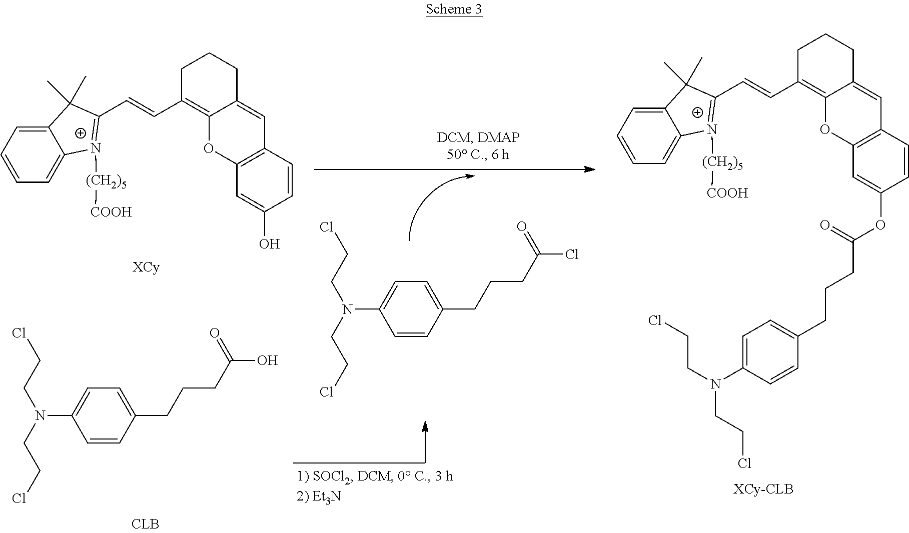

As a result, the drug and reporter were bound to each other by means of a biodegradable ester linker. The detailed synthetic procedures and characterization data for XCy, XCy-CLB and OCTA-G-XCy-CLB are presented herein below.

The absorption and emission spectra of XCy, XCy-CLB and OCTA-G-XCy-CLB were investigated in two different conditions, 10 mM phosphate buffer pH 7.4 containing 10% methanol (PB) and cell medium pH 7.4 containing 10% methanol (CM) (see, Table 1 below). CM was taken as a model of normal physiological conditions during drug delivery in the experiments with cells. A RPMI-1640 standard cell medium that contains 50 mL fetal bovine serum (FBS), 5 mL penicillin (10,000 U/mL), 5 mL streptomycin (10 mg/mL), 5 mL of 200 mM glutamine, and 5.3 mg/L phenol red indicator was utilized. FBS contains enzyme aspartate-aminotransferase that facilitates the ester bond cleavage.

TABLE 1

| | PB | | CM | |
|---|---|---|---|---|
| Reporter/ | λₘₐₓAbs | λₘₐₓFl | λₘₐₓAbs | λₘₐₓFl |
| Conjugate | (nm) | (nm) | (nm) | (nm) |
| XCy | 681 | 708 | 679 | 713 |
| XCy-CLB | 598 | No fl. | 602 | No fl. |
| OCTA-G-XCy- | 604 | No fl. | 604 | No fl. |
| CLB | | | | |

Free dye XCy (“on” form) in PB and CM has the absorption maximum about 680 nm and strong NIR fluorescence at around 710 nm, as can be seen in FIG. 4, line A and line B.

FIG. 4 presents a plot showing the spectral properties of XCy in PB (line A), XCy in CM (line B), XCy-CLB in PB (line C), XCy-CLB in CM (line D), OCTA-G-XCy-CLB in PB (line E), and OCTA-G-XCy-CLB in CM (line F), and (line A) and (line B)—XCy exists in the “on” form; (line C), (line D) and (line E)—XCy exists in the “off” form.

The absorption maximum of XCy bound to CLB (“off” form) in both conjugates XCy-CLB and OCTA-G-XCy-CLB is about 80 nm blue shifted to ˜600 nm (see, FIG. 4, lines C, D and E) and the fluorescence is not detectable. Therefore, hydrolytic cleavage of the ester bond in XCy-CLB and OCTA-G-XCy-CLB to release free CLB results in the dramatic increase of fluorescence, which can be used for monitoring the drug release.

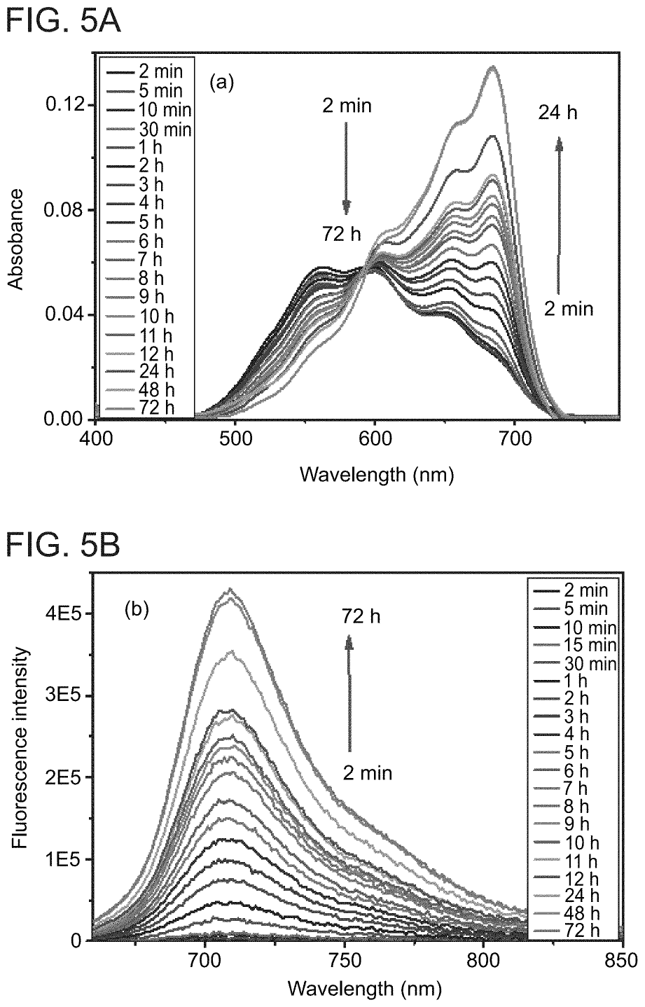

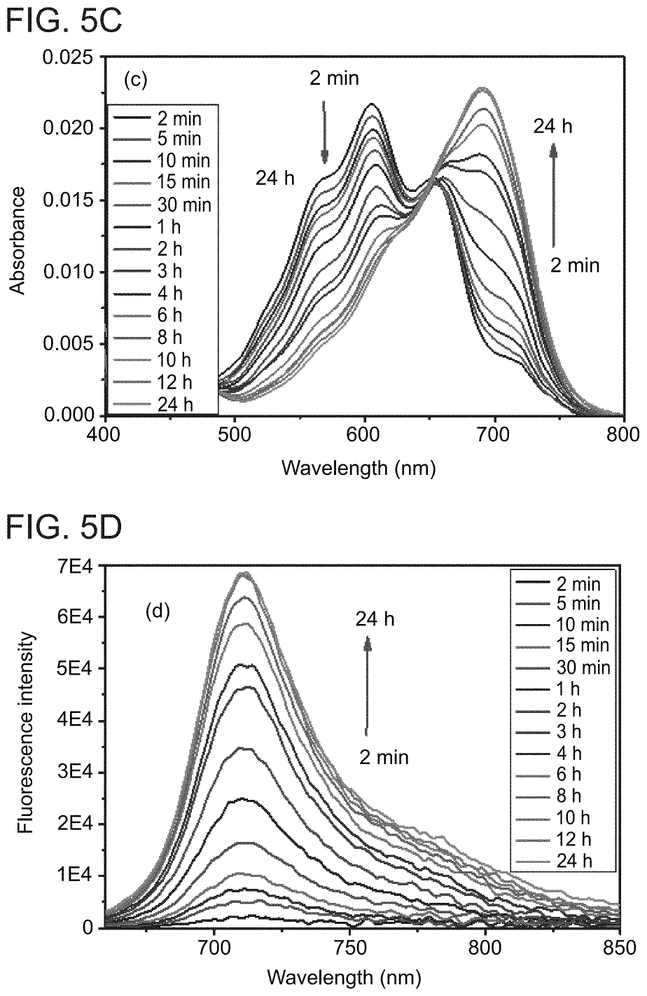

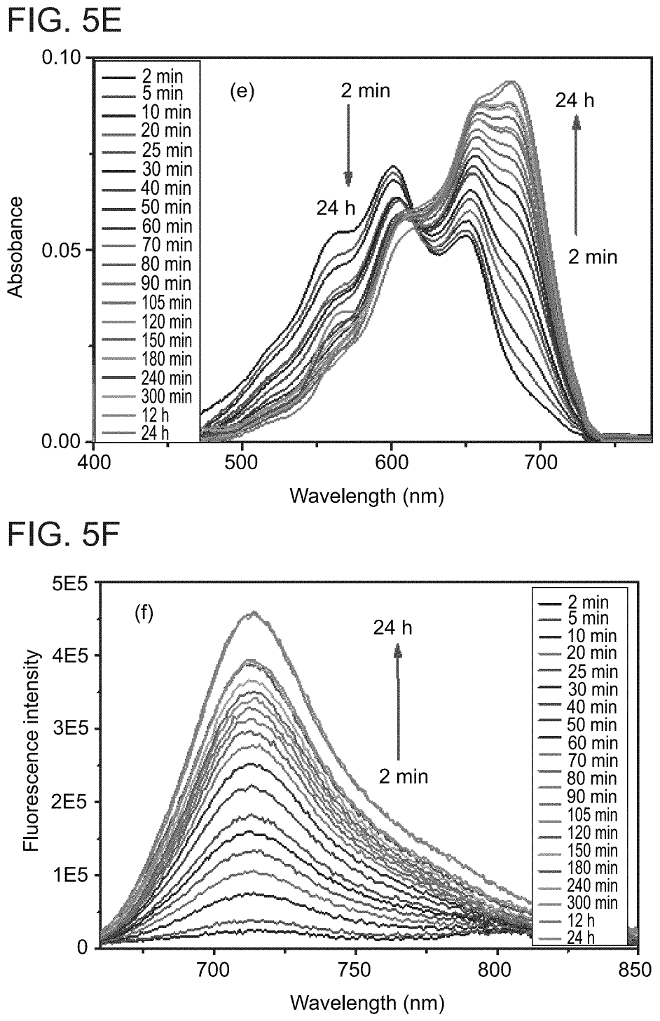

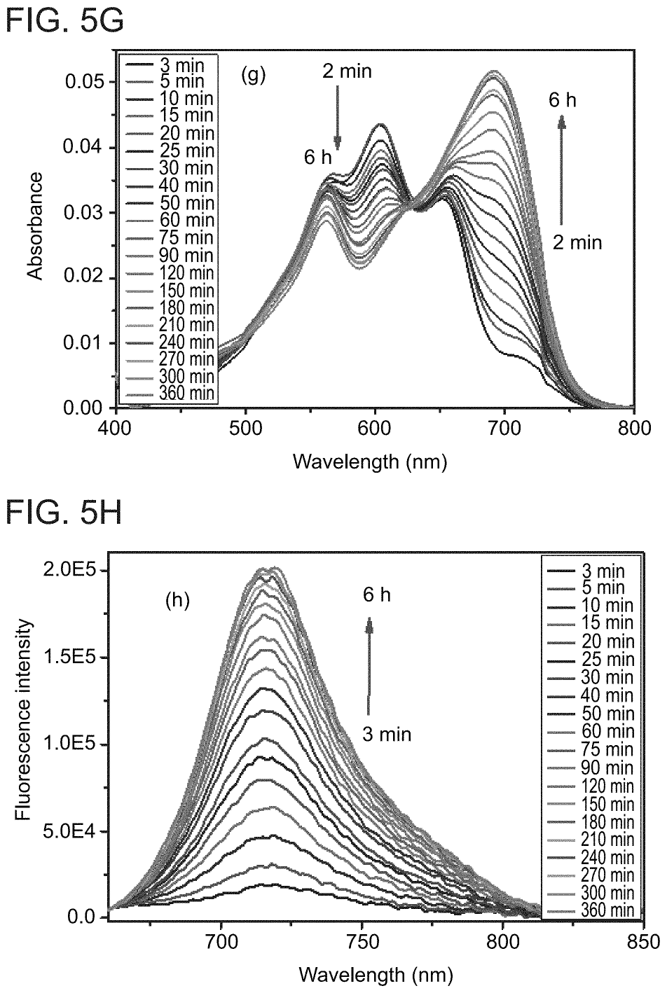

To estimate the rate of the CLB release, the time-dependent absorption and fluorescence spectra of XCy-CLB and OCTA-G-XCy-CLB were measured in PB and CM (see, FIGS. 5A-H). With respect to time, the absorption band at about 600 nm decreases, a new band at about 680 nm increases and a fluorescence band subsequently appears at ˜708 nm (λₑₓ=650 nm).

FIGS. 5A-H present time dependent absorption (FIGS. 5A, C, E and G) and fluorescence (FIGS. 5B, D, F and H) spectra of XCy-CLB (FIGS. 5A, B, C and D) and OCTA-G-XCy-CLB (FIGS. 5E, F, G and H) in PB (FIGS. 5A, B, E and F) and CM (FIGS. 5C, D, G and H), whereas λₑₓ=650 nm.

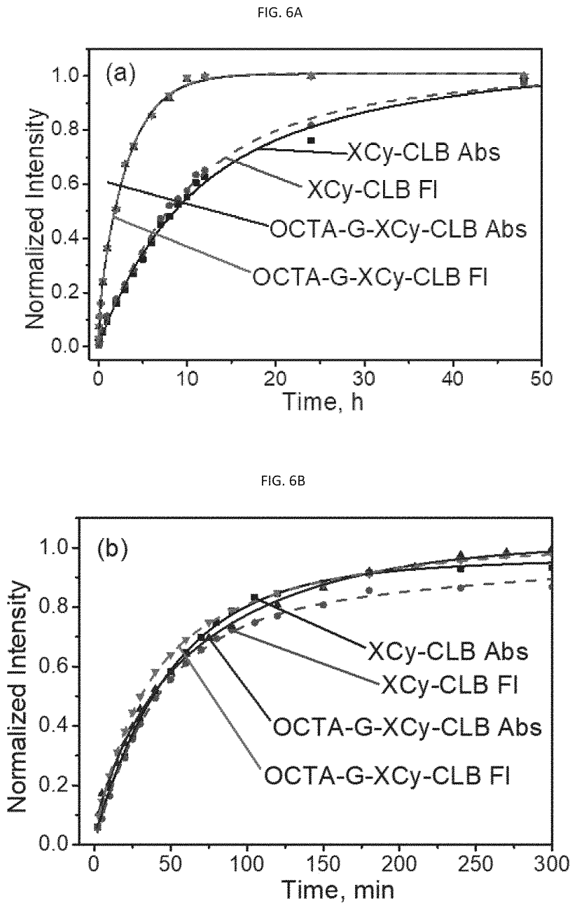

Based of the time-dependent absorption and emission spectra, corresponding drug cleavage profiles were obtained (see, FIGS. 6A-B) and the drug release rates were quantified by half-lives (τ_{1/2}). The cleavage profiles and half-lives obtained spectrophotometrically and spectrofluorometrically almost coincide. Due to the presence of esterase in CM, the CLB release from the XCy-CLB (see, FIGS. 6A-B) conjugate in this media (τ_{1/2}˜40 min) was found to be about 12-fold faster compared to that in PB (τ_{1/2}˜8 h). The release of CLB from OCTA-G-XCy-CLB in CM (τ_{1/2}˜25 min) is 4-fold faster than that in PB τ_{1/2} ˜2.5 h. Thus, the drug release for both XCy-CLB and OCTA-G-XCy-CLB in CM is much faster compared to PB. The obtained half-lives of the ester bond cleavage in these media were found in good agreement with some reported data. Linkage of OCTA to the conjugate accelerates the drug release by factor of 6 in PB and 1.3 in CM.

FIGS. 6A-B present spectrophotometrically (Abs) and spectrofluorometrically (Fl) estimated cleavage profiles for conjugate XCy-CLB and OCTA-G-XCy-CLB in PB pH 7.4 (FIG. 6A) and cell medium (FIG. 6B).

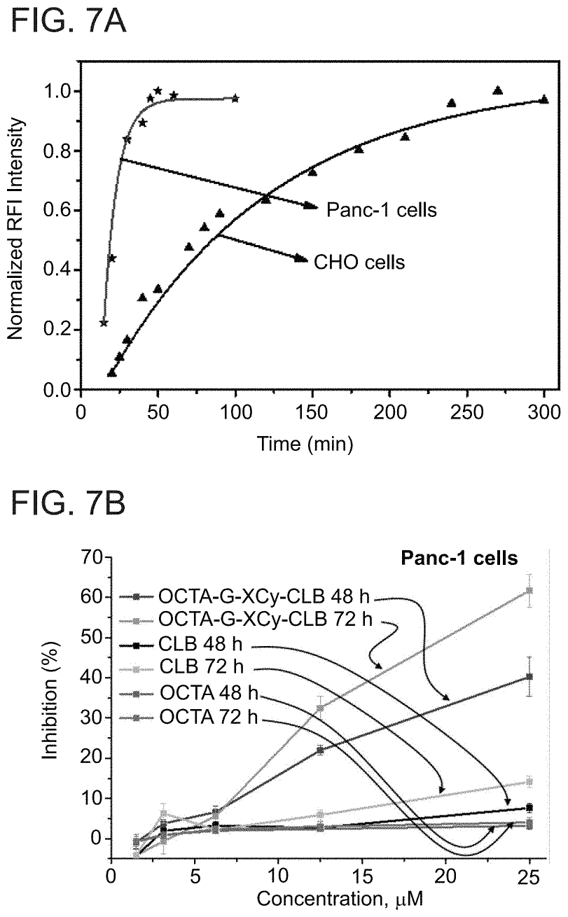

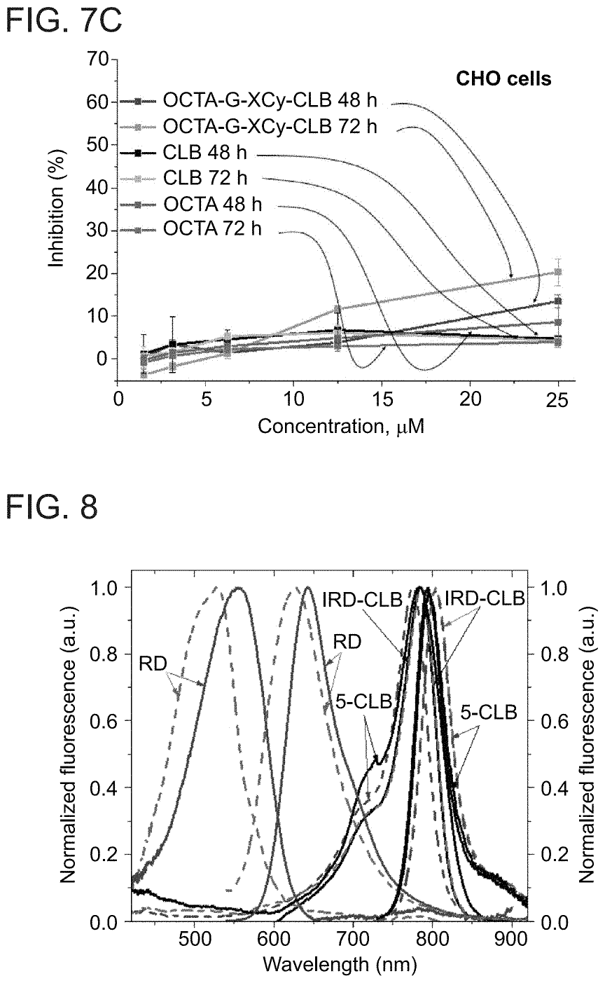

In the next step, the inventors investigated the performance of OCTA-G-XCy-CLB in real time monitoring of drug delivery and drug release into human pancreatic cancer cell line (Panc-1). As a negative control, Chinese hamster ovary cell line (CHO) was employed. Panc-1 contains overexpressed somatostatin receptors SSTR-2 and SSTR-5 while the CHO has the decreased number of these receptors. Panc-1 and CHO were incubated at 37° C. for 10 min with 10 μM OCTA-G-XCy-CLB in PBS pH 7.4 containing 3% DMSO and washed thoroughly with PBS pH 7.4 to remove excess conjugate. Then the fluorescence microscopy images were taken over time (FIGS. 7A, B). The fluorescence intensities for both cell lines gradually increase over time signaling the CLB release. The drug cleavage profiles for OCTA-G-XCy-CLB (FIG. 7C) indicate that the CLB release in Panc-1 is much faster (by about 6-fold) compared to CHO. The cleavage half-life (τ_{1/2}) in Panc-1 is about 25 min while for CHO τ_{1/2} about 2.5 h.

FIGS. 7A-C present drug (CLB) release profiles measured by relative fluorescence intensities (RFI) of selected Panc-1 and CHO cells (FIG. 7A), showing that the CLB cleavage half-life is τ_{1/2}˜25 min for Panc-1 and τ_{1/2} ˜2.5 h for CHO, and the cell inhibition of Panc-1 (FIG. 7B) and CHO (FIG. 7C) pre-treated with various concentrations of OCTA-G-XCy-CLB, free CLB and Free OCTA, whereas after the treatment, the cells were incubated for 24 h and 48 h at 37° C., cell inhibition was accessed using standard XTT assay, and the inhibition for each concentration point is represented by the mean±standard error for each independent experiment conducted in triplicate.

As can be seen in FIGS. 7A-C, cytotoxicity and specificity of OCTA-G-XCy-CLB was compared to that of CLB and OCTA on Panc-1 and CHO cell lines using a standard XTT cell survival assay. The obtained percentage Growth Inhibition (GI) indicates that OCTA-G-XCy-CLB preferably targets specific SSTR overexpressed on Panc-1 with significantly elevated growth inhibition compared to CHO (negative control) (FIGS. 7A-C). Notably, neither free OCT, nor free CLB exhibited detectible toxicity in both cell lines.

Thus, the inventors have demonstrated an anti-cancer targeted drug delivery system OCTA-G-XCy-CLB comprising NIR fluorescent switchable reporter XCy based on xanthene-cyanine dye attached to anticancer drug chlorambucil (CLB) and SSTR targeting peptide octreotide amide (OCTA). The imaging and cytotoxicity assays performed in Panc-1 cell line overexpressing somatostatin receptors SSTR-2 and SSTR-5 and CHO cell line containing a reduced number of these receptors demonstrate that OCTA-G-XCy-CLB can be employed as a potent and selective TDD system enabling real time monitoring of drug release in target tissues.

Materials and Methods:

All protected amino acids, resin and coupling reagents were purchased from Tzamal d-Chem Laboratories Ltd. All other chemicals were supplied by Alfa Aesar Israel or Sigma-Aldrich. Solvents were purchased from Bio-Lab Israel and used as is. Chemical reactions were monitored by TLC (Silica gel 60 F-254, Merck).

¹H NMR and ¹³C NMR spectra were measured at 300 K on a Bruker AvanceIII HD (¹H 400 MHz and ¹³C 100 MHz) spectrometer and a BBO probe equipped with a Z gradient coil. The samples were dissolved in various deuterated solvents according to their solubility.

LC/MS analyses were performed using an Agilent Technologies 1260 Infinity (LC) 6120 quadruple (MS), column Agilent SB-C18, 1.8 mm, 2.1×50 mm, column temperature 50° C., eluent water—acetonitrile (ACN)+0.1% formic acid.

HPLC purifications were carried out on an ECOM preparative system, with dual UV detection at 230 nm and 254 nm. A Phenomenex Gemini® 10 μm RP18 110 Å, LC 250×21.2 mm column was used. The column was kept at ambient temperature. Eluent A (0.1% TFA in water) and B (0.1% TFA in ACN) were used. A typical elution was a gradient from 100% A to 100% B over 35 min at a flow rate of 25 mL/min.

HRMS was measured in the ESI positive mode using an Agilent 6550 iFunel Q-TOF LC/MS.

Absorption spectra were recorded on a Jasco V-730 UV-Vis spectrophotometer and the fluorescence spectra were measured on Edinburgh FS5 spectrofluorometer. The absorption and fluorescence spectra were measured in 1-cm quartz cells at about 0.5 μM dye concentrations in PB 10 mmol buffer pH 7.4 at 25° C. and cell medium pH 7.4. Excitation wavelength was 650 nm. All the solutions were filtered in PVDF 0.45 μm filter before taking spectrum.

All the cell lines were cultured in an RPMI medium supplemented with 2 mM glutamine, 10% fetal bovine serum and with penicillin streptomycin (100 IU/ml of each). The cell culture growth medium and all its additives were purchased from Biological Industries, Bet-Ha'emek, Israel. All cell cultures were grown at a 37° C. incubator in an environment containing 6% CO2.

The Fluorescent images were acquired by Photometrics CoolSNAP HQ2 camera mounted on an Olympus iX81 fluorescent microscope. For the imaging, a cube comprising an ET620/60x bandpass excitation filter, ET700/75m bandpass emission filter and T661pxr dichroic filter were used.

To monitor drug release, Panc-1 and CHO cell lines were grown in six-well culture plates and then washed with PBS (pH 7.4) two times after that incubated for 10 min with 10 μM OCT(N)-G-XCy-CLB in PBS pH 7.4 containing 3% DMSO. After incubation, the samples washed thoroughly with PBS (pH 7.4) two times to remove excess conjugate. The washed cell samples were resuspended in PBS and immediately analyzed the fluorescence changes by using fluorescent microscope. All images were taken at 37° C. incubator in an environment containing 6% CO2.

The band intensities in a representative experiment were quantified by creating region of interest (ROI) around each image and the relative fluorescence intensity of each sample was measured (via the “Measure” function) with Image J software.

The cytotoxicity of the peptide-drug conjugates was determined by measuring the mitochondrial enzyme activity, using a commercial XTT assay kit. All samples were prepared in PBS pH 7.4 contained 3% DMSO. The Cells were cultured in micro wells at a concentration 5-10×104 cells/well. The cells were washed and fresh cell medium containing different concentrations (up to 25 μM) of the conjugates were added and the cells were incubated for 10 minutes. The cultures were washed and then given a fresh medium and cultured for 48 hours and 72 hours. At the end of the second incubation, XTT reagent was added and the cells were re-incubated for additional 2-4 hours. During that time the absorbencies in the wells were measured with a TECAN Infinite M200 ELISA reader at 480 nm and 680 nm. The difference in the absorbencies measurements at these two wavelengths was used for calculating the percentage Growth Inhibition (GI) in test wells compared to two controls: cells that were exposed to the medium and solvent, and those which were exposed to a solvent-free medium. All the tests were done in triplicate.

### Chemical Synthesis and Characterization:

Synthesis of Cyanine (Cy): Cyanine was synthesized according to the procedure described by Luo, S. et al. [Adv. Funct. Mater., 2016, 26, pp. 2826-2835] with slight modification; at a yield of 66%.

### Synthesis of (E)-1-(5-carboxypentyl)-2-(2-(6-hydroxy-2,3-dihydro-1H-xanthen-4-yl)vinyl)-3,3-dimethyl-3H-indol-1-ium (XCy)

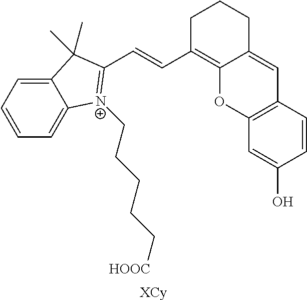

K₂CO₃ (276 mg, 2 mmol) and resorcinol (220 mg, 2 mmol) were dissolved in acetonitrile (20 mL) and stirred for 15 min under N₂ atmosphere. The above mixture was added to a solution of Cy (683 mg, 1 mmol) in acetonitrile (15 mL) and stirred for 8 hours at 50° C. The reaction was monitored by TLC. After reaction, the solvent was evaporated under reduced pressure and the crude product was purified by using silica gel column chromatography (DCM/Methanol=90:10). The product XCy (295 mg, 61% yield) was obtained as a blue solid. ¹H NMR of compound XCy (400 MHz, CD₃OD) δ (ppm): 8.56 (d, J=14.8 Hz, 1H), 7.53 (d, J=7.5 Hz, 1H), 7.4 (d, J=3.8 Hz, 2H), 7.32-7.27 (m, 3H), 6.74 (d, J=2.3 Hz, 1H), 6.71 (s, 1H), 6.31 (d, J=14.8 Hz, 1H), 4.22 (t, j=7.4 Hz, 2H), 2.64 (t, J=5.8 Hz, 2H), 2.58 (t, J=6.0 Hz, 2H), 2.19 (t, J=7.2 Hz, 2H), 1.83-1.78 (m, 4H), 1.69 (s, 6H), 1.63-1.61 (m, 2H), 1.45-1.41 (m, 2H). ¹³C NMR (100 MHz, CD₃OD), 178.2, 163.96, 163.58, 156.17, 146.39, 143.11, 142.98, 136.36, 130.4, 130.11, 127.93, 127.36, 123.68, 116.27, 116.03, 115.61, 113.48, 107.64, 103.73, 102.9, 51.65, 45.79, 35.24, 29.87, 28.37 (2C), 28.3, 27.31, 25.82, 25.02, 21.63 MS of compound XCy: calculated 484.2, C₃₁H₃₄NO₄⁺ and found LC-MS: m/z 484.0.

### Synthesis of (E)-2-(2-(6-((4-(4-(bis(2-chloroethyl)amino)phenyl)butanoyl)oxy)-2,3-dihydro-1H-xanthen-4-yl)vinyl)-1-(5-carboxypentyl)-3,3-dimethyl-3H-indol-1-ium (XCy-CLB)

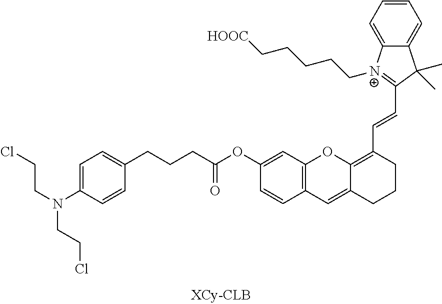 [SMILES: CC1(C)C(/C=C/C2=C3Oc4cc(OC(=O)CCCc5ccc(N(CCCl)CCCl)cc5)ccc4C=C3CCC2)=[N+](CCCCCC(=O)O)c2ccccc21]

Chlorambucil (121 mg, 0.4 mmol) and SOCl₂ (58 μL, 0.8 mmol) were stirred in DCM (15 mL) under N₂ atmosphere in 0° C. for 3 hours. After 3 hours Et₃N (167 μL, 1.2 mmol) was added to the mixture via syringe. A mixture of XCy (100 mg, 0.20 mmol) and DMAP (24 mg, 0.20 mmol) in DCM (20 mL) added to the above mixture via syringe and stirred for 6 hours in 50° C. The reaction was monitored by TLC. After reaction, the solvent was evaporated under reduced pressure and the crude product was purified by using silica gel column chromatography (DCM/Methanol=90:10). The product XCy-CLB (93 mg, 60% yield) was obtained as a blue solid. ¹H NMR of compound XCy-CLB (400 MHz, CD₃OD) δ (ppm): 8.68 (d, J=15 Hz, 1H), 7.57 (m, 1H), 7.52 (m, 1H), 7.46 (m, 1H), 7.4 (m, 2H), 7.22 (s, 1H), 7.18 (d, J=2.1 Hz, 1H), 7.02 (d, J=8.7 Hz, 2H), 6.94 (dd, J=8.4 Hz, 1H), 6.61 (d, J=8.8 Hz, 2H), 6.52 (d, J=15 Hz, 1H), 4.32 (t, J=7.5, 2H), 3.65-3.61 (m, 4H), 3.58-3.55 (m, 4H), 2.69 (t, J=6.7 Hz, 2H), 2.63 (t, J=6 Hz, 2H), 2.58-2.52 (m, 4H), 2.21 (t, J=7.2 Hz, 2H), 1.93 (t, J=7.3 Hz, 2H), 1.87-1.82 (m, 4H), 1.72 (s, 6H), 1.62-1.59 (m, 2H), 1.44-1.41 (m, 2H). ¹³C NMR (100 MHz, CD₃OD), 180.48, 173.31, 161.78, 154.65, 154.6, 147.92, 146.34, 144.01, 142.88, 132.91, 131.51, 131.41, 131, 130.88 (2C), 130.5, 129.58, 129.19, 124.03, 121.19, 120.52, 116.19, 114.6, 113.77 (2C), 110.76, 106.64, 54.7 (2C), 52.59, 46.56, 41.88 (2C), 35.13, 34.51, 30.88, 30.43, 28.8, 28.28 (2C), 27.91, 27.46, 25.89, 25.16, 21.66 MS of compound XCy-CLB: calculated 769.3170, C₄₅H₅₁C₁₂N₂O₅⁺ and found HRMS: m/z 749.3174.

Solid phase synthesis of Octreotide amide-GABA (OCTA-G): The synthesis of the cyclic peptide OCTA-G was done according to the previously described [Redko, B. et al., Biopolymers, 2015, 104(6), pp. 743-52; Gilad, Y. et al., Bioorg Med Chem. 2016, 24(2), pp. 294-303; Redko, B. et al., Oncotarget. 2017, 8(1), pp. 757-′768; Gilad, Y. et al., Eur J Med Chem., 2014, 85, pp. 139-146; and Gellerman, G. et al., J Pept Res., 2001, 57(4), pp. 277-291]. Rink Amide resin (0.65 mmol/g) was placed in a sintered glass bottom and swelled in NMP by agitation overnight. The Fmoc group was removed from the resin by treatment with 20% piperidine/NMP (2×15 minutes). The completion of the removing of Fmoc was monitored by ninhydrin test (blue). After washing the resin with NMP (7×2 min), a mixture of Fmoc-Thr(Trt)-OH (2 eq.), DIPEA (8 eq.) and PyBOP (2 eq) in NMP was added. The reaction was carried out for 1.5 h at room temperature. After coupling, the peptidyl resin was washed with NMP (5×2 min). The completion of the reaction was monitored by ninhydrin test (yellow). Then a linear SPPS was applied using standard Fmoc procedures introducing the amino acids in the following order: Fmoc-Cys(Acm)-OH, Fmoc-Thr(Trt)-OH, Fmoc-Lys(Boc)-OH, Fmoc-DTrp(Boc)-OH, Fmoc-Phe-OH, Fmoc-Cys(Acm)-OH, Fmoc-DPhe-OH, Fmoc-GABA-OH. All the couplings were performed in NMP with 2-fold excess of amino acid, DIPEA (8 eq) and PyBOP (2 eq.) for activation. Each coupling cycle was conducted for 1.5 h. The completion of each coupling reaction and Fmoc removal were monitored by the ninhydrin test. After the coupling of the last amino acid, the sequence was cyclized by 12 (10 eq.) in DMF:H₂O (4:1) for 2 hours, washed (DMF:H₂O (4:1) 5×2 min, DCM 5×2 min, chloroform 5×2 minutes, 2% ascorbic acid in DMF, DMF 5×2 min) and N-terminus Fmoc was deprotected using regular procedure yielding the cyclic peptidyl residue (OCTA-G) on Rink Amide resin ready for next conjugation.

Synthesis of OCTA-G-XCy-CLB:

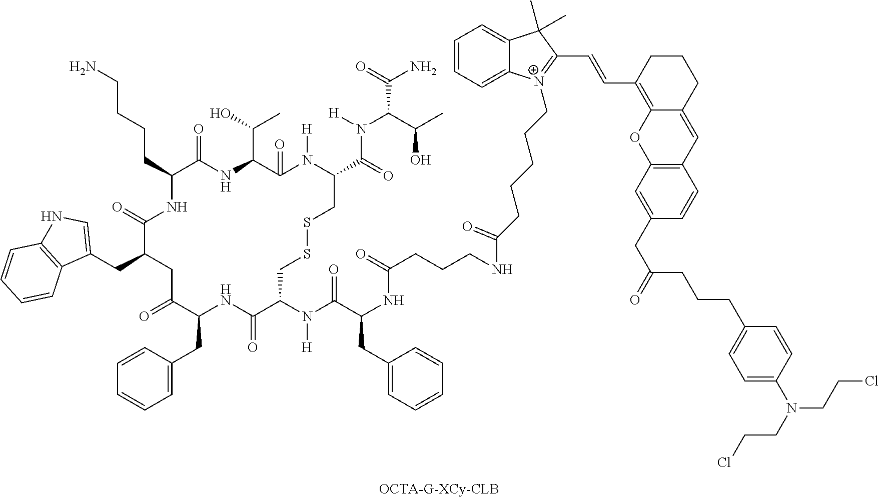 [SMILES: C[C@@H](O)[C@H](NC(=O)[C@@H]1CSSC[C@H](NC(=O)[C@H](Cc2ccccc2)NC(=O)CCCNC(=O)CCCCC[N+]2=C(/C=C/C3=C4Oc5cc(CC(=O)CCCc6ccc(N(CCCl)CCCl)cc6)ccc5C=C4CCC3)C(C)(C)c3ccccc32)C(=O)N[C@@H](Cc2ccccc2)C(=O)C[C@@H](Cc2c[nH]c3ccccc23)C(=O)N[C@@H](CCCCN)C(=O)N[C@@H]([C@@H](C)O)C(=O)N1)C(N)=O]

A mixture of XCy (35 mg, 1.2 eq.), HATU (35 mg, 1.5 eq.) and DIPEA (50 μL, 5 eq) in NMP was stirred for preactivation for 2 min and consequently added to the OCTA-G (immobilized on solid phase) (200 mg, 1 eq). The reaction was carried out for 1 h at room temperature. After coupling (ninhydrin test-yellow), the peptidyl resin was washed with NMP (5×2 min) and DCE (5×2 min). Thereafter, carboxyl function of the drug CLB (37 mg, 2 eq.) was activated by treatment with BTC (26 mg, 1.5 eq.) and collidine (45 μL, 6 eq.) in 4 ml DCE for 1 min and added to the peptidyl resin. The reaction was carried out for 1 hour. After coupling, the peptidyl resin was washed with DCE (5×2 min).

Cleavage procedure for Rink Amide resin: The OCTA-G-XCy-CLB on the peptidyl-resin was treated with the cold TFA cocktail (95% TFA, 2.5% TIS, 2.5% H₂O) for 1.5 hours. The solvent was removed under a gentle flow of N₂ and then the crude was precipitated from Et₂O. The obtained crude OCTA-G-XCy-CLB were purified by preparative HPLC, pure fractions were lyophilized yielding (3.7 mg, 1.7, 2.75% yield). MS of compound OCTA-G-XCy-CLB: calculated 1867.7949, C₉₈H₁₂₁Cl₂N₁₄O₁₅S₂⁺ and found HRMS: m/z 1868.79 [M+H⁺]. LCMS purity chromatogram confirmed yield, LCMS mass spectrum 934.7 (M/2+2H+).

### Example 2

### Theranostic System for Monitoring of Targeted Drug Delivery

Below is an exemplary embodiment of a conjugate of anticancer drug with cancer-specific carrier and fluorescent dye to form a theranostic system, which enables real time monitoring of targeted drug delivery (TDD). Since the fluorescence signal from the dye is affected by the light absorption and scattering in the body, the quantitative determination of the drug release degree in target tissues is a challenging task. In the example below, ratiometric measurements utilizing two fluorescence signals of different wavelengths are utilized to improve quantitation in biological matter; thus, a switchable, long-wavelength heptamethine cyanine dye IRD is demonstrated as a ratiometric fluorescent TDD monitoring. This dye has been coupled to targeting peptide octreotide amide (OctA) and, via a triggering biodegradable ester bond, has been bound to the anticancer drug chlorambucil (CLB) to form a theranostic conjugate, according to some embodiments of the present invention. The drug-bound dye has been shown to absorb and emits light in the near-infrared (NIR) region but upon the environment-mediated drug release its fluorescence turns red. Comparison of these two signals enables ratiometric measurements of drug release. Advantage of the developed theranostic system for the ratiometric fluorescence TDD monitoring has been demonstrated in the example of a human pancreatic cancer cell line PANC-1.

### Materials and Methods:

All protected amino acids, resin and coupling reagents were purchased from Tzamal d-Chem Laboratories Ltd. All other chemicals were supplied by Alfa Aesar Israel or Sigma-Aldrich. Octreotide was purchased from Glentham life sciences Solvents were purchased from Bio-Lab Israel and used as is. Chemical reactions were monitored by TLC (Silica gel 60 F-254, Merck).

¹H NMR and ¹³C NMR spectra were measured at 300 K on a Bruker AvanceIII HD (¹H 400 MHz and ¹³C 100 MHz) spectrometer and a BBO probe equipped with a Z gradient coil. The samples were dissolved in various deuterated solvents according to their solubility.

LC/MS analyses were performed using an Agilent Technologies 1260 Infinity (LC) 6120 quadruple (MS), column Agilent SB-C18, 1.8 mm, 2.1×50 mm, column temperature 50° C., eluent water—acetonitrile (ACN)+0.1% formic acid.

HPLC purifications were carried out on an ECOM preparative system, with dual UV detection at 230 nm and 254 nm. A Phenomenex Gemini® 10 μm RP18 110 Å, LC 250×21.2 mm column was used. The column was kept at ambient temperature. Eluent A (0.1% TFA in water) and B (0.1% TFA in ACN) were used. A typical elution was a gradient from 100% A to 100% B over 35 minutes at a flow rate of 25 mL/min.

HRMS was measured in the ESI positive mode using an Agilent 6550 iFunel Q-TOF LC/MS.

PANC-1 cell line was cultured in an RPMI medium supplemented with 2 mM glutamine, 10% fetal bovine serum and with penicillin streptomycin (100 IU/ml of each). (The cell culture growth medium and all its additives were purchased from Biological Industries, Bet-Ha'emek, Israel). The cell culture was grown at a 37° C. incubator in an environment containing 6% CO2.

The Fluorescent images were acquired by Photometrics CoolSNAP HQ2 camera mounted on an Olympus iX81 fluorescent microscope. For the Red channel a cube comprising a ET560/40x bandpass excitation filter, ET630/75m bandpass emission filter and T5851pxr dichroic filter was used. For the NIR channel a cube comprising a ET740/40x bandpass excitation filter, ET7801p long pass emission filter and T7701pxr dichroic filter was used. To monitor drug release, Panc-1 cell line was grown in six-well culture plates and then washed with PBS (pH 7.4) two times and then incubated 10 minutes with 5-CLB 10 μM solution in PBS (for better solubility PBS with 3% DMSO was used). After incubation, the samples washed thoroughly with PBS (pH 7.4) two times to remove excess conjugate and the fluorescence changes immediately measured by using fluorescent microscope. All images were taken at 37° C. incubator in an environment containing 6% CO₂.

The band intensities in a representative experiment were quantified by measurement of related fluorescence intensity for each image (via the “Measure” function) with Image J software.

### Chemical Synthesis and Characterization

In the example below, the TDD system comprises an anti-cancer drug chlorambucil (CLB) performing as DNA alkylator, a peptide carrier octreotide amide (OctA), which specifically binds to somatostatin receptors overexpressed in various human tumor cells, and a switchable cyanine dye (IRD) that enables the ratiometric fluorescence monitoring of targeted drug delivery (see, FIG. 2B).

The IRD dye (Scheme 4 and Scheme 5 below) belongs to heptamethine cyanines containing a triggering hydroxyl group in the central cyclohexene moiety. Several dyes of this series have recently been introduced and spectral properties thoroughly investigated [Pascal, S. et al., J. Phys. Chem. A, 2014, 118, 4038-4047].

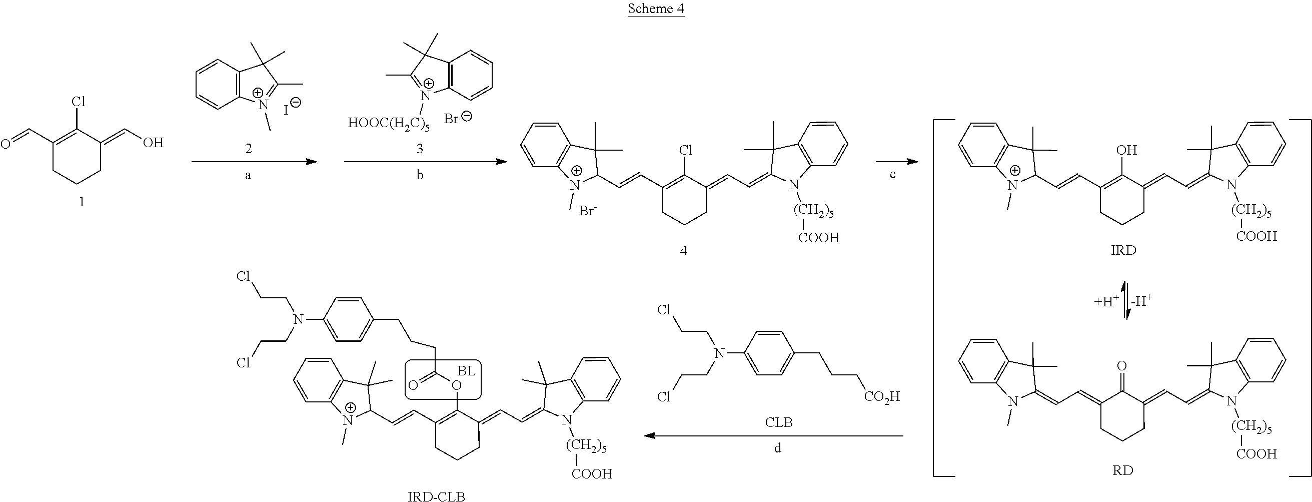

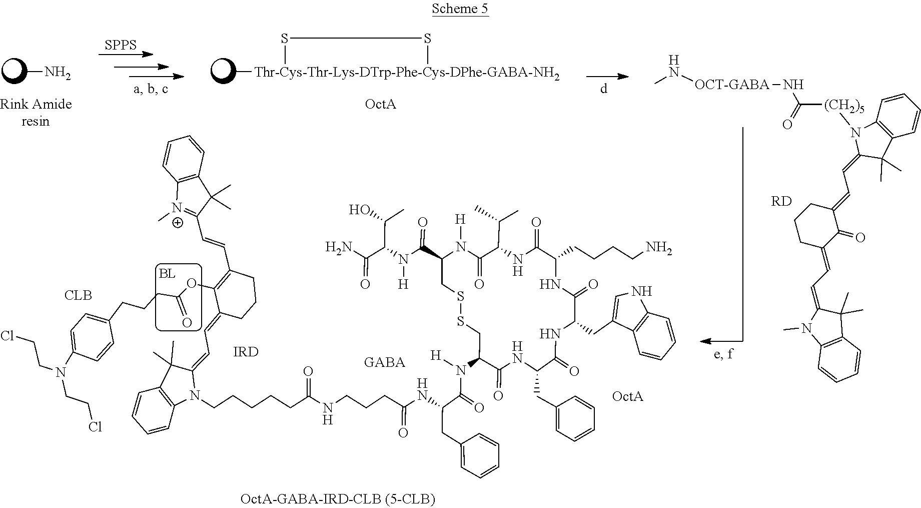

Scheme 4 presents the synthesis of RD and IRD-CLB, wherein BL is a biodegradable ester linker, and the reaction conditions are: a—toluene/dioxane (1:1, v/v), reflux 4 h; b toluene/dioxane/NMP (5:5:2, v/v/v), reflux 4 hours; c—NaOAc in DMF, 90° C.; 2 hours; d BTC, collidine, DCE, 2 hours, RT. Scheme 5 presents the synthesis of 5-CLB, wherein BL is a biodegradable ester linker, and the reaction conditions are: a—Fmoc removal: 20% piperidine in NMP; b—Coupling: PyB OP (2 eq), AA (2 eq), DIPEA (8 eq), 1.5 hours; c—Cyclization step: 12 (10 eq.) in DMF—H₂O (4:1, v/v), 2 hours; d—HATU, DIPEA, RD, NMP; e—BTC, collidine, DCE; CLB; f—TFA.

In the proof of concept provided herein, it has been demonstrated that these dyes can be utilized for certain ratiometric sensing applications such as the determination of cysteine, hydrogen sulfide, hydrogen polysulfides and superoxide anion in living cells. When a sensing group is attached to the cyclohexenol oxygen through a cleavable ester linker, these dyes absorb and emit within the NIR region at about 770-810 nm (“hydroxy” form). Upon the triggering group release, the dyes transform into the “keto” form exhibiting absorption in the green, around 515-530 nm, and a red emission at about 605-625 nm. It has been hypothesized by the present inventors, that these fluorophores can also be used for the ratiometric fluorescence measurements in TDD monitoring.

As compared to many other sensing applications, the dye used in a TDD conjugate must have a single reactive functionality for binding to targeting carrier. In the example below, a new asymmetric, switchable NIR dye IRD has been synthesized and isolated in its deprotonated, red emitting “keto” form (“RD”, see, Scheme 4). This dye contains a carboxyl function facilitating its coupling to a targeting carrier. The synthesis was carried out starting from di-aldehyde (Compound 1) that was subsequently reacted with quaternized indolenines Compound 2 and Compound 3 to chlorinated cyanine dye Compound 4 (one-pot reaction), which was then converted to the desired RD dye.

Starting from RD, the TDD conjugate OctA-GABA-IRD-CLB (5-CLB) was synthesized, comprising the reporter dye IRD, bound to the anticancer drug CLB by means of a biodegradable ester linker (BL), and to the targeting peptide OctA via a non-cleavable GABA linker (See, Scheme 5). For comparison, to investigate impact of the peptide on the spectral properties and drug release profiles, IRD-CLB conjugate (Scheme 4) was also obtained. The last one was synthesized by the condensation of RD with CLB pre-activated by treatment with triphosgene and collidine. The 5-CLB conjugate was obtained in two steps from Rink amide resin (RAM) loaded with OctA-GABA peptidyl tether using a solid state peptide synthesis (SSPS) (Scheme 5). A GABA linker increasing the peptide-dye distance was used to facilitate their conjugation. In the next step, RD was coupled to RAM immobilized with OctA-GABA in presence of HATU and DIEA and then pre-activated CLB was bound to OctA-GABA-IRD followed by the TFA activated cleavage of the aimed TDD conjugate from the solid phase.

The spectral properties of the dye RD and its conjugates IRD-CLB and 5-CLB were measured at the concentrations (c) of 0.6 μM in 10 mM phosphate buffer pH 7.5 (PB) and cell medium pH 7.5 (CM), both containing 20% of acetonitrile (ACN) to improve solubility of the compounds. The absorption and fluorescence spectra are shown in FIG. 8, while the spectral characteristics are presented in Table 2.

FIG. 8 presents a Normalized absorption and emission spectra of RD, IRD-CLB, and 5-CLB measured at c=0.6 μM in PB (solid line) and CM (dashed line), whereas the excitation wavelength was 532 nm for RD and 720 nm for IRD-CLB and 5.

Table 2 presents the absorption (λₘₐₓAb) and emission (λₘₐₓFl) maxima, extinction coefficients (ε) and fluorescence quantum yields (Φ_{F}) of the RD dye and IRD-CLB and 5-CLB conjugates measured at 25° C. in 10 mM phosphate buffer pH 7.5 (PB) and cell medium pH 7.5 (CM).

TABLE 2

| | PB:ACN (4:1, v/v) | | | | CM:ACN (4:1, v/v) | | |
|---|---|---|---|---|---|---|---|
| Dye/ | λₘₐₓAb | ε | λₘₐₓFl | Φ_{F} | λₘₐₓAb | λₘₐₓFl | Φ_{F} |
| Conjugate | (nm) | (M⁻¹cm⁻¹) | (nm) | (%) | (nm) | (nm) | (%) |
| RD | 555 | 57,000 | 643 | 9.9 ± | 526 | 626 | 2.6 ± |
| | | | | 0.8 | | | 0.1 |
| IRD-CLB | 775 | 160,000 | 795 | 2.9 ± | 785 | 805 | 6.3 ± |
| | | | | 0.3 | | | 0.5 |
| 5-CLB | 783 | n.d. | 796 | 1.5 ± | 787 | 805 | 5.9 ± |
| | | | | 0.2 | | | 0.4 |

Importantly, both forms (RD and IRD) of the dye are fluorescent but have substantially different absorption and emission maxima. RD measured in PB has the absorption band at 555 nm with the extinction coefficient of about 57,000 M⁻¹ cm⁻¹; the emission maximum is 643 nm and the fluorescence quantum yield 9.9% (see, Table 2). In cell medium (CM), which is a more hydrophobic and less polar media due to the presence of proteins, the spectral bands are blue-shifted by 29 nm and 17 nm, respectively, and the quantum yield is about 4 fold decreased (O_{F}=2.6%). Conjugation of RD with CLB to form IRD-CLB causes a pronounced red-shift of the absorption and emission bands to the NIR region (by 220 nm and 152 nm, respectively, in PB and even more, 259 nm and 178 nm in CM) and a 2.8 fold increase in the extinction coefficient (ε=160,000 M⁻¹ cm⁻¹). The quantum yield after conjugation decreases by factor of 3.4 (IRD-CLB) and 6.6 (5-CLB) in PB but increases by factor of 2.4 in CM. Binding of IRD-CLB to OctA-GABA to form conjugate 5-CLB causes a minor effect on the spectral bands.

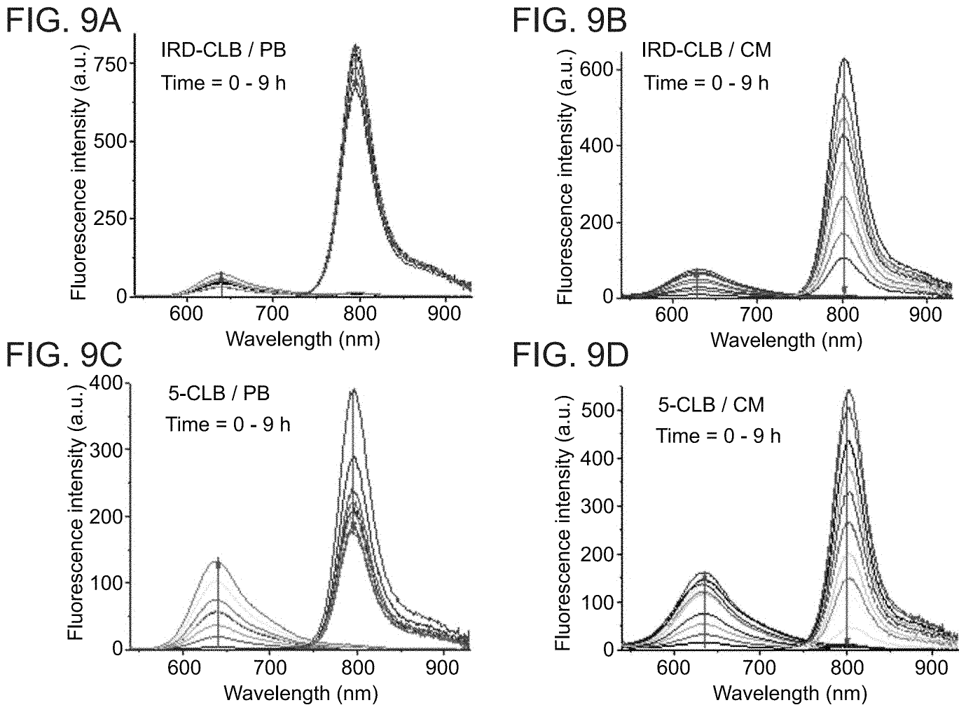

The biodegradable ester linker (BL) in IRD-CLB and 5-CLB can be hydrolyzed to release free CLB and, accordingly, IRD turns into RD, which is accompanied by a noticeable change in the spectral properties. The time-dependent emission spectra were recorded in PB and CM (see, FIGS. 9A-D) using the excitation wavelengths 720 nm and 532 nm. The first one enabled monitoring of the decrease in the concentration of IRD-CLB and 5-CLB, while the second one—the increase in the concentration of RD and OctA-RD. Only the NIR fluorescence of IRD-CLB and 5-CLB was detected, when excited at 720 nm; and only the red fluorescence of RD and OctA-RD was detected, when excited at 532 nm. No spill over between these two emission spectra was observed: IRD-CLB and 5-CLB exhibited no detectable emission, when excited at 532 nm, and therefore no compensation was required to quantify concentration of the conjugated and released drug molecules.

FIGS. 9A-D present a plot showing the time-dependent fluorescence spectra at T=25° C. of IRD-CLB (FIGS. 9A, B) and 5-CLB (FIGS. 9C, D) in PB (FIGS. 9A, C) and CM (FIGS. 9B, D).

Upon the hydrolytic cleavage, the NIR emission band (F_{NIR}) related to IRD decreases and the red emission band (F_{Re}d) of RD increases (FIGS. 9A-D). Based on the time-dependent emission spectra, the drug cleavage profiles for IRD-CLB and 5-CLB were obtained (FIG. 9A) and the drug release rates were quantified by the half-lives (τ_{1/2}). For IRD-CLB τ_{1/2} was found to be about 25 hours in PB and 1.3 hours in CM, and for 5-CLB τ_{1/2} was ˜2.4 hours and ˜2.0 hours, respectively. This indicates that the conjugation of OctA with IRD-CLB to form 5-CLB noticeably accelerates the CLB release in PB but slows down it in CM.

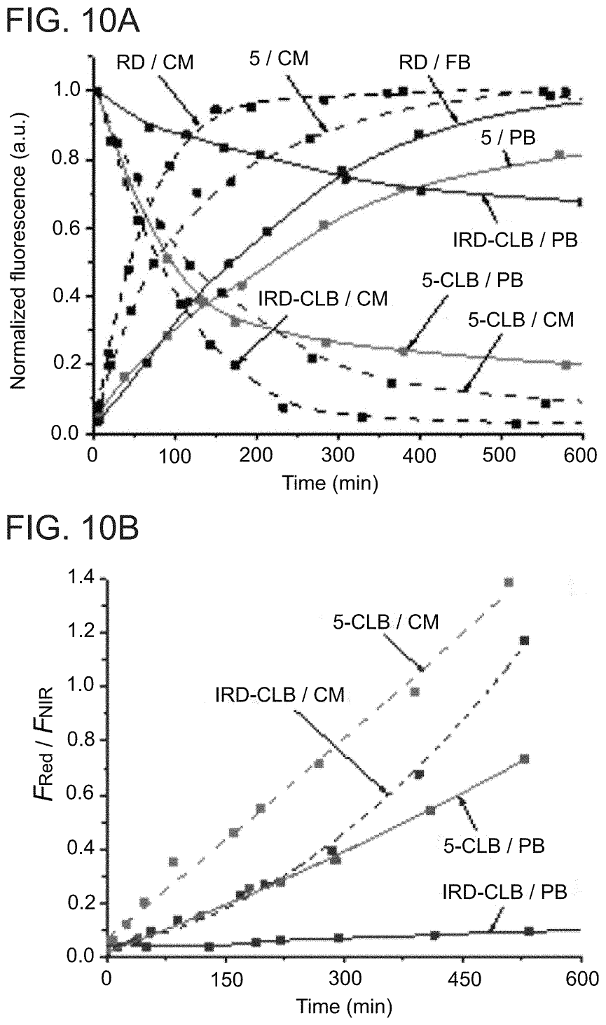

The intensity of each fluorescence signal (F_{Re}d and F_{NIR}) is dependent not only on the degree of hydrolysis but also on the initial concentration of the IRD-CLB or 5-CLB conjugate. In addition, when imaging through body, the fluorescence signal is affected by the light path depth. Because both signals, F_{Re}d and F_{NIR}, originate from the same dye molecule (existed in IRD or RD form), the ratiometric measurements based on the comparison of these two signals (F_{Red}/F_{NIR}) enable exceptional determination of the cleavage degree independently of the initial concentration of the dye-drug conjugate and compensation of the light path effect. The changes in the F_{Red}/F_{NIR} ratios measured in PB and CM are shown in FIG. 10B. The presented ratiometric curves, as compared to the intensity based profiles in FIG. 10B, are unaffected by concentration and the light path depth and therefore can be utilized to estimate the percentage of the drug molecules released from the conjugates. By taking the ratio between the two intensities over time, the concentration of the released CLB drug molecules can be directly correlated to these kinetics curves.

FIGS. 10A-B present comparative plots showing the CLB cleavage profiles (FIG. 9A) and ratiometric curves (F_{Red}/F_{NIR}) (FIG. 9B) for IRD-CLB and 5-CLB (c=0.6 μM) measured in PB (solid line) and CM (dashed line) at 25° C. after incubation at 37° C.

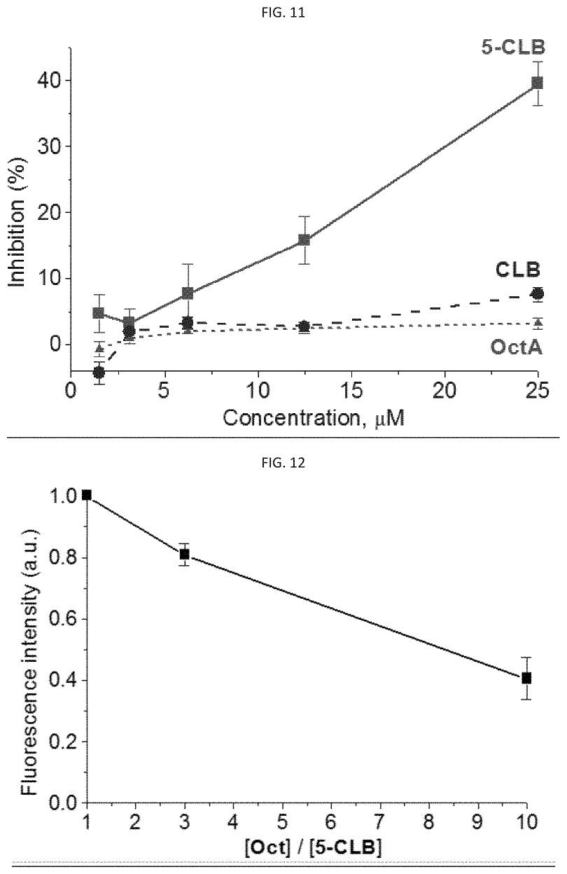

Application of the 5-CLB conjugate equipped with targeting peptide OctA was studied in the TDD monitoring using PANC-1 cancer cell line that overexpress SSTR-2 and SSTR-5 receptors. Efficient delivery of 5-CLB by the conjugated OctA to PANC-1 was verified by the XTT assay. Cytotoxicity of 5-CLB was compared to that of CLB or OctA added separately. The compounds were incubated with cells for 10 min at 37° C. at concentrations up to 25 μM, and subsequently were washed out, and growth inhibition compared to naïve/native cells was assessed in 24 hours. The 5-CLB caused dramatically higher growth inhibition compared to CLB or OctA, indicating that the OctA peptide provides potent targeting of 5-CLB conjugate to PANC-1 (FIG. 11).

Synthesis of 2-chloro-3-(hydroxymethylene)cyclohex-1-ene-1-carbaldehyde using nuaternized indolenines, was reported elsewhere [Prasad, P. R. et al., J. Org. Chem. 2016, 81, 3214-3226; Ong, M. J. H. et al., Org. Lett. 2016, 18, 5122-5125; and Tan, X. et al., Biomaterials 2012, 33, 2230-2239].

2-(−2-(−3-(2-(−1-(5-carboxypentyl)-3,3-dimethylindolin-2-ylidene)ethylidene)-2-chlorocyclohex-1-en-1-yl)vinyl)-1,3,3-trimethyl-3H-indol-1-ium bromide: Quaternized indolenine (0.9 g, 3.2 mmol, 1.0 eq.) was dissolved at 80° C. in 50 mL of a mixture of toluene and dioxane (1:1, v/v). Compound 1 (0.55 g, 3.2 mmol, 1 eq.) was added to the obtained solution and stirred at 80° C. for 4 h. The solution was cooled to RT and filtered out, the product was precipitated from solution with 100 mL of hexane. Precipitate was dissolved in 50 mL of toluene and dioxane (1:1, v/v). Next, the quaternized indolenine (1.1 g, 3.2 mmol, 1.0 eq.) was dissolved in 10 ml of NMP, added to the reaction mixture and heated at 80° C. for 4 hours. The resulted reaction mixture was cooled to RT and the product was precipitated with 100 mL of diethyl ether to give cyanine (1.2 g, 1.8 mmol, 56%) as a green solid. The product was used in the next step without any purification. For NMR and MS analysis 50 mg of cyanine was purified by using silica gel column flash chromatography (DCM/Methanol=90:10), yielding 17 mg of desired pure product.

¹H NMR (400 MHz, CDCl₃) δ=8.36 (d, J=13.7, 1H), 8.32 (d, J=14.1, 1H), 7.45-7.32 (m, 4H), 7.25-7.14 (m, 4H), 6.22 (d, J=14.2, 1H), 6.18 (d, J=14.0, 1H), 4.15 (t, J=7.6, 2H), 3.71 (s, 3H), 2.72 (t, J=6.2, 4H), 2.53 (t, J=7.2, 2H), 1.98 (p, J=6.4, 2H), 1.87 (p, J=7.5, 2H), 1.78 (p, J=7.5, 2H), 1.71 (s, 11H), 1.62-1.50 (m, 3H). ¹³C NMR (101 MHz, CDCl₃) δ=176.12, 172.75, 172.72, 150.89, 144.92, 144.31, 142.97, 142.19, 141.20, 140.98, 129.10, 128.94, 127.98, 127.96, 125.62, 125.32, 122.38, 122.24, 111.20, 110.79, 101.68, 101.42, 49.54, 49.26, 44.78, 34.57, 32.13, 28.27, 28.22, 27.09, 26.77, 26.72, 26.37, 24.64, 20.82. HRMS m/z (ESI+) C₃₇H₄₄ClN₂O₂⁺ calculated [M⁺] 583.3086, found 583.30995.

6-(−3,3-dimethyl-2-(−2-(−2-oxo-3-(2-(−1,3,3-trimethylindolin-2-ylidene) ethylidene) cyclohexylidene) ethylidene) indolin-1-yl)hexanoic acid (RD): 2-(−2-(−3-(2-(−1-(5-carboxypentyl)-3,3-dimethylindolin-2-ylidene)ethylidene)-2-chlorocyclohex-1-en-1-yl)vinyl)-1,3,3-trimethyl-3H-indol-1-ium bromide (1 g, 1.5 mmol, 1 eq) form the previous step was dissolved in 50 ml of dry DMF than dry sodium acetate (0.37 g, 4.5 mmol, 3 eq) was added, the mixture was heated to 90° C. for 2 h. After reaction completed (LC-MS monitoring), the solvent was evaporated under reduced pressure, dissolved in DCM and extracted with water, organic phase was dried with anhydrous sodium sulfate and the crude product was purified by using silica gel column flash chromatography (DCM/Dioxane=85:15). The product RD was obtained after evaporation as red-pink oil (0.27 g, 0.48 mmol) Yield 32%.

¹H NMR (400 MHz, MeOD) δ=8.19 (d, J=13.0 Hz, 2H), 7.26 (d, J=7.5 Hz, 2H), 7.21 (t, J=7.7 Hz, 2H), 6.94 (t, J=7.4 Hz, 2H), 6.87 (d, J=7.9 Hz, 2H), 5.59 (d, J=13.3 Hz, 1H), 5.53 (d, J=13.2 Hz, 1H), 3.85-3.75 (m, 2H), 3.28 (s, 3H), 2.61 (t, J=5.8 Hz, 4H), 2.31 (t, J=7.2 Hz, 2H), 1.91-1.81 (m, 2H), 1.80-1.67 (m, 4H), 1.65 (s, 12H), 1.47 (m, 2H). MS (ESI+) m/z of compound RD: calculated 564.3, C₃₇H₄₄N₂O₃ and found LC-MS: m/z 565.0 (M+1).

2-(−2-(−2-((4-(4-(bis(2-chloroethyl)amino)phenyl)butanoyl)oxy)-3-(2-(−1-(5-carboxypentyl)-3,3-dimethylindolin-2-ylidene)ethylidene)cyclohex-1-en-1-yl)vinyl)-1,3,3-trimethyl-3H-indol-1-ium (IRD-CLB): RD (40 mg, 0.07 mmol, 1 eq) was dissolved in 3 ml of DCE than solution of CLB (43 mg, 0.14 mmol, 2 eq), BTC (25 mg, 0.1 mmol, 0.7 eq) and collidine (55 μl, 0.42 mmol, 6 eq) in 3 ml DCE was added. Reaction mixture was stirred for 2 h, evaporated and purified by RP chromatography; pure fractions were lyophilized to obtain 23 mg of pure IRD-CLB, Yield 67%.

¹H NMR (400 MHz, Methanol-d₄) δ=7.68 (d, J=14.2, 1H), 7.64 (d, J=13.7, 1H), 7.44-7.32 (m, 4H), 7.30-7.19 (m, 4H), 7.15 (d, J=8.8, 2H), 6.76 (d, J=9.0, 2H), 6.14 (d, J=14.6, 1H), 6.11 (d, J=14.1, 1H), 4.08 (t, J=7.4, 2H), 3.74 (d, J=7.4, 4H), 3.63 (t, J=6.6, 4H), 3.59 (s, 3H), 2.82 (d, J=8.0, 2H), 2.71 (t, J=7.2, 2H), 2.63 (t, J=6.3, 4H), 2.26 (t, J=7.2, 1H), 2.09 (t, J=8.3, 2H), 1.91 (p, J=5.9, 2H), 1.78 (p, J=7.6, 2H), 1.64 (p, J=7.3, 2H), 1.50 (s, 6H), 1.49 (s, 6H), 1.47-1.37 (m, 2H). ¹³C NMR (101 MHz, Methanol-d₄) δ=174.26, 173.11, 172.56, 161.13, 146.47, 144.26, 143.62, 142.38, 141.73, 141.20, 131.10, 131.01, 129.92, 126.46, 126.27, 123.40, 123.28, 123.25, 123.12, 113.68, 112.12, 112.08, 101.80, 101.31, 54.50, 50.29, 50.24, 49.85, 44.91, 41.78, 34.97, 34.77, 34.38, 31.71, 28.68, 28.51, 28.35, 28.00, 27.39, 25.71, 25.35, 25.29, 22.08. HRMS m/z (ESI+) C₅₁H₆₂Cl₂N₃O₄⁺ calculated [M+] 850.4112, found 850.41263.

Solid phase synthesis of Octreotide amide-GABA-NH2 (OctA-GABA-NH2): The synthesis of the cyclic peptide octreotide was done according to a previously described procedure [Redko, B. et al., Biopolymers 2015, 104, 743-752; Gilad, Y. et al., Bioorg. Med. Chem. 2016, 24, 294-303; Redko, B. et al., Oncotarget 2017, 8, 757-768; and Gellerman, G et al., J. Pept. Res. 2001, 57, 277-291]. Rink Amide resin (0.65 mmol/g) was placed in a sintered glass bottom and swelled in NMP by agitation overnight. The Fmoc group was removed from the resin by treatment with 20% piperidine/NMP (2×15 minutes). The completion of the removing of Fmoc was monitored by ninhydrin test (blue). After washing the resin with NMP (7×2 min), a mixture of Fmoc-Thr(Trt)-OH (2 eq.), DIPEA (8 eq.) and PyBOP (2 eq) in NMP was added. The reaction was carried out for 1.5 h at room temperature. After coupling, the peptidyl resin was washed with NMP (5×2 min). The completion of the reaction was monitored by ninhydrin test (yellow). Then a linear SPPS was applied using standard Fmoc procedures introducing the amino acids in the following order: Fmoc-Cys(Acm)-OH, Fmoc-Thr(Trt)-OH, Fmoc-Lys(Boc)-OH, Fmoc-DTrp(Boc)-OH, Fmoc-Phe-OH, Fmoc-Cys(Acm)-OH, Fmoc-DPhe-OH, Fmoc-GABA-OH. All the couplings were performed in NMP with 2-fold excess of amino acid, DIPEA (8 eq) and PyBop (2 eq.) for activation. Each coupling cycle was conducted for 1.5 h. The completion of each coupling reaction and Fmoc removal were monitored by the ninhydrin test. After the coupling of the last amino acid, the sequence was cyclized by 12 (10 eq.) in DMF:H₂O (4:1) for 2 h, washed (DMF:H₂O (4:1) 5×2 min, DCM 5×2 min, chloroform 5×2 min, 2% ascorbic acid in DMF, DMF 5×2 min) and N-terminus Fmoc was deprotected using regular procedure yielding the cyclic peptidyl residue (OctA-GABA-NH2) on Rink Amide resin ready for next conjugation.

Synthesis of OctA-GABA-IRD-CLB (5-CLB): A mixture of RD (40 mg, 72 μmol, 1.2 eq.), HATU (35 mg, 90 μmol, 1.5 eq.) and DIPEA (50 μl, 300 μmol, 5 eq) in NMP was stirred for preactivation for 2 min and consequently added to the OctA-GABA-NH2 (immobilized on solid phase) (200 mg, 60 μmol, 1 eq). The coupling reaction was carried out for 1.5 h at room temperature. After coupling (ninhydrin test—yellow), the peptidyl resin was washed with 4 ml NMP (5×2 min) and DCE 4 ml (5×2 min). Thereafter, CLB (37 mg, 120 μmol, 2 eq) was activated by treatment with BTC (26 mg, 90 μmol, 1.5 eq.) and collidine (45 μl, 300 μmol, 6 eq.) in 4 ml DCE for 1 min and added to the peptidyl resin. The reaction was carried out for 2 h. After coupling, the peptidyl resin was washed with DCE 4 ml (5×2 min).

Cleavage procedure for Rink Amide resin: 5-CLB on the peptidyl-resin was treated with the cold TFA without scavengers for 1.5 h. Noteworthy, addition of the scavengers like TIS or EDT lead to the decomposition of the product. The solvent was removed under a gentle flow of N₂, cold Et₂O added and the crude product was precipitated from Et₂O. The obtained crude 5-CLB was purified by preparative HPLC, pure fractions were lyophilized yielding 6.3 mg (5.4% overall yield calculated by peptide loading) of pure 5-CLB. HRMS m/z (ESI+) C₁₀₄H₁₃₂Cl₂N₁₅O₁₄S₂⁺ calculated: 1949.8925, found 650.96985 ((C₁₀₄H₁₃₂Cl₂N₁₅O₁₄S₂⁺+2H⁺)/3).

### Absorption and Fluorescence:

Absorption spectra were recorded on a Jasco V-730 UV-Vis spectrophotometer and the fluorescence spectra were measured on Edinburgh FS5 spectrofluorometer. The absorption and fluorescence spectra and the quantum yields (ΦF) were measured in a 1-cm quartz cell at ˜0.5 μM dye concentrations in PB 10 mmol buffer pH 7.4 at 25° C. and cell medium pH 7.4. Excitation wavelength was 720 nm and 532 nm. The recorded fluorescence spectra were corrected using the wavelength-dependent instrument sensitivity coefficients. Absorption and emission maxima were measured with accuracy of ±0.3 nm and ±0.5 nm, respectively, and rounded. For the determination of the quantum yields, the integrated relative intensities were measured versus ICG™ in methanol as the reference (Φ_{F}=0.043) and Cy3™ in PBS (Φ_{F}=0.043). The absorbance values measured for the samples and the reference at the excitation wavelength were 0.04-0.06 measured in a 1-cm cell. The absolute quantum yields were calculated according to Equation 1.

Φ_{F}=Φₛₜ×(F/Fₛₜ)×(Aₛₜ/A)(n²/n²st) Equation (1)

The quantum yield of each sample was independently measured three times and the average value was taken. The reproducibility was within 5%.

### Cleavage Rates for IRD-CLB and 5-CLB

A stock solution of IRD-CLB and 5-CLB in acetonitrile was added to PB 10 mmol buffer pH 7.4 at 25° C. and cell medium pH 7.4, which both contained 20% of acetonitrile. The absorbance of the resulted solutions at the maximum was 0.10±0.01, when measured in a 1-cm standard quartz cell. The solutions were incubated at 37° C. during 24 hours. In certain periods of time fluorescence spectrum was measured.

### Cytotoxicity

The cytotoxicity of the peptide-drug conjugates was determined by measuring the mitochondrial enzyme activity, using a commercial XTT assay kit. Panc-1 cell line was cultured in RPMI medium supplemented with 10% heat-inactivated Fetal Bo-vine Serum (FBS), 2 mM glutamine, 1% penicillin and strep-tomycin and cultured at 37° C. in a humidified incubator with 6% CO2. Cells cultures were initiated in microplate wells at a concentration of 2-4×10⁴ and the cells were allowed to adhere for 24 hours. Thereafter, the cells were washed with PBS buffer twice before fresh PBS buffer containing 5% DMSO and different concentrations of the 5-CLB, CLB and OCT-Amide were added and the cells were incubated for an additional 10 minutes. The buffer was removed; all the wells were washed with PBS and cultured for 24 hours in fresh medium without drug substances. The cells were washed again and given a fresh medium containing the XTT reagent and incubated for 2 hours. Absorbance in the wells were measured with a TECAN Infinite M200 ELISA reader at both 480 and 680 nm—the latter being the background absorbance. The difference between these measurements was used for calculating the % of Growth Inhibition (GI) in test wells compared to the cells that were exposed only to the medium with 5% DMSO. All the tests were done in triplicate.

FIG. 11 presents a comparative plot, showing inhibition of the PANC-1 growth by 5-CLB, CLB and OctA, wherein at the end of 20 minutes incubation period and subsequent washing, cell growth was assessed using the XTT assay at 24 hours (the inhibition for each concentration point is represented by the mean±standard error for each independent experiment conducted in triplicate).

### Specificity

The specificity of 5-CLB towards PANC-1 cell line was supported also by a competitive binding of 5-CLB and commercially available octreotide to the PANC-1 receptors. In a series of experiments, PANC-1 cell line was incubated for 10 min in PBS buffer contained 5% DMSO with a constant concentration of 5-CLB (10 μM) and variable concentration of octreotide (×1, ×3 and ×10 molar excess) and washed out. Then the fluorescence images were taken in the NIR channel in 60 minutes after the incubation.

FIG. 12 presents a comparative plot showing a decrease of the normalized fluorescence intensity of PANC-1 after incubation with 5-CLB (10 μM) and Oct at different [Oct]/[5-CLB] ratios at 60 min after incubation, wherein the fluorescence intensity for each concentration point was measured in the NIR channel and represented by the mean±standard error for three independent experiments.

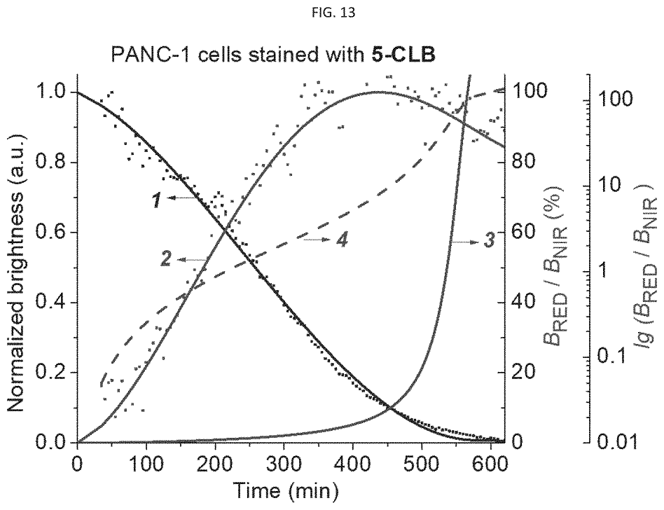

The specificity of 5-CLB towards SSTRs was assessed by testing whether free octreotide (Oct) can competitively inhibit delivery of 5-CLB to PANC-1. PANC-1 were incubated for 10 minutes at 37° C. in PB with 5-CLB (10 μM) and increasing concentrations of Oct (xl, x3 and x10 molar excess) and both compounds were washed out. Fluorescence images were taken at 60 min after the incubation and fluorescence intensities remaining in the cells were measured. Fluorescence of 5-CLB accumulated in the cells decreased proportionally to the Oct concentration, indicating that the two compounds compete for the same binding sites, i.e. SSTRs. At the next step, the ratiometric fluorescence monitoring of CLB release from the 5-CLB conjugate delivered into the PANC-1, has been conducted. The cells were incubated with 5-CLB for 10 min to allow accumulation of the compound, and fluorescence images were taken over time in two channels. The NIR channel enabled measurements of the signal from IRD dye conjugated with CLB (5-CLB) while the Red channel detected the signal from the RD fluorophore formed upon the CLB release. The representative time-dependent fluorescence values obtained in the red and NIR channels and an overlay of these two channels with transmitted light values are shown in FIG. 13.

FIG. 13 presents a comparative plot showing the CLB cleavage profiles obtained by fluorescence imaging of PANC-1 cell line stained with 5-CLB, wherein the plot marked by “1” shows the decrease of the BNIR, the plot marked as “2” shows an increase of the BRed, the plot marked as “3” shows ratiometric curve (BRed/BNIR), and, the plot marked as “4” shows ratiometric curve in logarithmic scale [lg(BRed/BNIR)].

As can be seen in FIG. 13, the fluorescence intensity of IRD (in NIR channel) decreases with time while the RD signal (in Red channel) increases; and simultaneously. The quantitation of the time-dependent changes in cell brightness in the NIR and Red channels (B_{NIR} and B_{Red}) is also shown in FIG. 13 (curves 1 and 2). Indeed, the BNIR brightness decreases while the BRed increases, signaling the CLB cleavage rate with half-life of about τ_{1/2}˜200 min. Corresponding ratiometric curves obtained from these two signals (B_{Red}/B_{NIR}) are presented in FIG. 13 (curve 3). As compared to the intensity based profiles, this curve enables concentration-independent quantitative measurements of the drug release degree in cells.

In summary, the long-wavelength fluorescent cyanine dye IRD, provided herein according to some embodiments of the present invention, containing triggering hydroxyl group in the polymethine chain and single carboxyl functionality, was synthesized and tested successfully. By means of a biodegradable ester bond, the hydroxyl function of the dye was bound to the carboxyl group of anticancer drug chlorambucil (CLB). In addition, the carboxyl functionality of IRD was coupled to cancer-specific peptide octreotide amide (OctA) through a non-degradable GABA linker to form the 5-CLB theranostic conjugate. The synthetic approach to this conjugate is elaborated hereinabove.

The spectroscopic study shows that IRD dye bound to CLB exhibits absorption and emission in the NIR spectral region while upon the drug release the fluorescence turns red. Due to both CLB-bound and unbound forms of IRD dye are fluorescent and the emissions of these forms lie in different spectral range, the IRD dye is suitable for the ratiometric fluorescence measurements. In addition, the long-wavelength emission of this dye is beneficial for sensing applications in body. The environment-mediated CLB cleavage rates were investigated in phosphate buffer and esterase containing cell medium; and the half-lives of drug release for IRD-CLB were found to be about 25 hours in PB and 1.3 hours in CM, and about 2.4 hours, and about 2.0 hours, respectively, for 5-CLB. Thus, conjugation of OctA with IRD-CLB to form 5-CLB noticeably accelerates the CLB release in PB but slows down it in CM.

Furthermore, the OctA-guided targeted drug delivery by 5-CLB conjugate to human pancreatic cancer cell line PANC-1 has been demonstrated hereinabove. A comparative XTT cell survival assay carried out for 5-CLB conjugate vs. CLB and OctA taken separately evidences that OctA peptide dramatically enhances delivery of the drug in PANC-1. The specificity of the OctA-guided targeting was confirmed also by a fluorescently monitored competitive binding of 5-CLB conjugate to PANC-1 vs. free Oct.

The environment mediated CLB release from 5-CLB conjugate in PANC-1 was monitored using fluorescence microscopy. The corresponding fluorescence intensity-based profiles of CLB release show a time-dependent decrease of the 5-CLB concentration (decrease of the NIR emission) and increase of the free CLB concentration (increase of the red emission).

Finally, in the example of PANC-1, the 5-CLB conjugate equipped with IRD dye was proved to enable the real time ratiometric fluorescence TDD monitoring. As compared to the intensity based profiles, the ratiometric curve provides concentration-independent quantitative measurement of the drug release degree in cells. By taking the ratio between the red and NIR intensities over time, the concentration of the released CLB drug can be directly correlated to these kinetics curves. Using the obtained profiles the half-life of the CLB release in PANC-1 cell line was estimated to be about 200 min.

Ultimately, in the in vitro imaging modality the developed theranostic conjugate 5-CLB was utilized successfully to prove the principle of the ratiometric fluorescence monitoring of carrier-targeted drug delivery and quantitative determination of the drug release degree in target tissues.

### Example 3

### Highly Bright, Switchable Reporters and Conjugates, with an Increased Fluorescence Intensity Dynamic Range

### Switchable NIR Reporters and Conjugates “Drug-Switchable Reporter”:

A series of switchable reporters are synthesized, containing reactive groups for conjugation and hydrophilic groups for adjusting the hydrophobic-hydrophilic properties of the theranostic conjugates. To increase the fluorescence intensity dynamic range of these conjugates, one evaluates different classes of dyes and adjusts substituents and cleavable linkers. Naphthofluorescein is suggested as the switchable reporter, as it is a longer-wavelength analog of the recently developed fluorescein-based switchable dye, as well as a series of cyanine and styryl dyes that contain trigger moieties, such as phenolic (“1” in Scheme 6 below), 2,3-dihydro-1H-xanthen-6-ol (“2” in Scheme 6 below), 7-hydroxynaphthalen-2(1H)-one (“3” in Scheme 6 below), and 6-hydroxyquinolin-3(4H)-one (“4” in Scheme 6 below).

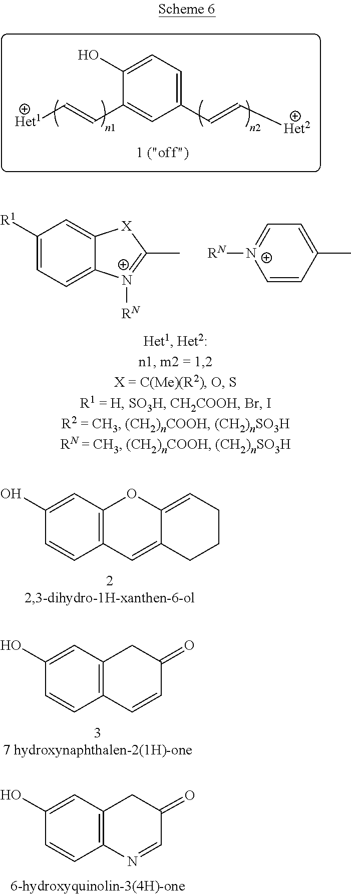

The general structure of phenolic cyanine dyes (“1” in Scheme 6) is shown in Scheme 6. To reduce the spectral overlap between the two reporters, one may adjust the spectral range by varying the heterocyclic terminal end-groups (Het¹, Het²), e.g., to indoleninium, benzoxazolium, benzothiazolium, or pyridinium, and/or by changing the length of polymethine chain (n1, n2). Heterocycles (Het¹, Het²) and the chain length (n1, n2) can be either identical (Het¹=Het²) or different (Het¹≠Het²). Indoleninium-based cyanines, compared with other cyanines, typically exhibit better stability and higher fluorescence quantum yields in conjugates. The introduction of benzoxazolium and benzothiazolium moieties induces a ˜20 nm blue and red spectral shifts, respectively, while increasing the length of the conjugated chain (n1, n2=2) and is known to shift the absorption and fluorescence maxima to the longer-wavelength range (by ˜70-100 nm).

Phenolic indoleninium cyanines (n1, n2=1) are synthesized in a drug-conjugated “off” form, as shown in Scheme 7). Cyanine dyes containing other heterocycles (Het¹, Het²) can be synthesized using the same method.

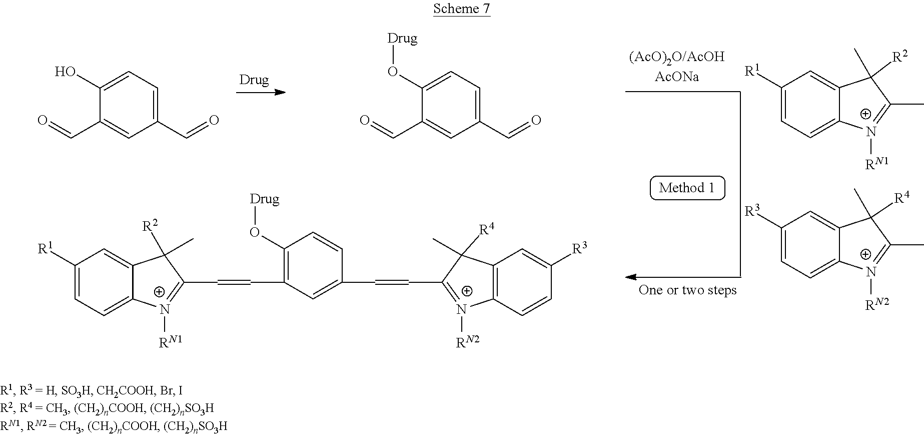

Alternatively, a one-step conjugation of the drug to the dye molecules is also contemplated (see, Scheme 8 below).

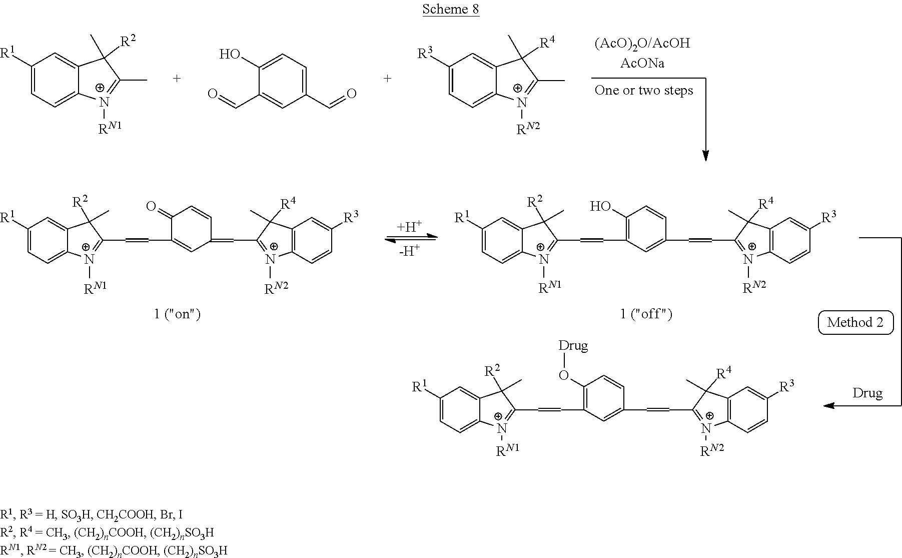

Also contemplated are cyanines based on 2,3-dihydro-1H-xanthen-6-ol (“2” in Scheme 6), 7-hydroxynaphthalen-2(1H)-one (“3” in Scheme 6), and 6-hydroxyquinolin-3(4H)-one (“4” in Scheme 6) according to Scheme 9 and Scheme 10. The synthesis of dyes 2 and 3 requires the same starting indolenines as the above-mentioned phenolic cyanines, which simplifies implementation.

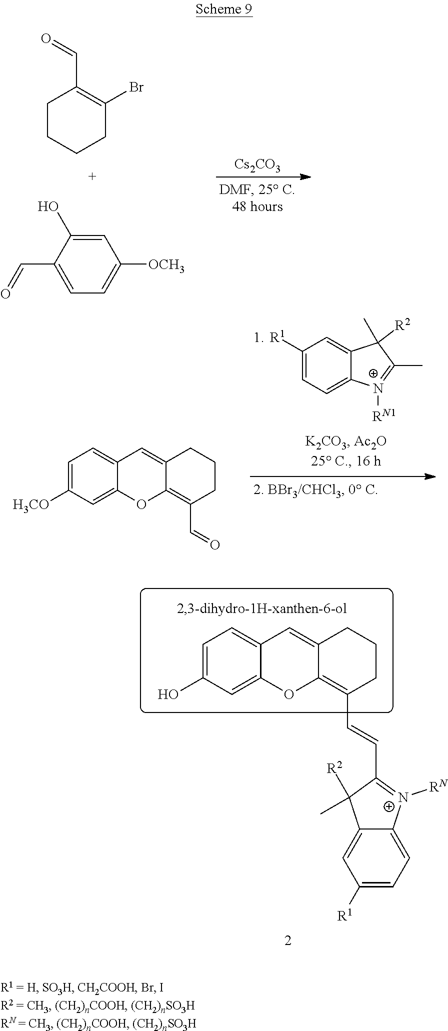

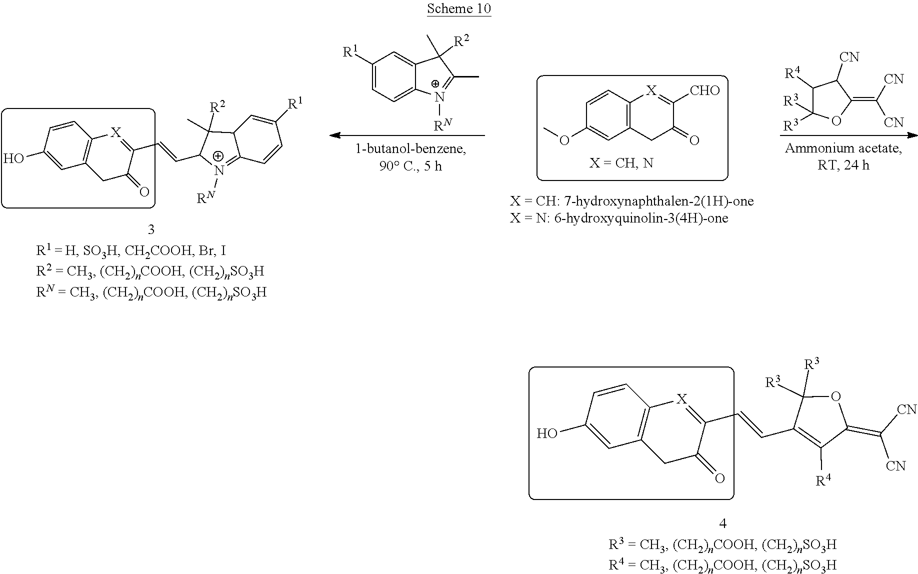

To attach the reporters to the drugs, one can use cleavable linkers as shown in Scheme 7 and Scheme 8). Preliminary results show that the structures of the drug and dye can noticeably affect the cleavage rate of the linker. Therefore, one can investigate a series of cleavable linkers, in particular, esters, carbamates, and disulfides. One will select the linkers to achieve cleavage rates that are reasonable for theranostic applications (in general, a cleavage half-life in the order of 0.5-2 hours).

### Spectroscopic Characterization:

Spectroscopic characterization of the switchable reporters and conjugates, including drug-release profiles in various media includes spectral, photophysical, and photochemical characterization for recognizing the most promising candidates for producing the desired theranostic conjugates. One can measure the absorption and fluorescence spectra, extinction coefficients, fluorescence quantum yields, and brightness of the dyes, and estimate the chemical and photochemical stability of these dyes and the drug-release profiles in various media. The major criteria for these switchable dyes will be: (a) absorption and fluorescence of the “on” form in the red-NIR region; (b) a high dynamic range (at least ×20) of brightness change from the “on” to the “off” form (the fluorescence of the “on” form should preferably be non-detectable); (c) high brightness (B>50,000 M⁻¹ cm⁻¹); and (d) chemical stability in conjugation reactions and photochemical stability sufficient for monitoring (τ_{1/2} longer than ˜12 hours).

### Example 4

### Synthesize of Dual-Dye Theranostic Conjugates for Quantitative Monitoring of TDD

### Synthesis of the Reference NIR Reporters:

For reference reporters, one can use known penthamethine and hepthamethine cyanine fluorophores that are insensitive to the environment. One can select these reporters to decrease as much as possible the overlap between their spectral bands and those of the switchable dyes. The reference reporters may contain reactive groups for binding to the theranostic conjugate by non-cleavable linkers and hydrophilic groups to adjust their solubility and penetration ability through cell membranes. Examples of these reporters are presented in Scheme 11 below, showing examples of reference reporters. n=1, 2; m1, m2, k1, k2=3-5; R^{X}—reactive group selected from NHS ester, maleimide, hydroxy, amino, and click-chemistry groups.

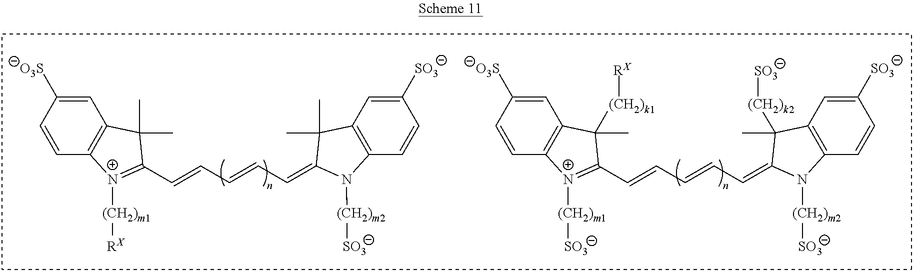

Synthesis of the resin-loaded targeting peptides: For the targeting group, one can use a peptide (rather than an antibody), and utilize a short, cyclic targeting peptide, the Cilengitide derivative c(RGDfK), which is a selective, high-affinity ligand to the aᵥb₃ Integrin that is over-expressed in cancerous cells. It has been proven to be an effective carrier for TDD because it undergoes rapid internalization, quick circulatory clearance, and good tumor tissue-penetrating capability. In addition, this peptide is also metabolically stable and is easily synthesizable in cost effective solid-phase chemistry. It is noted that c(RGDfK) possesses an amine functionality, which allows its conjugation.

### Synthesis of Novel, Dual-Dye Theranostic Conjugates:

Synthesis of novel, dual-dye theranostic conjugates, of the sort “Drug-Switchable reporter-Reference reporter-Peptide” is shown in Scheme 12, which presents one of the possible solid-phase peptide approaches to the dual-dye theranostic conjugates.

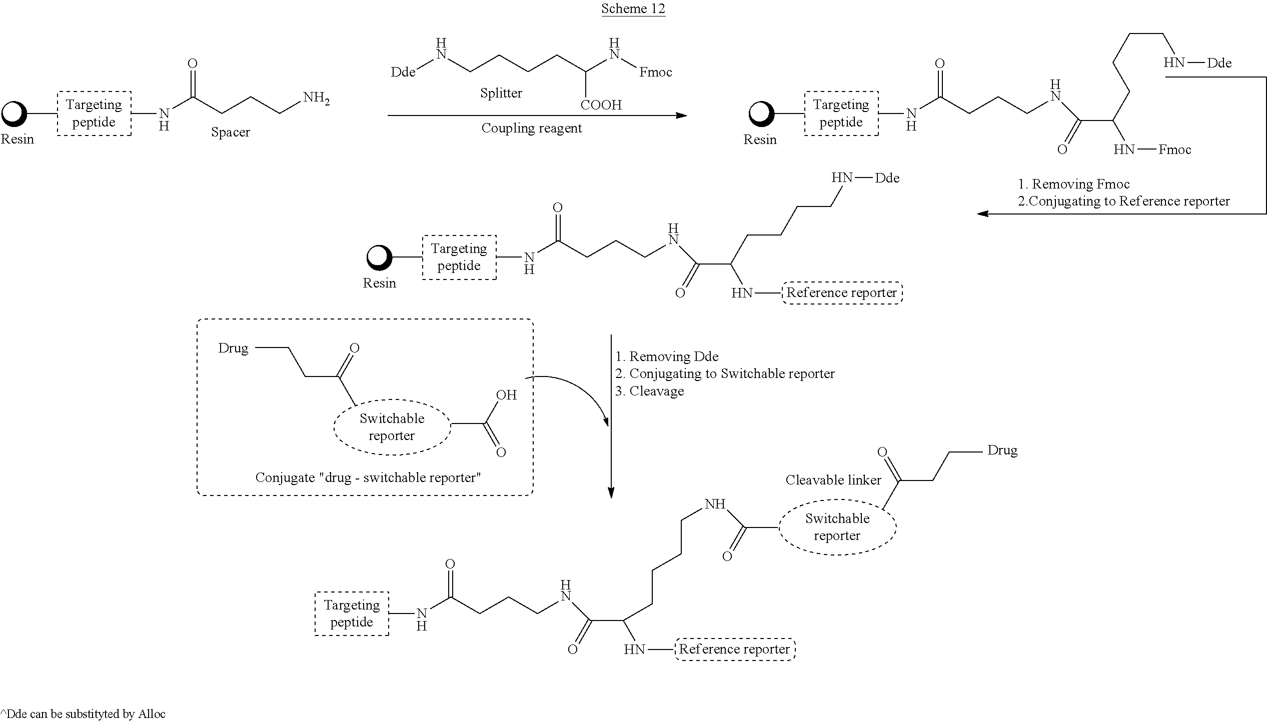

Scheme 13 presents examples of syntheses of “Drug-Switchable reporter” conjugates.

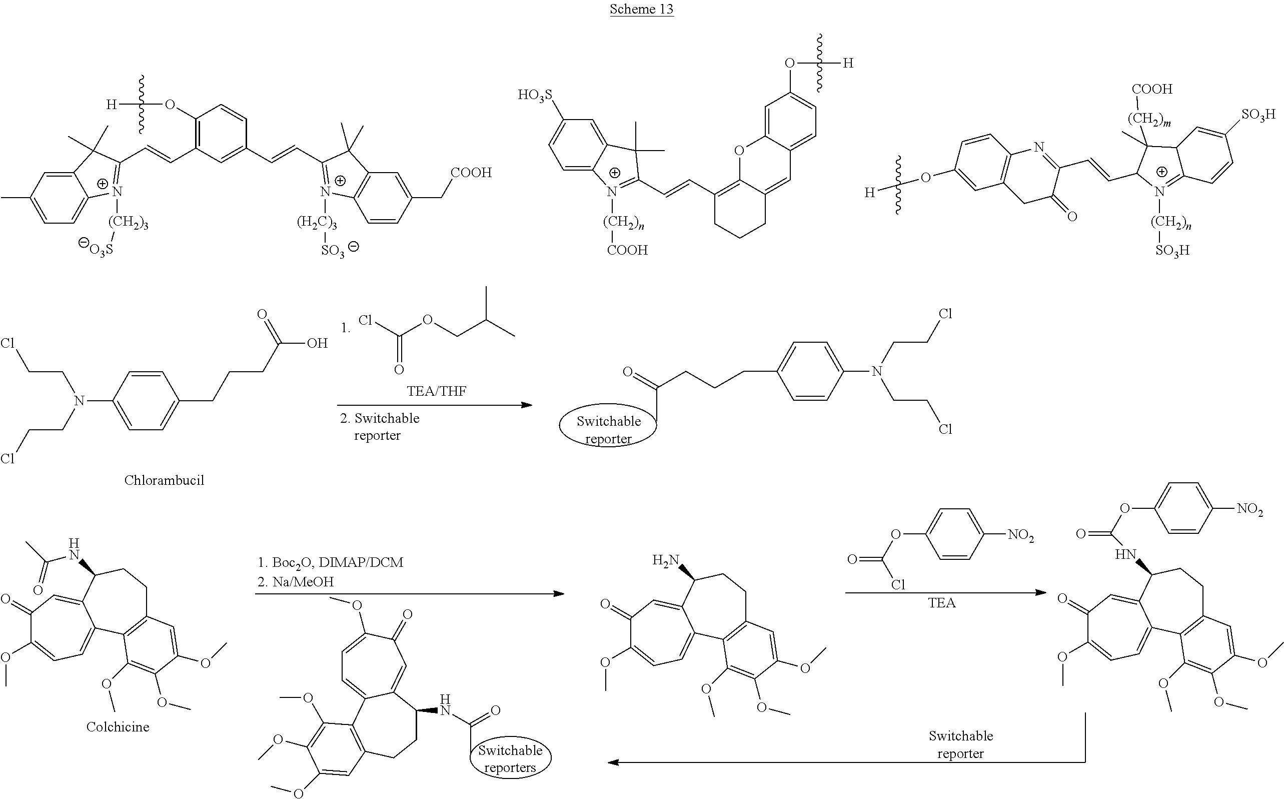

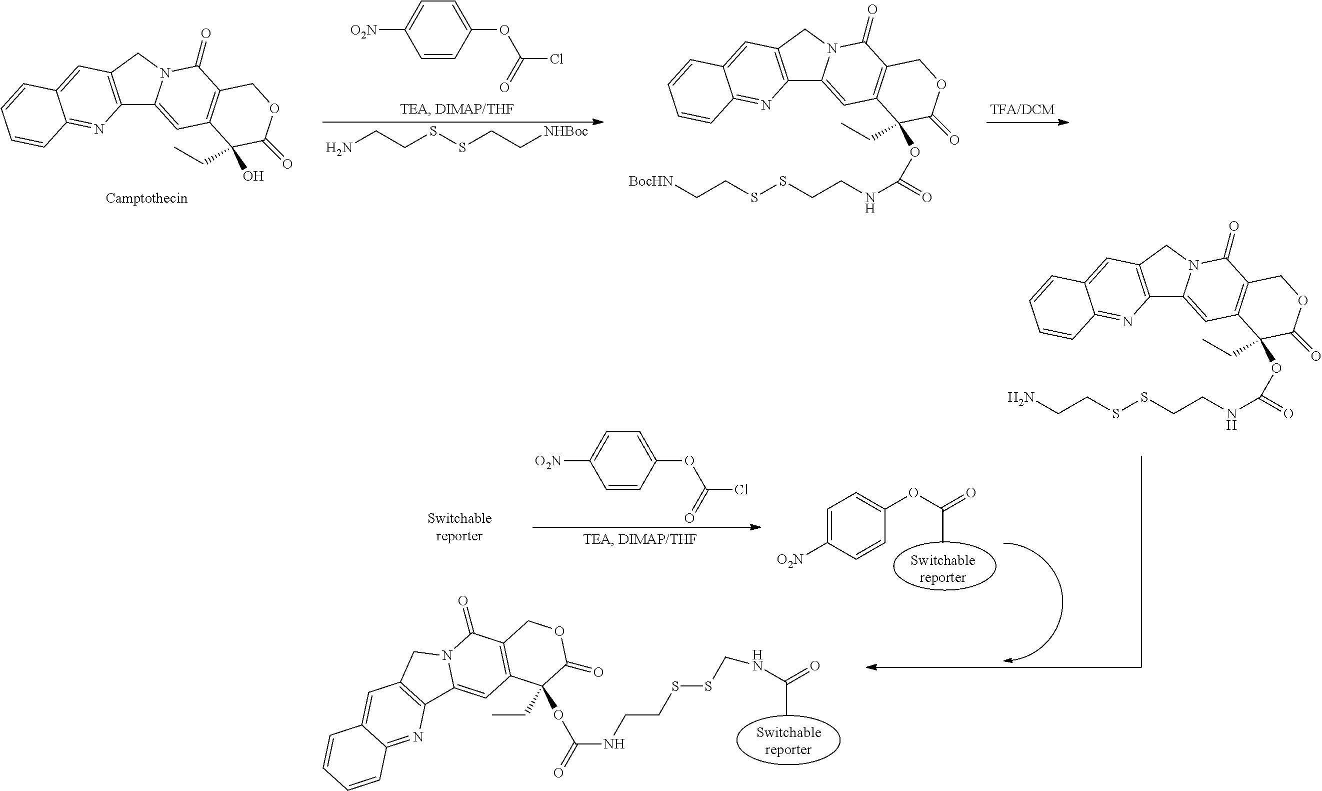

The performance of the above dual-dye theranostic conjugates ca be compared to that of conventional, single-dye conjugates. These conjugates have the same structure as the dual-dye conjugates shown in Scheme 12 and Scheme 13 but without the reference reporter; they comprise the same switchable dyes, drugs, targeting peptide, and linkers as the dual-dye conjugates, thus ensuring an adequate comparison, and the compounds can be synthesized by known methods.

### Spectral Characterization of the Single-Dye and Dual-Dye Conjugates:

Spectral characterization of the single-dye and dual-dye conjugates, including their drug-release profiles in various media is effected by standard spectroscopic methodologies. One can characterize the absorption and fluorescence spectra, brightness in the “on” and “off” forms, and dynamic range of fluorescence intensity change upon drug release. One can also determine the drug cleavage rates in different environments, in particular, at low and high pH and in cell media, and obtain the drug-release profiles. If the cleavage rates are too short (the half-life shorter than 10 min) or too long (longer than 4 hours), one can adjust the cleavable linkers accordingly. The profiles obtained for the dual-dye conjugates may be compared to those obtained for the single-dye conjugates to confirm that the reference reporter indeed has a negligible effect on the drug cleavage rate and efficacy; otherwise, one can calibrate the profiles obtained for the switchable reporter of the dual-dye conjugates to fit those of the single-reporter conjugates.

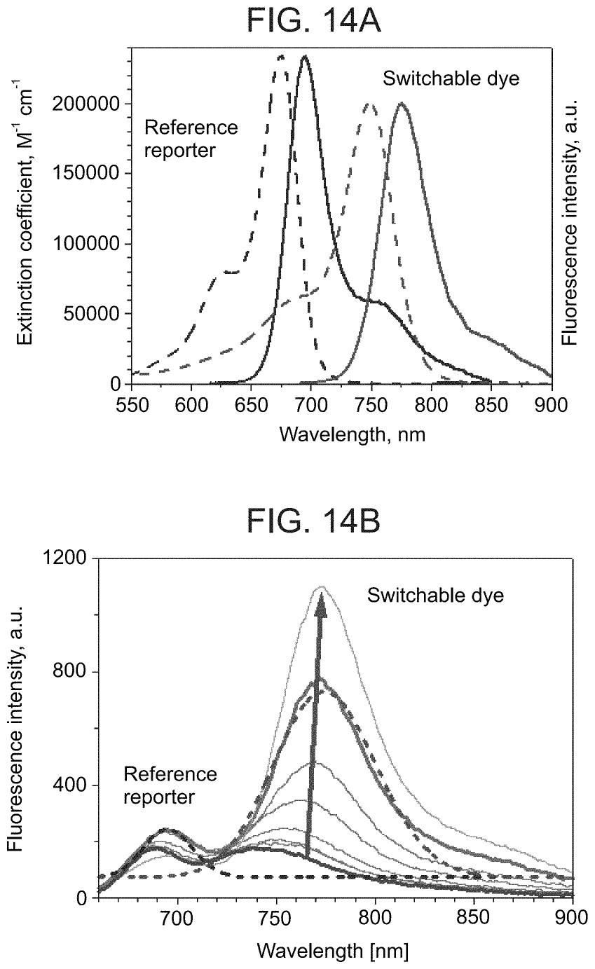

The experimental spectral curve is mathematically separate into the individual bands of the reference reporter and the switchable reporter (see, FIGS. 14A-B) and estimate the fluorescence resonance energy transfer (FRET) between them. To quantify the concentrations of the entire conjugate and released drug, one can use the fluorescence signals of the reporters and the calibration curves obtained by liquid chromatography-mass spectrometry (LCMS).

FIGS. 14A-B present absorption (dashed line) and fluorescence (solid line) spectra of representative reference reporter and switchable dye (FIG. 14A), and the anticipated experimental fluorescence spectra affected by FRET (FIG. 14B).

As can be seen in FIGS. 14A-B, the fluorescence signal of the switchable dye increases following drug release. The signal of the reference reporter may decrease due to the FRET with the switchable reporter. Data will be processed by mathematically separating the experimental spectral curve (FIG. 14B) into the individual signals (dotted lines) of the reference reporter and switchable dye.

### Quantification of Drug-Delivery Efficacy:

Based on the ratios between the fluorescence signals of the switchable dye and of the reference reporter, one can calculate R_{eff} at different time points following drug release. Such quantification is possible only with the presently provided dual-dye conjugates, according to embodiments of the present invention.

### Example 5

### Synthesis and Use of Activatable Sensitizers and Dual-Dye PDT Conjugates

Synthesis of activatable NIR sensitizers with an increased dynamic range:

One can use the methods described above for the reporters, while employing heavy halogen atoms to increase the efficacy of the sensitizer. It has been found that introducing heavy halogen atoms, such as bromine or iodine, in cyanine dyes increases both the brightness and the ability of these dyes to generate reactive cytotoxic species, a feature for both imaging and PDT. Based on this finding, one can design a series of sensitizers. This approach, which utilizes the same fluorophores to construct both the reporters and the sensitizers, considerably simplifies implementation of some embodiments of the present invention.

Scheme 14 below presents examples of switchable sensitizers, according to some embodiments of the present invention, and of syntheses of conjugates “Activatable Sensitizer-Reference Reporter-Targeting Peptide” for PDT.

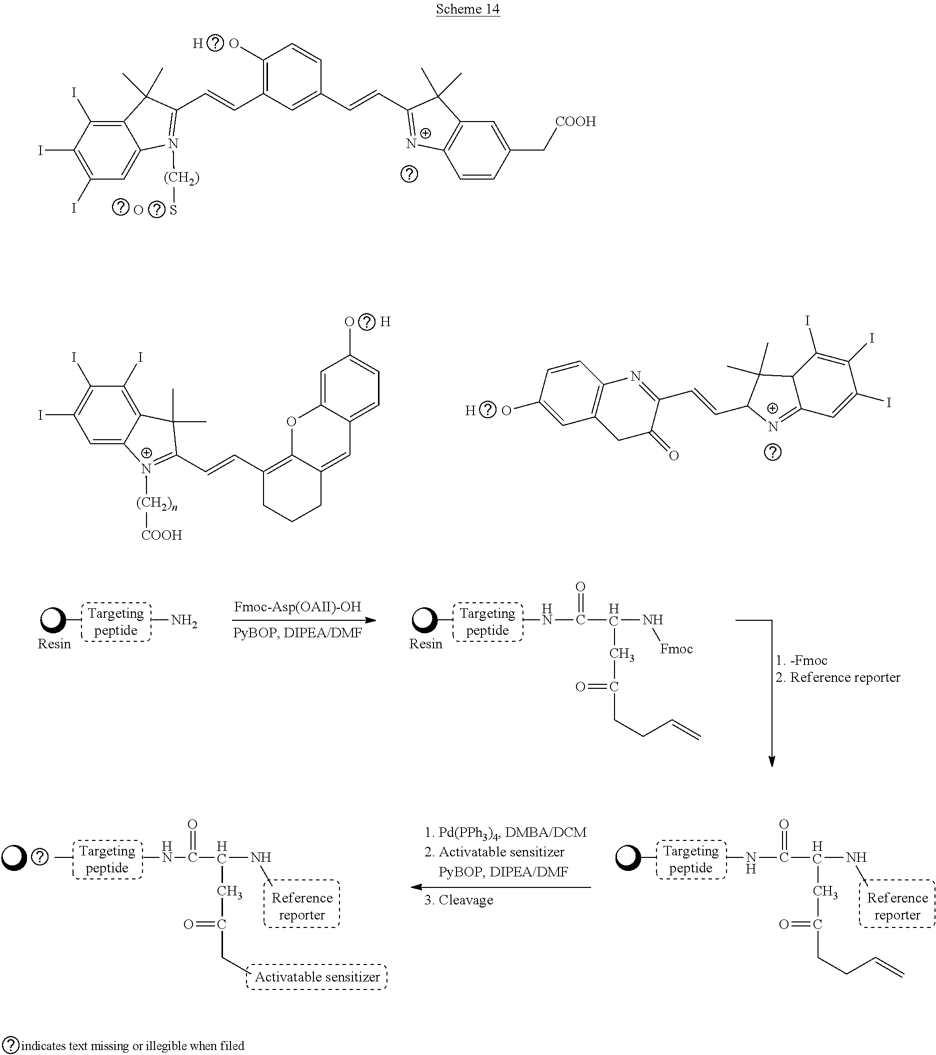

Spectral Characterization of Activatable NIR Sensitizers and Investigation of their PDT Efficacy on Cells:

One can characterize the sensitizers similarly to reporters, and also measure their dark cytotoxicity and sensitizing efficacy in cells, using established methods.

### Example 6

### Synthesis and Use of Dual-Dye Theranostic Conjugate Aza-FLU-Cy5-Lys-COOH

Scheme 15 below presents a non-limiting example of a dual-fluorophore conjugate, according to some embodiments of the present invention, designed to deliver azatoxin (Aza), and referred to herein as Aza-FLU-Cy5-Lys-COOH. As can be seen in Scheme 15, the conjugate comprises, circles from left to right, encircle a reference signal moiety (FLU), a spacer (Lys) for attachment of a targeting moiety; a switchable signal moiety (Cy5), a cleavable linker (FLU); and a bioactive agent (Aza).

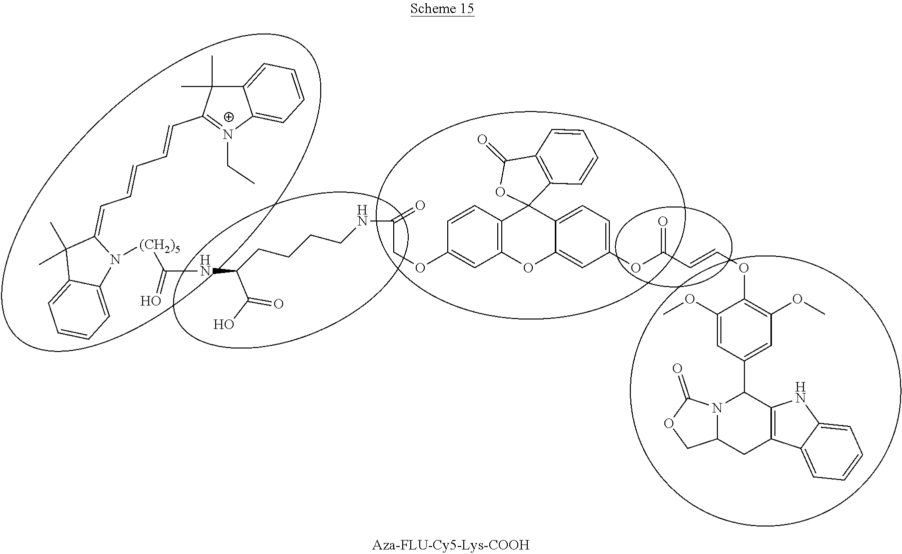

### Synthesis of Aza-FLU-tBu:

Synthesis of (E)-3′-(2-(tert-butoxy)-2-oxoethoxy)-3-oxo-3H-Spiro [isobenzofuran-1,9′-xanthen]-6′-yl 3-(2,6-dimethoxy-4-(3-oxo-1,3,5,6,11,11a-hexahydrooxazolo[3′,4′:1,6]pyrido[3,4-b]indol-5-yl)phenoxy)acrylate (Aza-FLU-tBu) was carried out by dissolving azatoxin (see, “2” in Scheme 16 below), (200 mg, 0.53 mmol) and DABCO (6 mg, 0.053 mmol) in THF (10 mL). The solution was stirred for 30 minutes at room temperature. Thereafter, compound 1 (see, Scheme 16 below), (264.2 mg, 0.53 mmol) was dissolved in 5 mL of THF and added dropwise, followed by overnight stirring of the reaction mixture. After completion of reaction, solvent was evaporated and compound Aza-FLU-tBu, (428.5 mg, 0.49 mmol) was isolated as red oil in 92% yield after purification on silica.

¹H NMR (400 MHz, CHLOROFORM-d) δ ppm: 8.14 (s, 1H), 8.02 (d, J=7.46 Hz, 1H), 7.83 (d, J=12.23 Hz, 1H), 7.59-7.72 (m, 2H), 7.55 (d, J=7.70 Hz, 1H), 7.35 (d, J=7.95 Hz, 1H), 7.13-7.26 (m, 3H), 7.09 (t, J=1.90 Hz, 1H), 6.58-6.83 (m, 7H), 6.06 (s, 1H), 5.45 (d, J=12.23 Hz, 1H), 4.54 (s, 2H) 4.61 (t, J=8.31 Hz, 1H), 4.14-4.31 (m, 2H), 3.76 (s, 6H), 3.20 (dd, J=14.98, 4.58 Hz, 1H), 2.85 (ddd, J=14.95, 10.55, 1.41 Hz, 1H), 1.50 (s, 9H).

### Synthesis of Aza-FLU—COOH:

Synthesis of (E)-2-((3′-((3-(2,6-dimethoxy-4-(3-oxo-1,3,5,6,11,11a-hexahydrooxazolo[3′,4′:1,6]pyrido[3,4-b] indol-5-yl)phenoxy)acryloyl)oxy)-3-oxo-3H-spiro [isobenzofuran-1,9′-xanthen]-6′-yl) oxy) acetic acid (Aza-FLU—COOH) was carried out by treating Aza-FLU-tBu (100 mg, 0.11 mmol) for 1 hour in a cold mixture of TFA/DCM (1:1). Thereafter the solvent was evaporated under N₂ stream, and the residue was purified on preparative HPLC giving Aza-FLU—COOH as a red powder (64.5 mg, 0.078 mmol) in 71.3% yield after lyophilisation.

¹H NMR (400 MHz, CDCl₃) δ ppm: 7.99-8.05 (m, 1H), 7.97 (s, 1H), 7.82 (dd, J=12.23, 0.73 Hz, 1H), 7.59-7.71 (m, 2H), 7.55 (d, J=7.70 Hz, 1H), 7.31-7.37 (m, 1H), 7.12-7.25 (m, 3H), 7.08 (t, J=2.14 Hz, 1H), 6.60-6.84 (m, 7H), 6.07 (s, 1H), 5.45 (dd, J=12.23, 0.86 Hz, 1H), 4.70 (s, 2H), 4.61 (t, J=8.38 Hz, 1H), 4.15-4.34 (m, 2H), 3.70-3.81 (m, 6H), 3.21 (dd, J=14.98, 4.58 Hz, 1H), 2.88 (ddd, J=15.01, 10.55, 1.47 Hz, 1H).

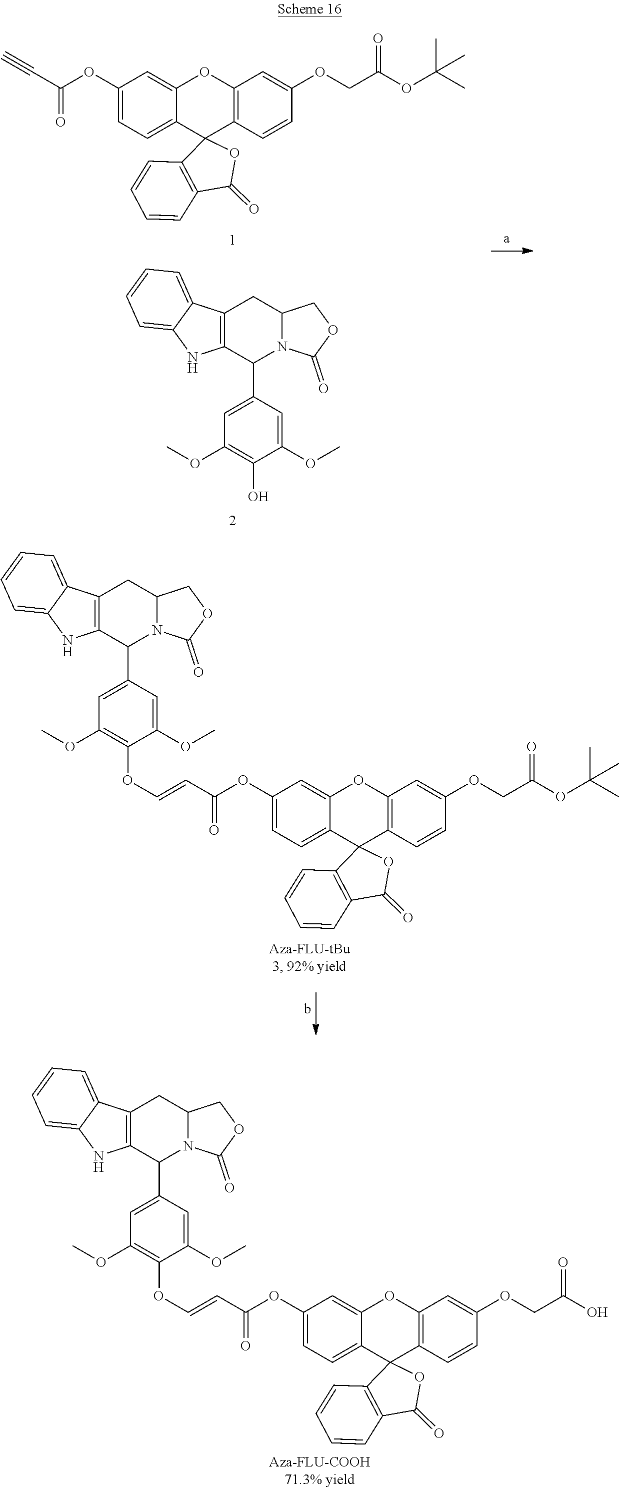 [SMILES: C#CC(=O)Oc1ccc2c(c1)Oc1cc(OCC(=O)OC(C)(C)C)ccc1C21OC(=O)c2ccccc21 ; COc1cc(C2c3[nH]c4ccccc4c3CC3COC(=O)N32)cc(OC)c1O ; COc1cc(C2c3[nH]c4ccccc4c3CC3COC(=O)N32)cc(OC)c1O/C=C/C(=O)Oc1ccc2c(c1)Oc1cc(OCC(=O)OC(C)(C)C)ccc1C21OC(=O)c2ccccc21 ; COc1cc(C2c3[nH]c4ccccc4c3CC3COC(=O)N32)cc(OC)c1O/C=C/C(=O)Oc1ccc2c(c1)Oc1cc(OCC(=O)O)ccc1C21OC(=O)c2ccccc21]

### Synthesis of Cy5-Lys:

2-clorotrytyl resin, (200 mg, 1.12 mmol/g) was placed in a reactor under nitrogen atmosphere followed by addition of Fmoc-Lys(MTT)-OH (100 mg, 0.16 mmol) and DIPEA (140 μL, 0.8 mmol) in dry DCM (20 mL). The reaction mixture was shake 1 hour at room temperature, and cupped with MeOH (2×15 min). The obtained Fmoc-protected peptidyl resin was shacked with 20% solution of piperidine in DMF (20 mL, 2×15 min) and washed with DMF (20 mL, 2×3 min) and DCM (2×3 min) and coupled with Cy5 (79.52 mg, 0.16 mmol) using HATU (60.8 mg, 0.16 mmol) as a coupling reagent and DIPEA (140 μL, 0.8 mmol). After completion of the reaction (monitored by LCMS) resin was cleaved by 2% TFA in DCM (40 min, room temperature). The solvent was evaporated under N₂ stream, and residue was purified on preparative HPLC and lyophilized to give Cy5-Lys (97 mg, 0.13 mmol, 82% yield) as a violet powder.

### Synthesis of Aza-FLU-Cy5-Lys-COOH:

Aza-FLU—COOH (64.5 mg, 0.078 mmol), HATU (29.6 mg, 0.078 mmol) and DIPEA (67.8 μL, 0.39 mmol) were dissolved in DMF (1 mL) and vortexed during 1 min. The obtained solution was added to the vial contained 57.7 mg (0.078 mmol) of Cy5-Lys and reaction mixture was shacked 1 hour (see, Scheme 17 below). After completion of the reaction, the mixture was purified on preparative HPLC and lyophilized to give Aza-FLU-Cy5-Lys-COOH (2.12 mg, 0.05 mmol, 64% yield) as a violet powder. HPLC: R_{f}=10.210 min (254 nm gradient of 5-100% AcCN-H₂O+0.1% FA). HRMS: Calc.: (M)⁺ 1429.6067, (M+H)²⁺=715.3070; Found: (M)⁺=1429.6089, (M+H)²+=715.3086.

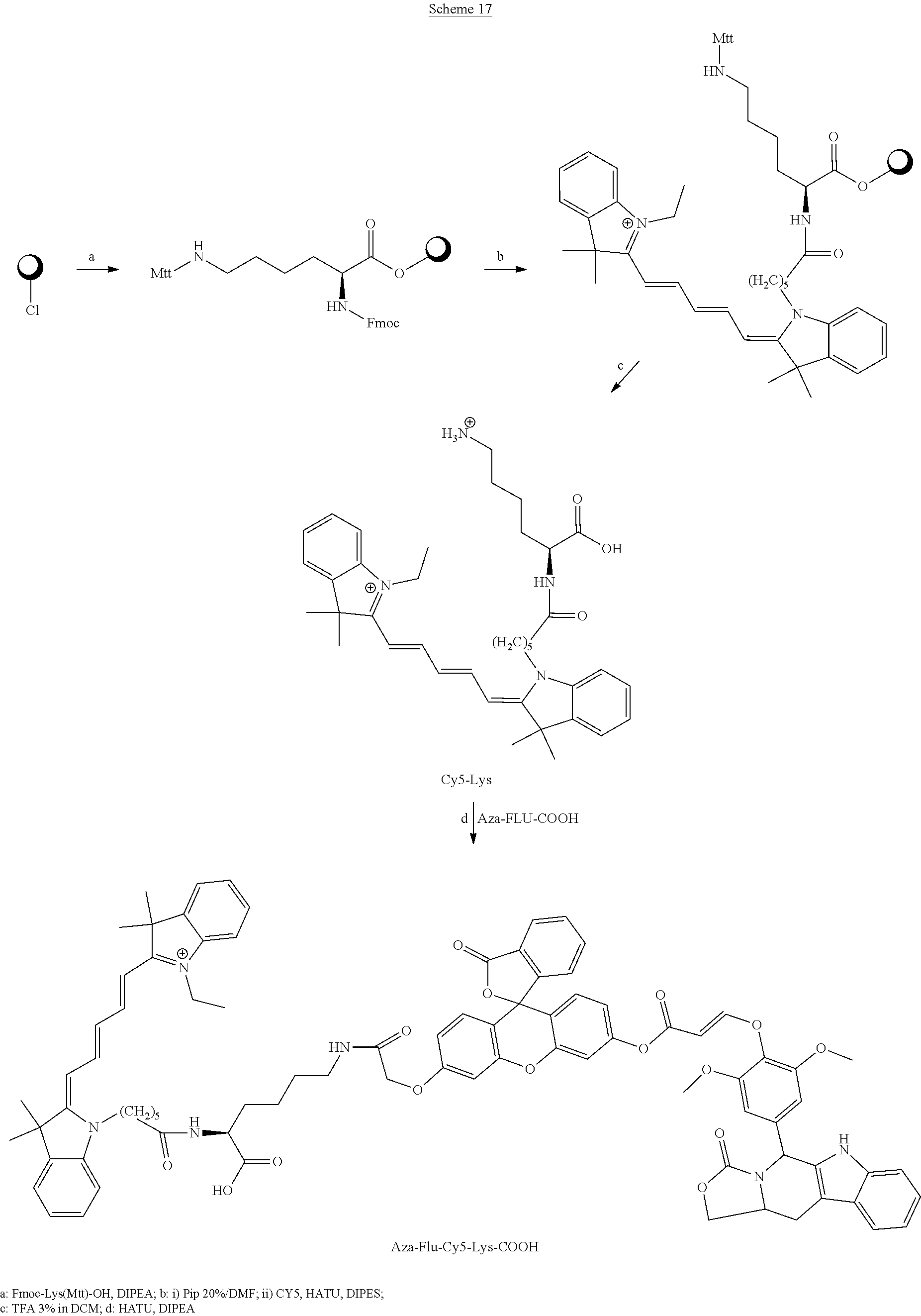

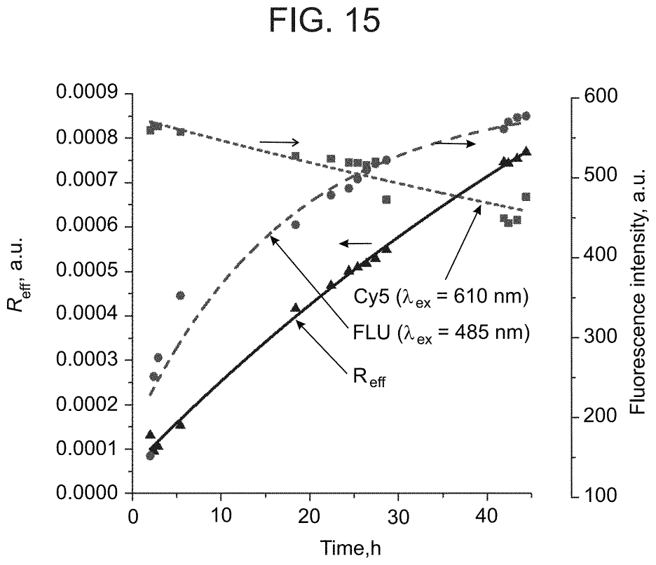

FIG. 15 illustrates an application of the double-fluorophore conjugate Aza-FLU-Cy5-Lys-COOH for the ratiometric monitoring of release of the anticancer drug azatoxin, wherein R_{eff} was calculated using the ratio between I_{FLU} and I_{Cy5}.

### Example 7

### In Vivo Fluorescence Imaging

Monitoring and quantification of drug release in mouse models (male athymic nude mice), is effected as described in the art [Yuan L., Lin W., Zhao S., Gao W., Chen B., He L., Zhu S. A unique approach to development of near-infrared fluorescent sensors for in vivo imaging. J. Am. Chem. Soc., 2012, 134, 13510-13523; and Liu T., Luo S., Wang Y., Tan X., Qi Q., Shi C. Synthesis and characterization of a glycine-modified heptamethine indocyanine dye for in vivo cancer-targeted near-infrared imaging. Drug Des. Dev. Ther. 2014, 8, 1287-1297]. For quantification and drug-release profiling, one can follow the procedures described in the literature [Bazylevich A., Patsenker L. D., Gellerman G. Exploiting fluorescein based drug conjugates for fluorescent monitoring in drug delivery. Dyes and Pigments, 2017, 139, 460-472; and Shkand T. V., Chizh M. O., Sleta I. V., Sandomirsky B. P., Tatarets A. L., Patsenker L. D. Assessment of alginate hydrogel degradation in biological tissue using viscosity-sensitive fluorescent dyes. Methods Appl. Fluoresc., 2016, 4, 044002], using predefined calibration curves. To monitor the time-dependent distribution of the theranostic conjugates, one can use the Maestro™ in vivo fluorescence imaging system. Images are captured using two filter sets to discriminate the signals originating from the reference reporter and those from the switchable dye, and one can use the ratio between these signals to quantify R_{eff}.

### Example 8

### Synthesis and Use of Hydrophilic Dual-Dye Theranostic Conjugate Aza-FLU-Cy5s-Lys-COOH

Scheme 18 below presents a non-limiting example of a hydrophilic dual-fluorophore conjugate, according to some embodiments of the present invention, designed to deliver azatoxin (Aza), and referred to herein as Aza-FLU-Cy5s-Lys-COOH. As can be seen in Scheme 15, the conjugate comprises, circles from left to right, encircle a reference signal moiety (FLU), a spacer (Lys) for attachment of a targeting moiety; a hydrophilic switchable signal moiety (Cy5), a cleavable linker (FLU); and a bioactive agent (Aza).

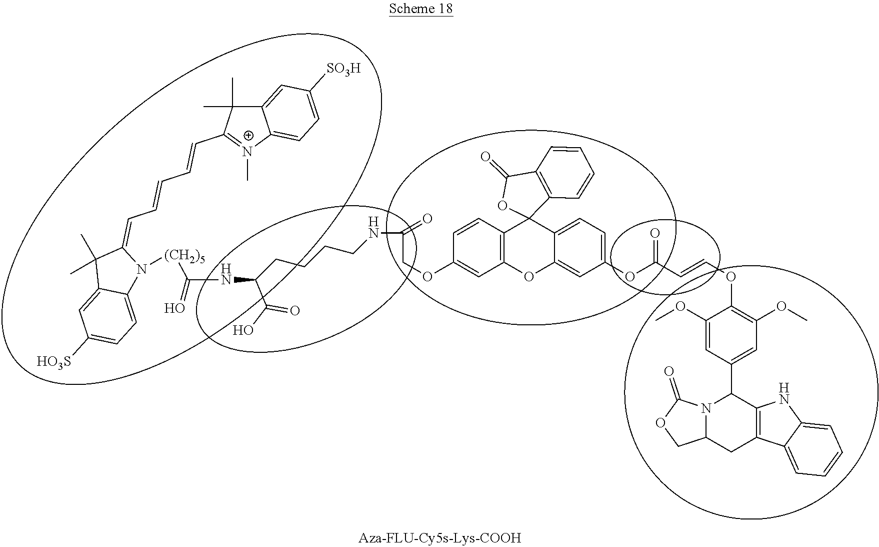

### Synthesis of Aza-FLU-Cy5s-Lys-COOH:

Aza-Flu-COOH (64.5 mg, 0.078 mmol), HATU (29.6 mg, 0.078 mmol) and DIPEA (67.8 μL, 0.39 mmol) were dissolved in DMF (1 mL) and vortexed for 1 minute. The obtained solution was added to each vial that contained 57.7 mg (0.078 mmol) of Cy5s-Lys and reaction mixtures were shaken 1 hour in the dark. After completion of the reaction and evaporation of the solvent, the residues were purified on preparative HPLC and lyophilized to give the final Aza-Flu-Cy5s (4.1 mg, 10% yield) as violet powders, respectively. HPLC: Rf=8.473 min (254 nm, gradient 5-100% AcCN-H2O+0.1% FA). HRMS: Calc.: (M)+1575.5047, Found: (M)+=1575.5093, (M+H)2+=787.7587.

### Example 9

### Fluorescent Intensity Based and Ratiometric Monitoring of Drug Delivery and Release by Using Aza-FLU-Cy5-Lys-COOH and Aza-FLU-Cy5s-Lys-COOH Conjugates

Scheme 19 below presents a chemical reaction used for fluorescent monitoring of drug delivery and drug release by using Aza-FLU-Cy5-Lys-COOH and Aza-FLU-Cy5s-Lys-COOH conjugates.

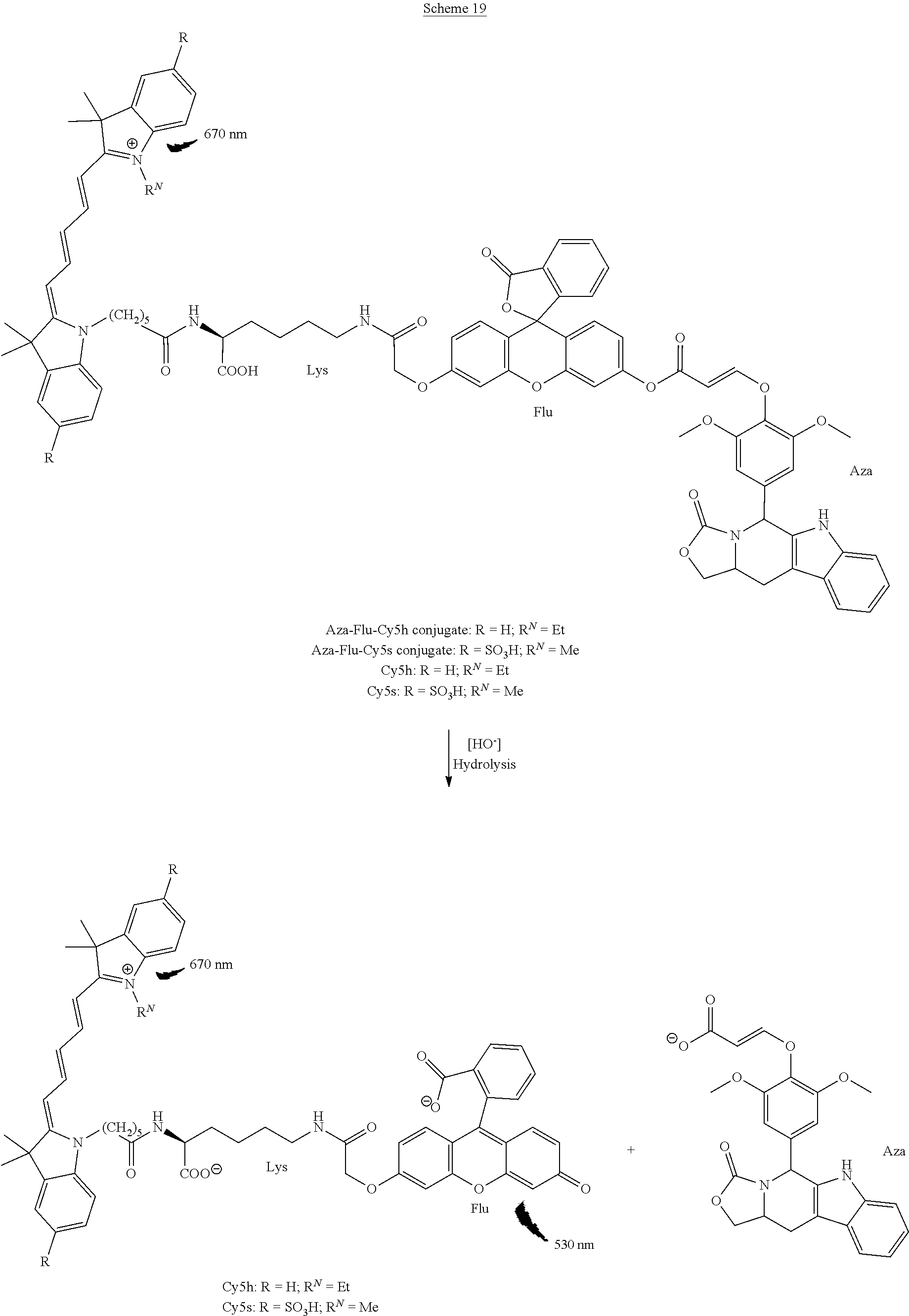

As can be seen in Scheme 19, the hydrolytic cleavage of acrylate ester linker in the red-fluorescent Aza-FLU-Cy5-Lys-COOH and Aza-FLU-Cy5s-Lys-COOH conjugates to release Aza and form green-red dual fluorescent conjugates Flu-Cy5 and Flu-Cy5s.

The time-dependent fluorescence excitation and emission spectra of Aza-Flu-Cy5h and Aza-Flu-Cy5s in PB, CM, and in the presence of 1.25% LH were measured (spectra not shown). To detect the Flu signal in the “On” form, the fluorescence excitation spectra were taken at the registration wavelength λ_{Reg}=560 nm while the Cy5 signal was obtained at λ_{Reg}=720 nm. The fluorescence spectra of Aza-Flu-Cy5h and Aza-Flu-Cy5s were excited at λ*=440 nm, 480 nm, and 610 nm. The λ*=610 nm enabled exclusive excitation of Cy5 while λ*=440 nm and 480 nm provided predominant excitation of Flu. Based on these time-dependent spectra, the drug cleavage profiles were generated and the reaction half-lives (τ_{1/2}) were obtained by using first-order exponential decay functions.

The initial fluorescence excitation spectra of Aza-Flu-Cy5h and Aza-Flu-Cy5s (Time=0) measured at λ_{Reg}=720 nm and fluorescence spectra measured at λ*=610 nm exhibit a strong absorption (λₘₐₓAb ˜645 nm) and fluorescence (λₘₐₓFl ˜665 nm) of Cy5. At the same time, only a very weak Flu absorption (λₘₐₓAb ˜460 nm) and emission (λₘₐₓFl ˜519 nm) are observed at Time=0 in the fluorescence excitation spectra of Aza-Flu-Cy5h and Aza-Flu-Cy5s measured at λ_{Reg}=560 nm and in the fluorescence spectra measured at λ*=440 nm and 480 nm. The absorption and emission bands of Flu (λₘₐₓFl ˜453 nm and 519 nm) overlap with the CM and LH autoabsorption and autofluorescence bands (λₘₐₓFl ˜450 nm and 536 nm, respectively).

When incubated in PB at 37° C., Aza-Flu-Cy5h and Aza-Flu-Cy5s demonstrate a very slow drug release: the emission spectra remain almost unchanged for at least 48 hours. A more pronounced and much faster change was observed, when Aza-Flu-Cy5h and Aza-Flu-Cy5s were incubated in the presence of 1.25% LH. The excitation spectrum of Aza-Flu-Cy5h measured at λ_{Reg}=560 nm exhibits a noticeable, 6-fold increase in the Flu band (λₘₐₓ ˜454 nm) upon 48 hours incubation. The fluorescence spectra excited at 440 nm and 480 nm comprise two bands: the Flu band (λₘₐₓFl ˜519 nm) that also exhibits a 6-fold increase over time and a weak Cy5h band (λₘₐₓFl ˜665 nm), which simultaneously increases due to the FRET from Flu. The half-life of the drug release rate estimated from the excitation and fluorescence spectra is about τ_{1/2} ˜5.8-5.9 hours. When excited at 610 nm, the Cy5h intensity remains almost unchanged. The Cy5h signal obtained with λ*=440 nm and 480 nm cannot be utilized for the ratiometric measurements of drug release because it is dependent on the Flu signal. At the same time, the signal obtained with λ*=610 nm is useful for ratiometry.

The drug release rate for the Aza-Flu-Cy5s conjugate is about 2.7 times faster (τ_{1/2} ˜15.3-16.6 h) compared to Aza-Flu-Cy5h, which could be due to better accessibility of the acrylate ester bond for esterases. The intensities of the Flu bands increase by a factor of about 10.

Remarkably, the drug release for the Aza-Flu-Cy5h and Aza-Flu-Cy5s conjugates is slow compared to that for Aza-Flu-COOH (˜1.8 h). This may be due to the steric hindrance caused by Cy5 moiety to the acrylate ester bond when interacting with esterases.

The Cy5h fluorescence excitation and emission peaks in the Aza-Flu-Cy5h conjugate remain almost unchanged within 24 h and then begin to slowly decrease. In contrast, Cy5s dye in the Aza-Flu-Cy5s conjugate was found to be less stable. During 48 h, it shows about 3.5-fold steady decline in the excitation and emission bands. This decrease is more likely due to the degradation of the conjugated Cy5 dyes in LH. Because the activity of LH reduces over time, the decrease is observed to a certain emission level (about 40% of the initial value for Cy5h and ˜12% for Cy5s) and then the intensities remain almost constant. This stabilization is achieved in about 96 h. Although the Cy5s signal in the Aza-Flu-Cy5s conjugate reduces over time, this decrease is not dependent on the Flu emission and can be utilized for the ratiometric measurements. Importantly, no signs of the Flu degradation in the double-dye systems were observed during at least 600 h.

Surprisingly, the drug cleavage for both conjugates incubated at 37° C. in CM was found to be much slower compared to LH (τ_{1/2} ˜60 h).

Based on the obtained fluorescence excitation and emission profiles, the ratiometric curves expressing the ratio between the Flu and Cy5 signals (F_{Flu}/F_{Cy5}) over time were generated. Both the intensity based and ratiometric curves can be correlated with the concentration of the released drug molecules and the drug release degree by known methods. As compared to the intensity based profiles, the ratiometric curves are known to not depend on the initial Aza-Flu-Cy5h and Aza-Flu-Cy5s concentration and instrumental setup, and therefore, are beneficial for sensing applications.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A conjugate, comprising:
a bioactive agent moiety,
at least two fluorophore moieties, and
a cleavable linker connecting said bioactive agent moiety and said at least one fluorophore moiety, wherein:
said at least one fluorophore moiety is characterized by at least one reference luminescence signal and at least one switchable luminescence signal, and a change in said switchable luminescence signal upon cleavage of said cleavable linker is different than a change in said reference luminescence signal, and
at least one of said at least two fluorophore moieties is characterized by exhibiting said reference luminescence signal and constitutes a reference fluorophore moiety, and at least one other of said at least two fluorophore moieties is characterized by exhibiting said switchable luminescence signal and constitutes a switchable fluorophore moiety,
the conjugate is structured and designed so as to allow monitoring and calibrated luminescence determination of a value related to a release of said bioactive agent from the conjugate.

2-3. (canceled)

4. The conjugate of claim 1, wherein each of said reference luminescence signal and said switchable luminescence signal is independently detectable within a range from 600 nm to 900 nm.

5. The conjugate of claim 1, wherein each of said reference luminescent signal and said switchable luminescence signal comprises at least one distinguishable luminescence intensity of at least one wavelengths, and/or at least one distinguishable luminescence lifetime, and/or at least one distinguishable polarization/anisotropy, and any combination, ratio, product and/or correlation thereof.

6. The conjugate of claim 5, wherein said change in said switchable luminescence signal is at least 10% greater than said change in said reference luminescence signal.

7. The conjugate of claim 1, structured and designed so as to allow theranostic bioavailability at physiological conditions.

8. The conjugate of claim 1, further comprising a targeting moiety.

9. (canceled)

10. The conjugate of claim 1, wherein said switchable fluorophore moiety is selected from the group consisting of: 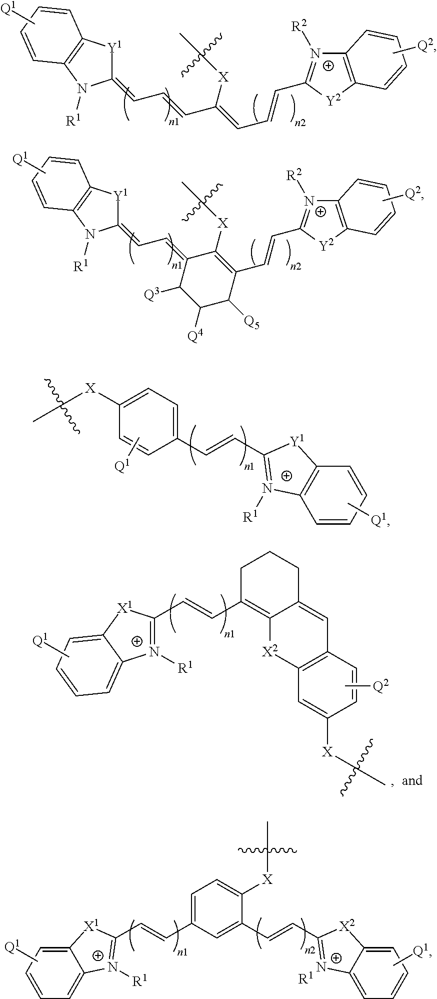
wherein:
X═O, S, Se, NR^{N}, 2-phenyoxy, 4-phenyoxy, aryloxy;
R^{N}=hydrogen, alkyl, aryl alkylaryl, or contain a reactive group or a solubilizing group selected from sulfate, sulphonate, quaternary amine, phosphate, phosphonate and PEG;
Y¹, Y² are independently selected from C(R^{a}, R^{b}), O, S, NR^{N};
R^{a}, R^{b} are independently selected from hydrogen, alkyl, aryl alkylaryl, or contain a reactive group or a solubilizing group selected from sulfate, sulphonate, quaternary amine, phosphate, phosphonate, PEG;
R^{a} and R^{b} can form a ring;
R¹, R² are each independently selected from hydrogen, alkyl, aryl alkylaryl, or contain a reactive group or a solubilizing group selected from sulfate, sulphonate, quaternary amine, phosphate, phosphonate, PEG;
Q¹, Q² are at least one of groups consisting of R¹, halogen, cyano, sulfo, phosphate, carboxy, formyl, alkyl, aryl, alkylaryl, alkoxy, aryloxy or a substituted or unsubstituted cyclic moiety; two adjacent Q¹ and two adjacent Q² can form a substituted or unsubstituted cyclic moiety;
each of 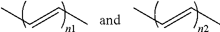
is independently a linear or cyclic, substituted or unsubstituted polyene, and each of n1 and n2 is independently an integer ranging 1-4;
the wiggled line represents attachment to said cleavable linker.

11. The conjugate of claim 1, wherein said reference fluorophore moiety comprises a fluorescent dye selected from the group consisting of a cyanine-based fluorescent dye, a styryl-based fluorescent dye, a squaraine-based fluorescent dye, a squaraine-rotaxane-based fluorescent dye, a phthalocyanine-based fluorescent dye, a porphyrine-based fluorescent dye, a xanthene-based dye, a phenothiazine-based dye, a luminescent metal-ligand complex, a fluorescent protein, a luminescent nanoparticle, a luminescent quantum dot, a luminescent nanocrystal, a luminescent polymeric particle, a tandem fluorophore, or a fluorescent dye selected from Cy, Dy, Alexa Fluor, IRDye, LiCor, BODIPY, SETA dye series.

12. The conjugate of claim 8, wherein said targeting moiety is selected from the group consisting of a peptide, a protein, an antibody and a nanoparticle.

13. The conjugate of claim 12, wherein said targeting moiety is selected from the group consisting of octreotide (OCT), lanreotide, pasireotide, vapreotide, cilengitide analog c(RGDfK), and luteinizing Hormone-Releasing Hormone (LHRH), bombesin, and arginine-glycine-aspartic acid (RGD).

14. The conjugate of claim 12, further comprising a spacer moiety linking said targeting moiety and said at least one fluorophore moiety.

15. The conjugate of claim 1, wherein said cleavable linker comprises an ester, an amide, a carbamate, a carbonate, a disulfide, a sulfonamide, an ether, a thioether, a valine-citrulline, a hydrazine and an oxyacrylate.

16. The conjugate of claim 1, wherein said bioactive agent is selected from the group consisting of a drug, a photodynamic therapy sensitizer, radiotherapy agent, a metal complex, an anti-cancer agent, an anti-proliferative agents, chemosensitizing agents, an anti-inflammatory agent, an antimicrobial agent, an anti-oxidant, a hormone, an anti-hypertensive agent, an anti-diabetic agent, an immunosuppressant, an enzyme inhibitor, a neurotoxin and an opioid.

17. The conjugate of claim 16, wherein said bioactive agent is a drug selected from the group consisting of chlorambucil, azatoxin, an antimitotic, dolastatin 10, monomethyl auristatin F, monomethyl auristatin E, maytansine (DM1), a Topo I irinotecan inhibitor, 7-ethyl-10-hydroxy-camptothecin (SN-38), a DNA minor groove binding alkylating agent, duocarmycin, adozelesin, bizelesin and carzelesin.

18. The conjugate of claim 16, wherein said sensitizer is photo-activated upon cleavage of said cleavable linker.

19. The conjugate of claim 16, wherein said sensitizer comprises a dye selected from the group consisting of a cyanine-based dye, a styryl-based dye, a squaraine-based dye, a phthalocyanine-based dye, and a porphyrine-based dye, xanthene-based dye, a phenothiazine-based dye, a iodinated dye, a brominated dye, a chlorin-based dye, a bacteriochlorin-based dye, a fullerene-based dye, a metal-ligand complex, a halogenated dye, a nanoparticle, a photofrin-based dye, a photoporphyrin-based dye, a benzoporphyrin-based dye, a tookad-based dye, an antrin-based dye, a purlytin-based dye, a foscan-based dye, a iodinated, brominated or a mixed iodinated cyanine-based or squaraine based dye, and any combination thereof.

20. A method of calibrated luminescence determination of a value related to a release of a bioactive agent in a tissue, comprising:
scanning the tissue with a probe designed to detect and record said reference luminescence signal and said switchable luminescence signal;
contacting the tissue with the conjugate of claim 1;
monitoring a change in said reference luminescence signal and said switchable luminescence signal for a predetermined period of time;
calculating the value related to a release of the bioactive agent according to the following equation:
R_{eff}˜I_{Swi signal}/I_{Ref signal}, or
R_{eff}=k(I_{Swi signal}/I_{Ref signal})
wherein:
I_{Swi signal} is a value representing said switchable luminescence signal,
I_{Ref signal} is a value representing said reference luminescence signal,
and
k is an experimentally determined calibration coefficient.
